(19) United States
(12) Patent Application Publication
(10) Pub. No.: US 2011/0251158 A1
(43) Pub. Date: Oct. 13, 2011

(54) FULLERENE THERAPIES FOR INFLAMMATION AND INHIBITION OF BUILD-UP OF ARTERIAL PLAQUE

(75) Inventors: Christopher L. KEPLEY, Ringgold, VA (US); Robert P. LENK, Danville, VA (US); Darren K. MACFARLAND, Danville, VA (US); Zhiguo ZHOU, Winston-Salem, NC (US)

(73) Assignee: Luna Innovations Incorporated, Roanoke, VA (US)

(21) Appl. No.: 12/884,076

(22) Filed: Sep. 16, 2010

Related U.S. Application Data
(63) Continuation-in-part of application No. 12/921,049, filed on Sep. 3, 2010.
Application No. 12/921,049 is a continuation-in-part of application No. 12/921,083, filed on Dec. 16, 2010.
(60) Provisional application No. 61/033,309, filed on Mar. 3, 2008.
(60) Provisional application No. 61/033,336, filed on Mar. 3, 2008.

(51) Int. Cl. A61K 31/685; C07C 69/76; A61K 31/4192; C07D 249/04; A61P 19/02; C07D 401/14; C07F 9/10; A61K 31/66; A61P 9/00; A61K 31/235; A61K 31/455

(52) U.S. Cl. 514/78; 514/510; 560/80; 514/359; 548/255; 514/332; 546/263; 554/80; 514/107

(57) ABSTRACT

Described herein are methods for treating inflammatory disorders or for inhibiting the build-up of arterial plaque. The methods comprise administering to a subject in need thereof a therapeutically effective amount of a synthetically modified fullerene.

### CROSS-REFERENCE TO RELATED APPLICATIONS

This application is a continuation-in-part of U.S. application Ser. No. 12/921,049, filed on Sep. 3, 2010, which in turn is a national stage of PCT/US2009/001333 filed on Mar. 3, 2009, published as WO 2009/114088, which in turn claims priority of U.S. Provisional Application No. 61/033,309, filed on Mar. 3, 2008; and of U.S. application Ser. No. 12/921,083, filed on Sep. 3, 2010, which in turn is a national stage of PCT/US2009/001335 filed on Mar. 3, 2009, published as WO 2009/114090, which claims priority of U.S. Provisional Application No. 61/033,336, filed on Mar. 3, 2008, the entire content of each of which is incorporated herein by reference.

### BACKGROUND

Various embodiments described herein relate to the use of fullerenes to treat inflammatory disorders and to inhibit the build-up of arterial plaque in human.

Inflammation is the complex biological response of tissues to harmful stimuli, such as pathogens, damaged cells, or irritants. However, inflammation which runs unchecked can lead to a host of disorders, such as inflammatory arthritis, rheumatoid arthritis, allergic disease (hay fever), and atherosclerosis.

Mast cells (MC) are granule-rich tissue cells that significantly contribute to a wide range of diseases through the release of noxious mediators. Peripheral blood basophils (PBB) are similar to MC in that they are the only leukocytes that contain pre-stored histamine within their granules. The high affinity IgE receptor, FcεRI, is one of many ways in which MC/PBB can be activated for mediator release. Cross linking IgE-primed FcεRI leads to the release of various pre-formed and newly generated mediators which can cause allergic disease. Ryan, J. J., Kashyap, M., Bailey, D., Kennedy, S., Speiran, K., Brenzovich, J., Barnstein, B., Oskeritzian, C., and Gomez, G. Crit Rev. Immunol. 27, 15-32 (2007). Moreover, MC are suspected to play a role in other inflammatory disorders such as arthritis and cardiovascular disease through non-FcεRI mediated mechanisms. Kovanen, P. T. Immunol. Rev. 217, 105-122 (2007). Basophils, also established effector cells in allergic disease, have recently been implicated as playing a major role in adaptive and innate immunity. Alter, S. C. and Schwartz, L. B. Biochim. Biophys. Acta 991, 426-430 (1989).

Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue. Acute inflammation is a short-term process which is characterized by the classic signs of inflammation—swelling, redness, pain, heat, and loss of function—due to the infiltration of the tissues by plasma and leukocytes. It occurs as long as the injurious stimulus is present and ceases once the stimulus has been removed, broken down, or walled off by scarring (fibrosis).

Prolonged inflammation, known as chronic inflammation, leads to a progressive shift in the type of cells which are present at the site of inflammation and is characterized by simultaneous destruction and healing of the tissue from the inflammatory process. Chronic inflammation is a pathological condition characterized by concurrent active inflammation, tissue destruction, and attempts at repair. Chronic inflammation is not characterized by the classic signs of acute inflammation listed above. Instead, chronically inflamed tissue is characterized by the infiltration of mononuclear immune cells (monocytes, macrophages, lymphocytes, and plasma cells), tissue destruction, and attempts at healing, which include angiogenesis and fibrosis. Tissue mast cells contain many of the mediators that are released that mediate this influx of inflammatory cells. Endogenous causes include persistent acute inflammation. Exogenous causes are varied and include bacterial infection, prolonged exposure to chemical agents such as silica, or autoimmune reactions such as rheumatoid arthritis.

Cells of the immune system use a signal cascade to mount an escalating response to a real or perceived insult. The inflammatory response becomes pathogenic when the signal cascade is invoked inappropriately. For example, autoimmune diseases are the consequence of the immune system mounting a response against antigens which are intrinsic. Many anti-inflammatory agents function by inhibiting the signal cascade, such as by blocking intracellular or intercellular effectors. Glucocorticoids, for example, mimic the natural immune suppressant, cortisol, to block genes at the transcription level, and cylco-oxygenase inhibitors are small molecules that bind to and inhibit enzymes that process an internal signal molecule in cells.

Cardiovascular disease is a major health risk throughout the industrialized world. Atherosclerosis, the most prevalent of cardiovascular diseases, is the principal cause of heart attack, stroke, and gangrene of the extremities, and thereby the principal cause of death in the United States. Atherosclerosis is a complex disease involving many cell types and molecular factors (for a detailed review, see Ross, Nature 362:801-809 (1993)).

Atherosclerosis is a disease characterized by the deposition of fatty substances, primarily cholesterol, and subsequent fibrosis in the inner layer (intima) of an artery, resulting in plaque deposition on the inner surface of the arterial wall and degenerative changes within it. The ubiquitous arterial fatty plaque is the earliest lesion of atherosclerosis and is a grossly flat, lipid-rich atheroma consisting of macrophages (white blood cells) and smooth muscle fibers. The fibrous plaque of the various forms of advanced atherosclerosis has increased intimal smooth muscle cells surrounded by a connective tissue matrix and variable amounts of intracellular and extracellular lipid. At the luminal surface of the artery, a dense fibrous cap of smooth muscle or connective tissue usually covers this plaque or lesion. Beneath the fibrous cap, the lesions are highly cellular consisting of macrophages, other leukocytes and smooth muscle cells. Deep in this cell-rich region may be areas of cholesterol crystals, necrotic debris and calcification.

If allowed to progress, the disease can cause narrowing and obstruction of the lumen of the artery, diminished or occluded blood flow and, consequently, ischemia or infarction of the predominantly affected organ or anatomical part such as the brain, heart, intestine or extremities. The result can be significant loss of function, loss of cellular substance, emergency medical and/or surgical procedures, and significant disability or death. Alternatively, the arterial wall can be severely weakened by the infiltration of the muscular layer with the lipid (cholesterol), inflammatory white blood cells, connective tissue and calcium, resulting in soft and/or brittle areas which can become segmentally dilated (aneurysmal) and rupture or crack leading to organ, limb or even life-threatening hemorrhage.

Once the disease has progressed to the stage of significant persistent symptoms and compromised function, the next treatment step has conventionally been artery bypass grafting to repair and/or replace the damaged artery. While coronary artery bypass has become one of the more common major cardiovascular surgical procedures in the United States, surgery clearly is not the solution to the pathologic process. Moreover, there is a significant risk of morbidity and mortality associated with surgery that many patients are reluctant to accept. Indeed, the autogenous veins or arteries used to bypass the disease-impaired arteries undergo atherosclerosis changes postoperatively generally at a faster rate than the original, affected arteries. The Coronary-Artery Surgery Study (CASS) sponsored by the National Heart, Lung and Blood Institute (NHLBI) concluded that certain subsets of patients do not gain any overall statistical benefit from bypass surgery in comparison to other medical treatments. Carraciolo, Circulation, 91(9): 2335-44 (1995).

As an alternative to coronary bypass surgery, certain medications and procedures are used to treat the results of atherosclerosis. These treatments include chelation with ethylene diamine tetra-acetic acid (EDTA) and percutaneous transluminal coronary angioplasty (PTCA). EDTA treatments, however, are still experimental, unproved and potentially as harmful as they are beneficial. PTCA treatments are invasive, of limited application and success and occasionally manifest lethal complications. Highly experimental intra-arterial laser beam plaque vaporization has limited application and requires an open operative approach to affected vessels.

It is now well established that vascular blockage and cardiovascular disorders including myocardial infarction, coronary heart disease, hypertension and hypotension, cerebrovascular disorders including stroke, cerebral thrombosis and memory loss due to stroke; peripheral vascular disease and intestinal infarction are caused by blockage of arteries and arterioles by atherosclerotic plaque. The production of atherosclerotic plaque formation is multi-factorial in its production. Hypercholesterolemia, especially elevated levels of low-density lipoprotein cholesterol (LDL) is an important risk factor for atherosclerosis and arteriosclerosis and associated diseases.

Lipoproteins are spherical particles with the lipophilic triglycerides and cholesteryl esters in the hydrophobic core, and the amphiphilic lipids, phospholipids and free cholesterol on the surface with apolipoproteins. When the amount of cholesterol entering the body increases, the pools of sterol within liver cells expands and the receptors that clear LDL from the blood down-regulate, thus increasing LDL levels in the blood. When cholesterol intake is constant, some long-chain saturated fatty acids further suppress the hepatic LDL receptor whereas several unsaturated fatty acids have the opposite effect. Lipoprotein (a) [Lp (a)] has emerged as a plasma lipoprotein linked to both diseases of the coronary arteries, the carotid and the cerebral arteries. It is structurally related to LDL and possesses one molecule of apolipoprotein B₁₀₀ per particle. Macrophages express the scavenger receptor that readily recognizes oxidatively modified Lp (a). Marcovina & Morrisett, Current Opinion In Lipidology, 6:136-145 (1995).

Cholesterol levels below 200 mg/dl are considered “desirable.” A Scandinavian study showed that reduction of cholesterol reduced mortality associated with coronary artery disease (CAD) by 42% over six year period and reduced overall mortality by 30%. J. Hardman & L. Lipman, Goodman & Gilman's The Pharmacological Basis Of Therapeutics (9th ed. 1996) (hereinafter “J. Hardman”). Researchers have shown that a 1-mmol/L increase in triglyceride levels produces a 76% increase in cardiovascular disease risk in women and a 31% increase in men. Austin, American Journal of Cardiology, 83 (9B):13F-16F (1999). Even in patients with established disease, lowering of LDL cholesterol to between 2 and 2.5 mmol/L retards its progression and may even lead to regression. Illingsworth, Drugs, 41(20):151-160 (1991).

It is recommended that persons with elevated cholesterol concentrations above 240 mg/dL (6.2 mM/L) receive treatment and that those with borderline values between 200-239 mg/dL (5.2 to 6.2 mM/L) be further evaluated according to the presence of risk factors for coronary artery disease including the sex of the patient, post-menopausal status, a low plasma concentration of high-density lipoprotien cholesterol (HDL) cholesterol (below 35 mg/dL [0.9 mM/L]), positive family history, smoking, hypertension and diabetes mellitus. Expert Panel on Detection, Evaluation, and Treatment of High Blood Cholesterol in Adults, J. Am. Medical A., 269(23):3015-3023 (1993). Other factors include obesity, hypertriglyceridemia, sedentary lifestyle, steroid use, .beta.-adrenergic blocking agents, some diuretics and genetic factors. Frohlich & Pritchard, Clinical Biochemistry, 22:417-433 (1989).

By the 1980's, it was recognized that HDL levels could be more important in predicting atherosclerotic disease than LDL and that HDL may prevent the development of CAD. Id. Factors such as smoking, obesity, hypertriglyceridemia, genetic factors and lack of exercise are major causes of reduced serum HDL. HDL cholesterol lipoproteins move excess cholesterol from the extrahepatic organs to the liver for excretion. Dietschy, Am. J. Clinical Nutrition, 65:1581 S-9S (1997). There is evidence that virtually every body tissue is capable of at least some cholesterol synthesis from the precursor acetyl-coenzyme A (CoA). Every day, HDL carries back to the liver an amount of cholesterol equal to the amount synthesized and taken up as LDL by all extrahepatic organs except endocrine glands. There is a second LDL transport process that is receptor independent. Id. Removal of free cholesterol from arterial wall cells may be an important mechanism by which HDL plays an anti-atherogenic role. J. Hardman, supra, at 878.

The earliest recognized gross lesion in atherogenesis is the fatty streak, characterized by an accumulation of cells loaded with cholesteryl esters (“foam cells”) just beneath the vascular endothelium. The LDL receptor in the arteries gives rise to foam cells and fatty streaks, the earliest lesion in atherosclerosis, but there is also a receptor-independent mechanism for their formation. This has been demonstrated by the development of lesions rich in macrophage-derived foam cells, even in patients and animals deficient in LDL receptors, and the failure to produce foam cells from normal monocytes and monocyte derived macrophages incubated with LDL. This led researchers to explore the possibility of a post-secretory modification of LDL before it is taken up into foam cells by a new, specific receptor: the “scavenger receptor.” Steinberg, New Eng J. Medicine, 320(14): 915-924 (1989).

At any given level of hypercholesterolemia there is considerable variation in clinical disease. Postsecretory modifications in the structure of lipoproteins appear to affect their atherogenic potential. Steinberg, supra, at 915. It is not only the elevated levels of LDL cholesterol that are important, but also its oxidation that leads to atherosclerosis. For this reason, antioxidants are believed to reduce the risk of atherosclerotic disease. Mortensen, Molecular Aspects of Medicine, 18:s137-s144, (Supp. 1997). Peroxidation of polyunsaturated fatty acids in the LDL lipids is the common initiating factor of the changes and the cytotoxicity of oxidized LDL has been proven by several research groups and may lead to the denudation of the benign fatty-streak lesion into the atheromatous plaque. Steinberg, supra, at 918.

Researchers believe that the oxidation of LDL within the arterial wall itself is most important. Ocana, New Eng. J. Medicine, 321(17):1196-1197 (1989). Auto-antibodies to MDL-LDL were seen at significantly higher titers in men with atherosclerosis than in normal controls, and in a greater proportion of smokers, those with higher LDL cholesterol, and those with higher serum levels of copper in the case group. Salonen, 339 LANCET 883-887 (1992).

Researchers also have studied the effects of incubation of LDL with macrophages and found that in that environment LDL is oxidized and recognized and taken up by the acetyl LDL or scavenger receptor in the same cell. Alpha-tocopherol, butylated hydroxytoluene (BHT) and Probucol block this process. Parthasarathy, Arteriosclerosis, 6(5):505-10 (1986). Treatment with Probucol, a potent anti-oxidant, significantly lowered the rate of development of fatty streak lesions in hyperlipidemic rabbits, although the plasma cholesterol level was not lower than in lovastatin-treated animals. Carew, Schwenke & Steinberg, PNAS USA, 84:7725-7729 (1987). Similar results have been demonstrated in cultures of LDL with endothelial cells. Steinbrecher, PNAS, 81:3883-3887 (1984). Monocytes and neutrophils, when incubated with LDL, oxidize LDL and render it toxic. Cathcart, Morel & Chisolm, J. Leukocyte Biology, 38:341-350 (1985).

Fullerene molecules are a family of carbon allotropes that comprise closed cages of generally 60 to 200 carbon atoms and may also include chemical moieties attached to the exterior or incorporated within the cage. Fullerenes can be in the form of a hollow sphere, ellipsoid, or tube. The most common fullerene to date is the C₆₀ Buckminsterfullerene (IUPAC name (C₆₀-Ih)[5,6]fullerene). Another fairly common buckminsterfullerene is C₇₀, but fullerenes with 72, 76, 84 and even up to 100 carbon atoms are commonly obtained. Fullerene molecules can contain as few as 20 or more than 500 carbon atoms. Fullerenes may enclose one or more atoms such as metal atoms, or other small chemical groups, inside the carbon cage; such fullerenes are sometimes called endohedral fullerenes. Fullerenes may also be modified or derivatized to include chemical functional groups attached to the surface of the carbon cage.

Structural variations include nonclosed-cage structures, heterofullerenes, derivatives formed by substitution of hydrofullerenes, the fusion of organic rings or ring systems to the fullerene cage, chiral fullerenes, buckyball clusters, nanotubes, megatubes, polymers, nano “onions,” linked “ball-and-chain” dimers, and fullerene rings. See, e.g., Miessler and Tarr, Inorg. Chem. 3, Pearson Education International. ISBN 0-13-120198-0 (2004); Mitchel et. al., Inorg. Chem., 40: 2751 (2001); Sano, Nature (London), 414: 506 (2001); Shvartsburg, Phys. Chem. 103: 5275 (1999); and Li et al., Chem. Phys. Lett. 335: 524 (2001).

In general, fullerenes are hydrophobic and sparingly soluble in many solvents. See, e.g., Braun et al., Fullerenes, Nanotubes and Carbon Nanostructures, 15; 311-314 (2007). However, a variety of procedures for functionalizing fullerenes are known in the art, and some of the derivative fullerenes are water soluble. See, e.g., U.S. Pat. No. 5,648,243 to Chiang; U.S. Patent Application Publication Nos. 2008/0004345 and 2004/0044062; Jensen et al., Bioorganic & Medicinal Chemistry, 4:767-79 (1996); Da Ros et al., Croatica Chemica Acta CCACAA 74:743-55 (2001); Wilson, Perspectives in Fullerene Nanotechnology, Osawa, ed., (Kluwer Academic Publishers, Dorcrecht, Netherlands, 2000); Syrensky, et al., Kopf Carrier #63, (David Kopf Instruments Tujunga, California, September 2006); Y. L. Lai and L. Y. Chiang, J. Autonomic Pharmacol., 17:229 (1997); Schinazi et al., Proc. Electrochem. Soc., 97:10, (1997); Lai et al., World J. Surg., 24:450 (2000); Jin et al., J. Neuroscience Res., 62:600 (2000); Huang et al., Free Radical Biol. Med., 30:643 (2001); Chi et al., Perspectives of Fullerene Nanotechnology, pp 165-183, E. Osawa ed., (Kluwer Academic Publisher, Great Britain, 2002); Dugan et al., P.N.A.S. 94:9434-39 (1997); Dugan et al., Parkinsonism & Related Disorders 7:243-46 (2001); Quick et al., Neurobiol of Aging (electronic publication 2006); Kato et al., Chem & Biodiv., 2:1232-1241 (2005); Georgakilas et al, Proc. Nat. Acad. Sci. 99:5075-5080 (2002).

Incorporation of fullerenes into lipid vesicles has also been studied (see, e.g., Bensasson et al., Journal of Physical Chemistry, 98:3492-3500 (1994); Hirsch et al., Angewandte Chemie International Edition, 39:1845-1848 (1999); U.S. Pat. No. 7,070,810; Felder, et al., Helv. Chim. Acta, 85:288-319 (2002).

Fullerenes can also be modified at their surface to present specific biologically active groups, such as lectins or antibodies. See, e.g., U.S. Patent Application Publication No. 2005/0043787; U.S. Pat. No. 5,310,669. Certain chemically modified fullerenes are commercially available. See, e.g., BuckyUSA, Houston, Tex. and American Dye Source, Inc., Quebec, Canada.

Fullerenes and derivatives of fullerenes have been proposed as free radical scavengers. See, e.g., Haddon, J. Am. Chem. Soc. 112:3389 (1990); U.S. Pat. No. 5,648,243 to Chiang, U.S. Patent Application Publication No. 2003/0162837 by Dugan; U.S. Pat. No. 7,163,956 to Wilson; Kepley, J. Immunol. 179:665 (2007).

### SUMMARY

Described herein are methods for treating inflammatory disorders, comprising administering to a subject in need thereof a therapeutically effective amount of a synthetically modified fullerene.

Also disclosed herein are methods for stabilizing mast cells to prevent mast cell-driven disease such as asthma, arthritis, and allergy, and methods for inhibiting the build-up of arterial plaque in an individual.

According to various embodiments, disclosed herein are methods for treating inflammatory disorders or for inhibiting the build-up of arterial plaque in an individual, comprising administering to the subject in need thereof a therapeutically effective amount of fullerenes.

### BRIEF DESCRIPTION OF THE DRAWINGS

FIGS. 1 to 4 illustrate non-limiting examples of synthetically modified fullerenes.

FIG. 5 illustrates the attenuation of arthritis in fullerene-treated mice. FIG. 5A shows clinical indices, and FIG. 5B shows ankle thickness.

FIG. 6 illustrates the serum levels of TNF-α at day 14 in control and mice treated with fullerene derivatives.

FIG. 7 shows data of affected genes by fullerene derivative preincubation.

FIG. 8 shows inhibition of MC degranulation by fullerene derivatives.

FIG. 9 shows inhibition of MC cytokine production by fullerene derivatives.

FIGS. 10A and 10B show concentration (dose)-dependent inhibition of histamine release and IL-13 release.

FIG. 11 shows inhibition of anti-FcεRI-dependent increases in intracellular calcium and ROS levels by fullerene derivatives.

FIG. 12 shows FcεRI-mediated activation of early signaling molecules is inhibited by fullerene derivatives.

FIG. 13 shows microarray analysis of FcεRI activated genes affected by TGA.

FIG. 14 shows microarray analysis of FcεRI activated genes affected by Inos.

FIG. 15 shows that fullerene derivatives blunt MC-driven anaphylaxis in vivo. FIG. 15A: Average change of temperature with TGA injection; FIG. 15B: Average change of temperature with Inos injection; FIG. 15C: serum histamine production.

FIG. 16 shows effects on lipid uptake by fullerene derivatives.

FIG. 17 shows effects on foam cell formation by fullerene derivatives.

FIG. 18 shows inhibition of clumping of activated monocytes by fullerene derivatives.

FIG. 19 shows inhibition of induction of foam cell formation by fullerene derivatives.

FIG. 20 illustrates an exemplary synthesis scheme for producing Compound 5.

FIG. 21 illustrates an exemplary synthesis scheme for producing C₇₀-tetraglycolic acid, compound 7.

FIG. 22 illustrates an exemplary synthesis scheme for producing C₇₀-tetrainositol, compound 10.

FIG. 23 illustrates an exemplary synthesis scheme for producing C₇₀ TEG acid (TTA), compound 12.

FIG. 24 illustrates an exemplary synthesis scheme for producing C₇₀ with a phenyl propionic acid group as one of its hydrophilic groups.

### DETAILED DESCRIPTION

In accordance with this detailed description, the following definitions apply.

As used herein, the singular forms “a”, “an”, and “the” include plural referents unless the context clearly dictates otherwise. Thus, for example, reference to “compounds” includes a plurality of such compounds and reference to “the dosage” includes reference to one or more dosages and equivalents thereof known to those skilled in the art, and so forth.

### Inflammatory Disorders

The term “inflammatory disorder” or “inflammatory disease” is used to refer to abnormalities associated with inflammation, and comprises a large group of disorders. An inflammatory disorder can be associated with acute inflammation and/or chronic inflammation. Examples of inflammatory disorders include, without limitation, autoimmune diseases, inflammatory arthritis, rheumatoid arthritis, osteoarthritis, gouty arthritis, shoulder tendonitis or bursitis, polymyalgia rheumatica, inflammatory lung disease, asthma, type 1 diabetes melitis, multiple sclerosis, systemic lupus erthematosus, psoriasis, chronic prostatitis, glomerulonephritis, mast cell-mediated type 1 hypersenitivity, hypersensitivity reactions (such as type 2 and type 3 hypersensitivity), inflammatory bowel diseases (such as ulcerative colitis and Crohn's disease), pelvic inflammatory disease, reperfusion injury, transplant rejection, vasculitis, allergic reactions, inflammatory myopathies (such as dermatomyositis, polymyositis, and inclusion body myositis), and leukocyte defects (such as Chediak-Higashi syndrome and chronic granulomatous disease).

Inflammatory arthritis comprises a condition where arthritis is present because of localized joint inflammation. Rheumatoid arthritis, generally considered a type of inflammatory arthritis, involves many joints all of which are damaged to some degree by inflammation and it's sequelae. In certain embodiments, the inflammatory disorder described herein is an inflammatory arthritis, including but not limited to rheumatoid arthritis and mast cells mediated inflammatory arthritis.

Allergic diseases are the result of B cell-produced, specific IgE antibody to common, normally innocuous antigens. In simplistic terms, mast cells and drive the initial, allergen-inducing reaction through the production of IL-4, and other TH2-specific cytokines which result in IgE sensitization. Re-exposure to the allergen triggers an allergic response through the release of inflammatory mediators from mast cells and basophils. The IgE produced binds to FcεRI on mast cells and basophils and the release of pre-allergic mediators is induced when 2 or more IgE molecules are crosslinked with allergen. Mast cells and basophils are unique in driving this process as they are the only cells that express IgE-binding FcεRI receptors that control the release of histamine (prestored only in mast cells and basophils) when the IgE encounters allergen. Indeed, most allergy medications are aimed at neutralizing (anti-histamines, H1-receptor blockers) or preventing (“Omalizumab”) mast cells and basophils FcεRI responses.

Mast cells and basophils also mediate other disease processes. Mice genetically engineered to not express mast cells fail to develop asthma-like pulmonary disease when sensitized with less-aggressive immunization protocols and challenged with aerosolized allergen.

Mast cells also are involved in other disease processes. Mast cells have traditionally been established participants in allergic disease and in protection against extracellular parasites. However, research over the past several years has revealed that the role of mast cells is not limited to IgE-mediated immune responses. Mast cells play a critical role in the pathogenesis of synovitis in a murine model of rheumatoid arthritis (RA). The synovium of patients with RA is chronically inflamed and characterized by an expanded population of MC, as in the mouse model. Mast cells are markedly increased in number and can make up 5% or more of the expanded population of total synovial cells. The number of accumulated MC differs substantially from patient to patient, in general varying directly with the intensity of joint inflammation. Mast cell mediators (histamine and tryptase) are also present at higher concentrations in the synovial fluid of inflamed human joints.

MC degranulation has long been associated with arthritis in several animal models, but a critical functional role in the disease was established in the K/B×N mouse model. This arthritis model closely mimics human RA via symmetric joint involvement, chronicity, a distal-to-proximal gradient of joint involvement, and histological features including synovial infiltrates, pannus, and erosions of cartilage and bone. Mice deficient in mast cells are highly resistant to arthritis, whereas reconstitution with normal mast cells restores the wild-type phenotype. Furthermore, degranulation of mast cells in the synovium is the first event observed histologically, occurring within 1-2 hours of administration of K/B×N serum. Thus, mast cells are a normal cell population within the human synovium and have a critical role in the pathogenesis of inflammatory arthritis.

Mast cells also mediate multiple sclerosis. Experimental allergic encephalomyelitis (EAE) is a rodent model of human multiple sclerosis (MS) characterized by inflammation in the central nervous system (CNS). Like the human disease, EAE is associated with an early breach of the blood-brain barrier, focal perivascular mononuclear cell infiltrates, and demyelination leading to paralysis of the extremities. While CD4-positive T cells have been implicated, the underlying cause of increased vascular permeability that facilitates the entry of T cells into the CNS is unknown.

Mast cell contribution to the pathogenesis of MS has been hypothesized based on their presence in CNS plaques of MS patients and the correlation between the number, distribution, or MC markers and MS or EAE pathology. Further evidence for past cells involvement in EAE/MS came from studies using mast cells-deficient mice. The mast cells-deficient W/Wv mice exhibited significantly reduced disease incidence, delayed disease onset, and decreased mean clinical scores when compared with their wild-type congenic littermates. No differences were observed in the T and B cell responses between the two groups and reconstitution of the mast cells population in W/Wv mice restores induction of early and severe disease to wild-type levels. These data provide a new mechanism for immune destruction in EAE and indicate that mast cells may be sentinels of neurologic inflammation.

### Build-Up of Arterial Plaque

“Arterial plaque” or “atherosclerosis” as used herein are interchangeable. In normal circumstances, the build-up of arterial plaque is a protective response to stresses on the endothelium and smooth muscle cells (SMCs) of the wall of the artery. In response to such stresses, atherosclerosis consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. For example, shear stresses are thought to be responsible for the frequent occurrence of atherosclerotic plaques in regions of the circulatory system where turbulent blood flow occurs, such as branch points and irregular structures.

The first observable event in the formation of an atherosclerotic plaque occurs when blood-borne monocytes adhere to the vascular endothelial layer and transmigrate through to the sub-endothelial space. Adjacent endothelial cells at the same time produce oxidized LDL. These oxidized LDL's are then absorbed in large amounts by the monocytes through scavenger receptors expressed on their surfaces. In contrast to the regulated pathway by which native LDL (nLDL) is absorbed by nLDL specific receptors, the scavenger pathway of uptake is not regulated by the monocytes. Oxidation of LDL into oxidized LDL results in the loss of the recognition of the apo B component by cellular LDL receptors, and in the preferential uptake of oxidized LDL by macrophage “scavenger” receptors. The enhanced endocytosis of oxidized LDL by vascular wall macrophages transforms them into lipid-laden foam cells that characterize early atherosclerotic lesions.

The lipid-filled monocytes are called foam cells, and are the major constituent of the fatty streak. Interactions between foam cells and the endothelial and SMCs which surround them lead to a state of chronic local inflammation which can eventually lead to smooth muscle cell proliferation and migration, and the formation of a fibrous plaque. Such plaques occlude the blood vessel concerned and thus restrict the flow of blood, resulting in ischemia.

Ischemia is a condition characterized by a lack of oxygen supply in tissues of organs due to inadequate perfusion. Such inadequate perfusion can have number of natural causes, including atherosclerotic or restenotic lesions, anemia, or stroke, to name a few. Many medical interventions, such as the interruption of the flow of blood during bypass surgery, for example, also lead to ischemia. In addition to sometimes being caused by diseased cardiovascular tissue, ischemia may sometimes affect cardiovascular tissue, such as in ischemic heart disease. Ischemia may occur in any organ; however, that is suffering a lack of oxygen supply.

The most common cause of ischemia in the heart is atherosclerotic disease of epicardial coronary arteries. By reducing the lumen of these vessels, atherosclerosis causes an absolute decrease in myocardial perfusion in the basal state or limits appropriate increases in perfusion when the demand for flow is augmented. Coronary blood flow can also be limited by arterial thrombi, spasm, and, rarely, coronary emboli, as well as by ostial narrowing due to luetic aortitis. Congenital abnormalities, such as anomalous origin of the left anterior descending coronary artery from the pulmonary artery, may cause myocardial ischemia and infarction in infancy, but this cause is very rare in adults. Myocardial ischemia can also occur if myocardial oxygen demands are abnormally increased, as in severe ventricular hypertrophy due to hypertension or aortic stenosis. The latter can be present with angina that is indistinguishable from that caused by coronary atherosclerosis. A reduction in the oxygen-carrying capacity of the blood, as in extremely severe anemia or in the presence of carboxy-hemoglobin, is a rare cause of myocardial ischemia. Not infrequently, two or more causes of ischemia will coexist, such as an increase in oxygen demand due to left ventricular hypertrophy and a reduction in oxygen supply secondary to coronary atherosclerosis. See, for example, U.S. Pat. No. 6,492,126 for additional information regarding atherosclerosis and ischemia.

Free radical “scavengers” such as vitamins A, E, C, and selenium are believed to react with oxidized LDLs and render them incapable of oxidation. The inhibitory action of these antioxidants thus inhibits the formation of oxidized LDL, thereby lowering the levels of arterial plaque deposits in blood vessels. See, for example, U.S. Pat. No. 6,326,031 for additional background regarding LDL, O-LDL, HDL, and arterial plaque.

Fullerenes effectively block the immune cascade that follows subcutaneous injection of phorbol myristate (PMA). Without wishing to be bound by theory, it is believed that a mechanism of action of this blockade may involve free radical scavenging. Membrane trafficking and permeability may be contributing to the biological response.

Peripheral blood monocytes, when placed in a tissue culture dish will adhere and become macrophages. However they do not normally ingest LDL added to the culture medium. Chemical modification of LDL, e.g., by oxidation, will stimulate macrophages to take up LDL. Another technique for stimulating human peripheral monocytes to ingest LDL is to incubate the macrophages with PMA, as shown by Kruth et al., J Biol Chem, 277:34573 (2002).

Without wishing to be bound by theory, it is believed that a compound that blocks PMA inflammatory response in skin could also block the same pathway in foam cells. Thus, a proposed intracellular mechanism for controlling the uptake of LDL is to use fullerenes to block the inflammatory mechanism in foam cells and thereby preventing these cells from accumulating lipids.

### Fullerenes

“Fullerene” or “fullerene molecule” generally refers to any member of the fullerene family of carbon cage molecules. Fullerenes are generally carbon structures formed of five and six membered rings arranged so that the rings form a closed geodesic sphere or spheroid held together by a combination of single and double carbon:carbon covalent bonds. The fullerenes in this disclosure can be defined by the formula: C₂ₛ wherein s is greater than or equal to 30, such as from about 30 to about 200 or from about 30 to about 100. For example, the fullerenes include C₆₀, C₇₀, and similar molecules that range in molecular weight from C₆₀ up to C₈₄, C₉₀, and larger such molecules, with shapes ranging from spheroidal to ellipsoidal, elongated and other shapes, and including not only single-walled but also multi-walled cages consisting of stacked or parallel layers. The fullerenes may be unmodified or underivatized. Alternatively, the fullerenes may enclose one or more atoms such as metal atoms, or other small chemical groups, inside the carbon cage; such fullerenes are sometimes called endohedral fullerenes. Fullerenes, as used herein, also include structures with chemical functional groups attached to the surface of the carbon cage. The functional groups can be covalently bound to the carbon cage via opening carbon:carbon double bonds. Fullerenes also include other structural variants, derivatives, and/or modified or functionalized fullerenes as described herein and/or as known in the art. The fullerenes can be synthetic or naturally-occurring. Synthetic fullerene molecules can be prepared in a laboratory by known methods (see, e.g., U.S. Pat. No. 5,177,248 and Krätschmer et al., Chem. Phys. Lett., 170, 167-170 (1990)) or can be purchased commercially.

In one embodiment, the fullerenes are water soluble, meaning the fullerenes distribute more or less uniformly in an aqueous solution and do not significantly precipitate. Water soluble fullerenes are known in the art as described above, and can be synthesized for example by attaching one or more hydrophilic chemical groups to the surface of the carbon cage. Suitable hydrophilic chemical groups include niacin group, hydroxyl or polyhydroxyl groups and N-ethylpolyamino groups. Non-limiting examples of water soluble fullerenes include C₆₀(OH)ₙ, C₆₀(NH—CH₂—CH₃)ₙ, and C₇₀-tetraglycolate. Many other examples of water-soluble fullerenes can involve the addition of one or more charged groups such as phosphates, sulfates, ammonium, carboxylates, or other charged groups; or hydrophilic, such as hydroxyl and polyhydroxyl groups; and carbohydrates, peptides, proteins, nucleotides and DNA.

In another embodiment, chemical groups such as amphiphilic or lipophilic groups can be attached to the carbon cage instead of or in combination with hydrophilic chemical groups.

“Fullerene,” “fullerene compound” or “fullerene derivative” as used herein refers to certain synthetically modified fullerene molecules as described herein, including synthetically modified fullerenes of the formula Zₘ—F—Yₙ. The fullerenes comprise closed cages of 60 to 200 carbon atoms which may also include chemical moieties attached to the exterior and/or incorporated within the cage.

Certain synthetically modified fullerene molecules are described in copending U.S. patent application Ser. No. 12/073,230, U.S. Patent Application Publication No. 2008-0213324-A1, filed Mar. 3, 2008, entitled “AMPHIPHILIC OR LIPOPHILIC POLAR FUNCTIONALIZED FULLERENES AND THEIR USES,” the entire disclosure of which is incorporated by reference herein.

The synthetically modified fullerene molecules as described in the copending application include fullerenes that have an aspect ratio ≠1, with an equatorial band and two opposing poles, and comprise an adduct at one or both poles.

In one embodiment, the synthetically modified fullerene has the formula

Zₘ—F—Yₙ,

wherein F is a fullerene of formula Cₚ or X@Cₚ, the fullerene having two opposing poles and an equatorial region;

Cₚ represents a fullerene cage having p carbon atoms, and X@Cₚ represents such a fullerene cage having a chemical group X within the cage.

Z and Y are positioned near respective opposite poles of Cₚ;

m is an integer of from 1 to 5 and Z is a hydrophilic, lipophilic, or amphiphilic chemical moiety;

n is an integer of from 1 to 5 and Y is a hydrophilic chemical moiety;

p is an even number between 60 and 200; and

X, if present, represents one or more metal atoms within the fullerene (F), optionally in the form of a trinitride of formula Gᵢ₌₁₋₃Hₖ₌₃₋ᵢN in which G and H are metal atoms. In one embodiment, at least one of G and H represents a rare earth element, a group IIIB element in the periodic table of elements or the like. Examples of suitable rare earth elements and group IIIB elements may include, but are not limited to, scandium (Sc), erbium (Er), holmium (Ho), yttrium (Y), lanthanum (La), gadolinium (Gd), thulium (Tm), dysprosium (Dy), terbium (Tb) and ytterbium (Yb).

In exemplary variations, p is an even number between 60 and 120, with p=60-96 being preferred, and p=60 or p=70 being more preferred. The synthetically modified fullerene can be arranged wherein each chemical moiety Z is composed of formula AᵣB in which A is a hydrophilic, lipophilic or amphiphilic chemical moiety, r is an integer of from 1 to 4, and B is a chemical linker connecting A to the fullerene, and each chemical moiety Y is composed of formula DE, in which E is a hydrophilic chemical moiety, v is an integer of from 1 to 4, and D is a chemical linker connecting the hydrophilic chemical moiety E to the fullerene.

In certain embodiments, B and/or E contain at least one —C(O)O— moiety. In certain embodiments, one or more A contains, at a free end thereof, a —(CH₂)_{q}CH₃ or —(OCH₂CH₂)_{w}OCH₃ moiety. q is an integer of from 3 to 25, preferably from 4 to 20, and more preferably from 5 to 17. w is an integer of from 1 to 12, preferably from 1 to 9, and more preferably from 1 to 6. In certain embodiments, A and/or D contain at least one niacin moiety at a free end thereof. In a preferred embodiment, the chemical moiety Y contains two niacin moieties. In another preferred embodiment, the chemical moieties Y and Z each contains two niacin moieties. In a further embodiment, Y has formula DE₂, wherein each E contains niacin moiety at the end thereof, and Z has formula A₂B, wherein each A contains niacin moiety at the end thereof.

The synthetically modified fullerene can be a prolate ellipsoid shaped fullerene having a major axis such that said poles are located at opposing ends of the major axis of the prolate ellipsoid fullerene. Alternatively, the fullerene can be spheroid with opposing poles defined by an axis through opposing carbon rings. Z and Y can configured such that when the molecule is contacted with a lipid bilayer in an aqueous medium, the equatorial region of F is selectively located within or in close proximity to the phospholipid bilayer. The molecule can be configured so that in an extended configuration has an aspect ratio of about 2.1 to 15, and a diameter less than about 2 nm. Such configurations are preferred configurations for incorporation of the molecules into lipid bilayers.

In another embodiment, the synthetically modified fullerene molecule has the formula Z(Cₚ)Y wherein: p is an even number between 60 and 200, preferably p=60 or 70; Y is a hydrophilic moiety covalently connected to Cₚ, optionally through a linking group, at or near a pole thereof; Z is a hydrophilic, lipophilic or amphiphilic moiety covalently connected to Cₚ, optionally through a linking group, at or near a pole opposite to Y; and Y is capable of anchoring the synthetic fullerene molecule to a lipid membrane.

In another embodiment, the synthetically modified fullerene molecule has the formula Z(Cₚ)Y wherein: p is an even number between 60 and 200, preferably p=60 or 70; Y is a hydrophilic moiety covalently connected to Cₚ, optionally through a linking group, at or near a pole thereof; Z is a hydrophilic moiety covalently connected to Cₚ, optionally through a linking group, at or near a pole opposite to Y; and Y is capable of anchoring the synthetic fullerene molecule to a lipid membrane.

In another embodiment, the synthetically modified fullerene molecule has the formula Z(C₇₀)Y; wherein Y is a hydrophilic moiety covalently connected to C₇₀, optionally through a linking group, at or near a pole thereof; Z is a hydrophilic, lipophilic or amphiphilic moiety covalently connected to C₇₀, optionally through a linking group, at or near a pole opposite to Y; and Y is capable of anchoring the synthetic fullerene molecule to a lipid membrane.

In another embodiment, the synthetically modified fullerene molecule has the formula Z(C₇₀)Y wherein: Y is a hydrophilic moiety covalently connected to Cₚ, optionally through a linking group, at or near a pole thereof; Z is a hydrophilic moiety covalently connected to Cₚ, optionally through a linking group, at or near a pole opposite to Y; and Y is capable of anchoring the synthetic fullerene molecule to a lipid membrane.

In another embodiment, the synthetically modified fullerene molecule has the formula Zₘ—F—Yₙ wherein:

F is a fullerene of formula Cₚ having 60-200, preferably 60 or 70, carbon atoms;

m is an integer of from 1 to 5 such that each Z is a group AᵣBₛ in which r is an integer of from 1 to 4, s is an integer of from 1 to 4, and A is one or more hydrophilic or polar group bonded to the fullerene through one or more linker B;

n is an integer of from 1 to 5 and each Y is a group DₜEᵥ in which t is an integer of from 1 to 4, v is an integer of from 1 to 4 and E is one or more hydrophilic group bonded to the fullerene through one or more linker D; and,

X and Y are positioned at or near opposite poles of F.

In certain embodiments, the synthetically modified fullerene has a geometrical configuration capable of causing the fullerene molecule to locate within phospholipid bilayers of a cell such that a radical scavenging zone near the equatorial band of the fullerene is situated within or in close proximity to the phospholipid bilayer.

A plurality of such synthetically modified fullerene molecules can be uniformly dispersed in phospholipids, such as in liposomes. The amphipathic fullerene molecules described herein do not generally form vesicles by themselves, but require membrane-forming phospholipids in mole ratios greater than 1:1 (lipid:fullerene adduct) to form vesicles.

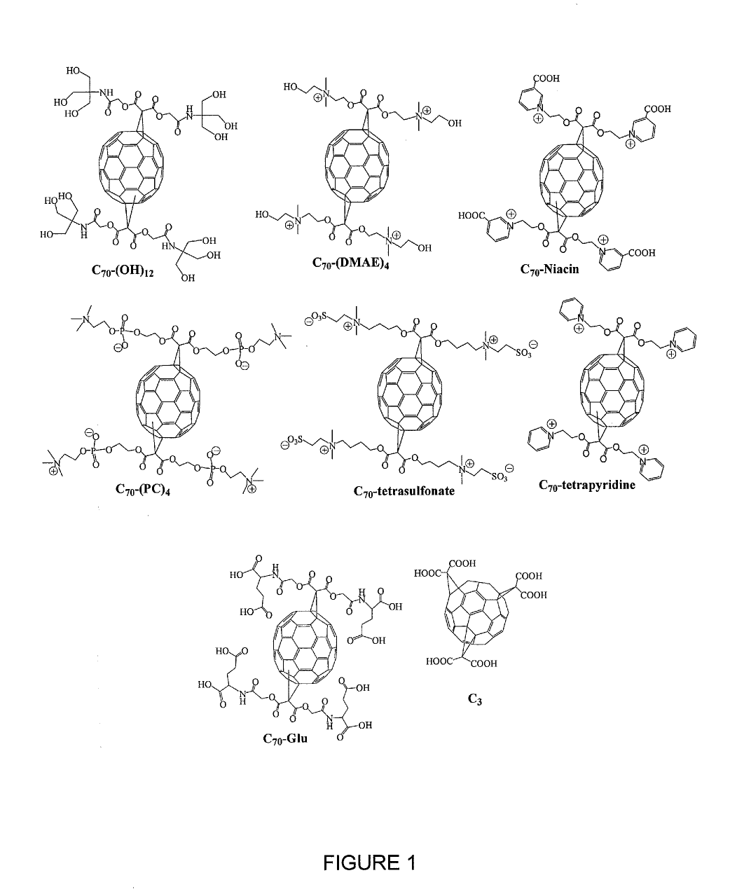

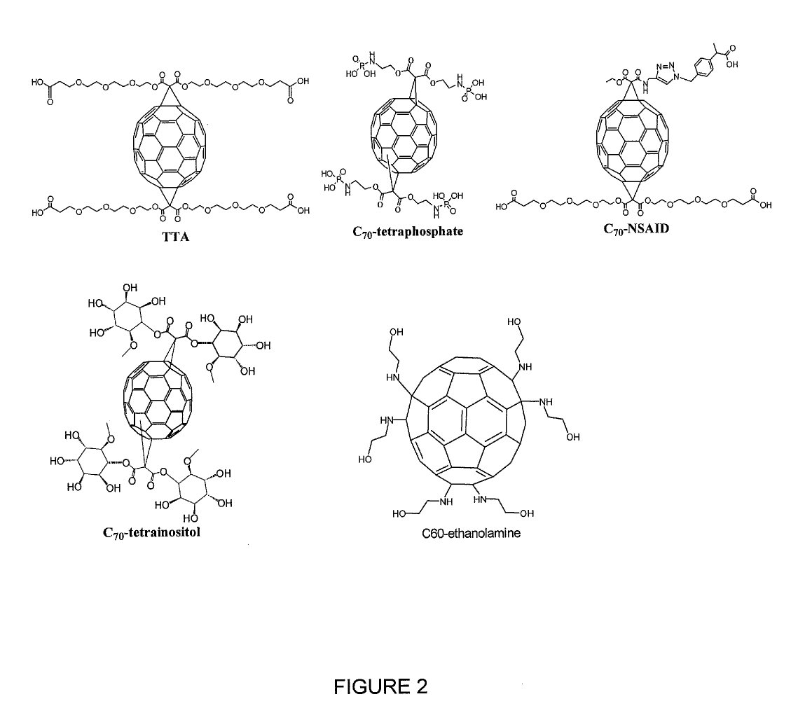

In exemplary embodiments, the fullerene comprises any one or more of compounds shown in FIGS. 1-4.

Suitable fullerenes are also described in the following co-pending U.S. application Ser. No. 12/921,106, filed Sep. 3, 2010, which is a national stage of PCT Application No. PCT/US2009/001334, filed on Mar. 3, 2009, published as WO 2009/114089, entitled “USING FULLERENES TO ENHANCE AND STIMULATE HAIR GROWTH;” U.S. application Ser. No. 12/921,072, filed Sep. 3, 2010, which is a national stage of PCT/US2009/001332, filed on Mar. 3, 2009, published as WO 2009/114087, entitled “METHOD FOR TREATING PRURITUS BY ADMINISTERING FULLERENES;” U.S. application Ser. No. 12/921,143, filed Sep. 3, 2010, which is a national stage of PCT/US2009/001329, filed on Mar. 3, 2009, published as WO 2009/114084, entitled “METHOD FOR TREATING WOUNDS BY ADMINISTERING FULLERENES;” and Sarah K. Norton, Anthony Dellinger, Zhiguo Zhou, Robert Lenk, Darren MacFarland, Bechy Vonakis, Daniel Conrad, and Christopher L. Kepley, A new class of human mast cell and peripheral blood basophil stabilizers that differentially control allergic mediator release, Clin. Transl Sci. 2010 August: 3(4): 158-169, the entire disclosure of each of which are incorporated herein by reference.

“Pharmaceutically acceptable salt” refers to pharmaceutically acceptable salts of fullerenes which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The terms “inhibiting”, “treating,” or “treatment” and the like are used herein to generally mean obtaining a desired pharmacological and physiological effect, and refer to complete elimination as well as to any clinically or quantitatively measurable reduction in the condition for which the subject is being treated. “Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. More specifically, the fullerenes described herein are used to treat a subject with an inflammatory disorder or to inhibit the build-up of arterial plaque in a subject. These fullerenes are provided in a therapeutically effective amount to: prevent the disorder (i.e., inhibit the onset or occurrence of the disorder and/or cause the clinical symptoms of the disorder not to develop in a mammal that may be exposed to or predisposed to the disorder but does not yet experience or display symptoms of the disorder); inhibit the disorder (i.e., arrest or reduce the development of the disorder or its clinical symptoms); or relieve the disorder (i.e., cause regression of the disorder or its clinical symptoms). Subjects in need of treatment include those already with one or more inflammatory disorder as well as those in which one or more inflammatory disorder is to be prevented, and all subjects in whom the inhibition of the build-up of arterial plaque is desired.

A “subject in need thereof” refers to any subject or individual who could benefit from the method of treatment described herein. In certain embodiments, a subject in need thereof is a subject predisposed for the development of one or more inflammatory disorders; a subject having one or more inflammatory disorders but not exhibiting any clinical symptoms; or a subject having one or more inflammatory disorders and suffering from the symptoms of the one or more iron inflammatory disorders. The “subject in need thereof” refers to a vertebrate, such as a mammal. Mammals include, but are not limited to, humans, other primates, rodents (i.e., mice, rats, and hamsters), farm animals, sport animals and pets. In one embodiment, the subject is a mammal such as a human. In certain embodiments, the methods find use in experimental animals, in veterinary application, and/or in the development of animal models for disease.

As used herein, the term “administering” or “introducing” a fullerene to a subject means providing the fullerene to a subject. Methods of administering fullerenes to subjects include any of a number of convenient means including, but not limited to, systemic administration (e.g. intravenous injection, intraparenteral injection, inhalation, transdermal delivery, oral delivery, nasal delivery, rectal delivery, etc.) and/or local administration (e.g. direct injection into a target tissue, delivery into a tissue via cannula, delivery into a target tissue by implantation of a time-release material, or delivery through the skin via a topical composition such as a cream, lotion, or the like), delivery into a tissue by a pump, etc., intraosseously, in the cerebrospinal fluid, or the like. “Orally delivery” refers to administration in an oral form, such as in a pharmaceutically acceptable carrier and/or diluent. Oral delivery includes ingestion of the drug as well as oral gavage of the drug. Further modes of administration include buccal, sublingual, vaginal, subcutaneous, intramuscular, or intradermal administration.

Modes of administration can include delivery via a sustained release and/or controlled release drug delivery formulation and/or device. “Sustained release” refers to release of a drug or an active metabolite thereof into the systemic circulation over a prolonged period of time relative to that achieved by oral administration of a conventional formulation of the drug. “Controlled release” is a zero order release; that is, the drug releases over time irrespective of concentration. Single, multiple, continuous or intermittent administration can be effected.

In one embodiment, a composition comprising fullerenes is administered orally to a subject having an inflammatory arthritis such as rheumatoid arthritis. In another embodiment, a composition comprising fullerenes is injected directly into an affected joint of a subject having an inflammatory arthritis such as rheumatoid arthritis. In yet another embodiment, a composition comprising fullerenes is administered via a topical formulation applied to the skin proximal to an affected joint of a subject having an inflammatory arthritis such as rheumatoid arthritis.

In some embodiments, a pharmaceutical composition or formulation comprising plaque targeted fullerenes is administered orally to a subject in whom the inhibition of the build-up of arterial plaque is desired. These fullerenes are substantially absorbed in the intestine and become incorporated into LDL particles in the liver such that a therapeutically effective amount of fullerenes is delivered to the foam cells and the fullerenes block further accumulation of LDL into plaque.

In another embodiment, a composition comprising fullerenes is injected directly into the vasculature of a subject in whom the inhibition of the build-up of arterial plaque is desired, such that a therapeutically effective amount of fullerenes are absorbed by arterial plaque to block further accumulation of LDL into arterial plaque. In yet another embodiment, a composition comprising cholesterol modified fullerenes is administered directly to vasculature wherein such cholesterol modified fullerenes form micelles which partition into LDL particles within the vasculature such that a therapeutically effective amount of the fullerenes is absorbed by arterial plaque to block further accumulation of LDL into arterial plaque. Targeting of foam cells in arterial plaque is accomplished through the attachment of groups (i.e., cholesterol derivatives) which home to cholesterol receptors on the foam cells.

“Optional” or “optionally” means that the subsequently described event or circumstance may, but need not, occur, and that the description includes instances where the event or circumstance occurs and instances in which it does not.

As used herein, “pharmaceutical composition” and “pharmaceutical formulation” are interchangeable.

A “therapeutically effective amount” or “pharmaceutically effective amount” means the amount of a fullerene that, when administered to a subject for treating an inflammatory disorder, or to a subject in whom the inhibition of the build-up of arterial plaque is desired, is sufficient to effect such treatment for the disorder or desired inhibition of the build-up of arterial plaque. Thus, a “therapeutically effective amount” is an amount indicated for treatment while not exceeding an amount which may cause significant adverse effects. The “therapeutically effective amount” will vary depending on the type of fullerene to be administered, and will also be determined by physical and physiological factors such as the disorder and its severity or the degree of inhibition of the build-up arterial plaque desired, and the age, body weight, and/or clinical history of the subject to be treated. Methods for evaluating the effectiveness of therapeutic treatments are known to those of skill in the art.

Doses to be administered are variable according to the treatment period, frequency of administration, the host, and the nature and severity of the disorder. The dose can be determined by one of skill in the art without an undue amount of experimentation. The fullerenes are administered in dosage concentrations sufficient to ensure the release of a sufficient dosage unit into the patient to provide the desired treatment of the inflammatory disorder or the desired level of inhibition of the build-up of arterial plaque. The actual dosage administered will be determined by physical and physiological factors such as age, body weight, severity of condition, and/or clinical history of the patient. In some embodiments, the active ingredients may be administered to achieve therapeutic or prophylactic blood concentrations, such as in vivo plasma concentrations of the fullerenes of from about 0.01 to about 10,000 ng/cc, such as from about 0.01 to about 1,000 ng/cc. “Therapeutic or prophylactic blood concentrations” refers to systemic exposure to a sufficient concentration of a drug or an active metabolite thereof over a sufficient period of time to effect disease therapy or to prevent the onset or reduce the severity of a disease in the treated animal.

For example, the methods described herein may use compositions to provide from about 0.01 to about 100 mg/kg body weight/day of the fullerenes, from about 0.01 to about 10 mg/kg body weight/day of the fullerenes, or about 30 mg/kg body weight/day of the fullerenes. It will be understood, however, that dosage levels that deviate from the ranges provided may also be suitable in the treatment of a given disorder.

The fullerenes may be in any form suitable for administration. Such administrable forms include tablets, buffered tablets, pills, capsules, enteric-coated capsules, dragees, cachets, powders, granules, aerosols, liposomes, suppositories, creams, lotions, ointments, skin patches, parenterals, lozenges, oral liquids such as suspensions, solutions and emulsions (oil-in-water or water-in-oil), ophthalmic liquids and injectable liquids, or sustained- and/or controlled release forms thereof. The desired dose may be provided in several increments at regular intervals throughout the day, by continuous infusion, or by sustained and/or controlled release formulations, or may be presented as a bolus, electuary or paste.

“Practical dosage regimen” refers to a schedule of drug administration that is practical for a patient to comply with. For human patients, a practical dosage regimen for an orally administered drug is likely to be an aggregate dose of less than 10 g/day.

In one embodiment, a pharmaceutical composition or formulation comprising the fullerenes is prepared by admixture with one or more pharmaceutically acceptable carriers and/or excipients. Other active ingredients and/or additives may be added, if desired, to maximize fullerene preservation, to optimize a particular method of delivery, or to optimize the desired effects in the subject to be treated. In addition, according to certain embodiments, the pharmaceutical composition or formulation includes use of combination compositions comprising the fullerenes as described herein in combination with other agents suitable for the treatment of inflammatory disorders or for the inhibition of the build-up of arterial plaque.

The fullerenes may be formulated into a variety of compositions (i.e., formulations or preparations). These compositions may comprise any component that is suitable for the intended purpose, such as conventional physiologically acceptable delivery vehicles, diluents and excipients including isotonising agents, pH regulators, solvents, solubilizers, dyes, gelling agents and thickeners and buffers and combinations thereof. Pharmaceutical formulations suitable for use with the instant fullerenes can be found, for instance, in Remington's Pharmaceutical Sciences. Physiologically acceptable carriers are carriers that are nontoxic at the dosages and concentrations employed. Pharmaceutical formulations herein comprise pharmaceutical excipients or carriers capable of directing the fullerenes to the area where the subject in need thereof is a subject in whom the inhibition of the build-up of arterial plaque is desired. Suitable excipients for use with fullerenes include water, saline, dextrose, glycerol and the like.

In various embodiments, the fullerenes are administered to a subject in need thereof in the form of pharmaceutical compositions or formulations. These pharmaceutical compositions or formulations comprise fullerenes and can also include one or more pharmaceutically acceptable carriers or excipients. The excipient is typically one suitable for administration to human subjects or other mammals. In making the compositions of this disclosure, the active ingredient (i.e., fullerenes) is usually mixed with an excipient, and/or diluted by an excipient. When the excipient serves as a diluent, it can be a solid, semi-solid, or liquid material, which acts as a vehicle, carrier or medium for the active ingredient. For additional information regarding suitable methods and formulations for use in the present disclosure are found in REMINGTON'S PHARMACEUTICAL SCIENCES, Mace Publishing Company, Philadelphia, Pa., 17th ed. (1985).

According to one embodiment, the fullerenes may be administered alone, or in combination with any other medicament. Thus, the formulation may comprise fullerenes in combination with another active ingredient, such as a drug, in the same formulation. When administered in combination, the fullerenes may be administered in the same formulation as other compounds as shown, or in a separate formulation. When administered in combination, the fullerenes may be administered prior to, following, or concurrently with the other compounds and/or compositions.

In certain embodiments, the pharmaceutical compositions or formulations described herein have a viscosity at 20° C. of from about 5 cps to about 50000 cps, such as from about 500 cps to about 40000 cps, or about 5000 cps to about 30000 cps.

Preparation of dry formulations that are reconstituted immediately before use also is contemplated. The preparation of dry or lyophilized formulations can be effected in a known manner, conveniently from the solutions of the invention. The dry formulations of this invention are also storable. By conventional techniques, a solution can be evaporated to dryness under mild conditions, especially after the addition of solvents for azeotropic removal of water, typically a mixture of toluene and ethanol. The residue is thereafter conveniently dried, e.g., for some hours in a drying oven.

The fullerene-containing preparations described above may be administered systemically or locally and may be used alone or as components of mixtures. In one embodiment the administration is local. The route of administration for the fullerenes may be intravenous, oral, or by use of an implant.

Additional routes of administration are subcutaneous, intramuscular, or intraperitoneal injections of the fullerenes in conventional or convenient forms.

Generally, the pharmaceutical compositions or formulations described herein can be administered as a pharmaceutical or nutritional formulation. These compositions or formulations can be administered orally, intravenously, or as a suppository.

“Pharmaceutically acceptable carrier” or “diluent” means a carrier that is useful in preparing a pharmaceutical composition that is generally safe, neither biologically nor otherwise undesirable, not toxic or otherwise unacceptable commensurate with a reasonable benefit/risk ratio, compatible with other ingredients of the formulation, and includes a carrier that is acceptable for veterinary use as well as human pharmaceutical use. “A pharmaceutically acceptable carrier” as used in the specification and claims includes both one and more than one such carrier.

A “pharmaceutically acceptable carrier” includes any and all solvents, dispersion media, coatings, antibacterial and anti-fungal agents, isotonic and absorption delaying agents, and the like, compatible with pharmaceutical administration of a composition comprising fullerenes. Examples of such carriers or diluents include, but are not limited to, water, saline, Ringer's solutions and dextrose solution. The volume of the pharmaceutical composition is based on the intended mode of administration and the safe volume for the individual patient, as determined by a medical professional.

The selection of carrier also depends on the intended mode of administration. Fullerenes of the present invention may be administered by any of a number of convenient means including, but not limited to systemic administration (e.g., intravenous injection, intraparenteral injection, inhalation, transdermal delivery, oral delivery, nasal delivery, rectal delivery, etc.) and/or local administration (e.g., direct injection into a target tissue, delivery into a tissue via cannula, delivery into a target tissue by implantation of a time-release material, or delivery through the skin via a topical composition such as a cream, lotion, or the like), delivery into a tissue by a pump, etc., orally, parenterally, intraosseously, in the cerebrospinal fluid, or the like. Further modes of administration include buccal, sublingual, vaginal, subcutaneous, intramuscular, or intradermal administration.

### Fullerene Derivatives in a Murine Arthritis Model

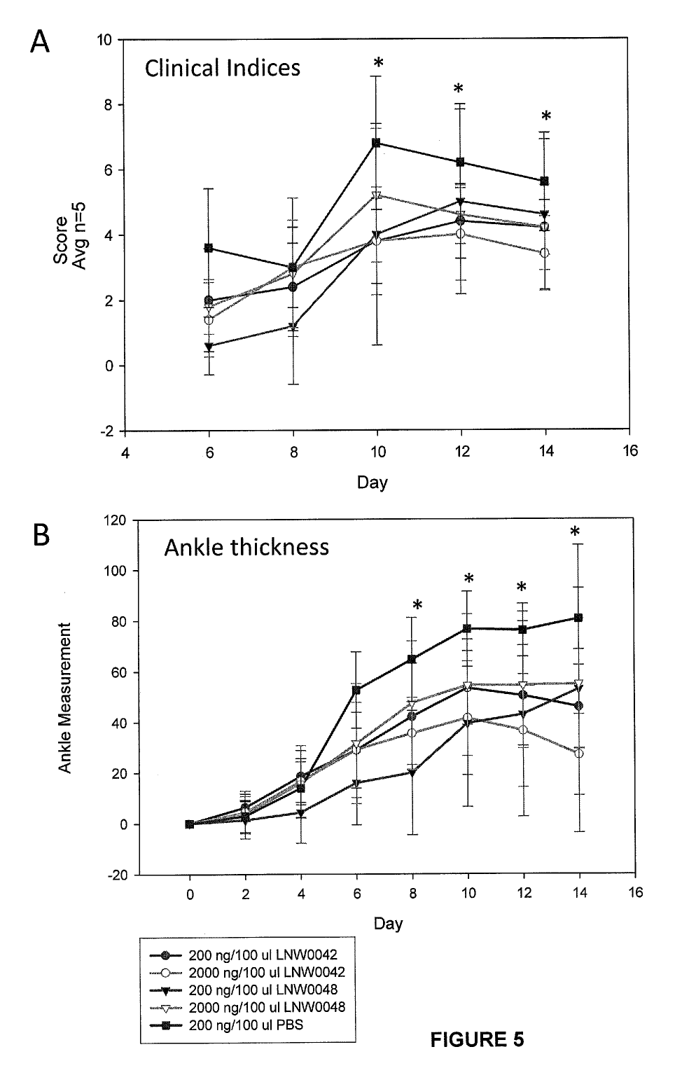

To induce disease, C57/B6 (5 mice/cage/group) mice were injected intraperitoneally (IP) on Days 1 and 3 with 100 μl of arthritogenic serum. Fullerene derivatives (200-2000 ng/100 μl phosphate buffered saline (PBS)) were injected IP on Day 0, 2, and every 2nd day thereafter. As a control 100 μl of PBS without serum was injected in the control group. Swelling in each ankle was measured along with the clinical indices as described (Lee, D M, Science 2002, Sep. 6; 297(5587):1689-92). Measurements were performed every second day by personnel blinded to the identity of the injections. After 14 days mice were sacrificed and ankle sections removed for histology and serum obtained for cytokine analysis. Error bars, SEM. In FIG. 5A, clinical index was given as the sum of observed inflammation (per paw): 0=no evidence of inflammation; 1=subtle inflammation (metatarsal phalanges joints, individual phalanx, or localized edema); 2=easily identified swelling but localized to either dorsal or ventral surface of paw; and 3=swelling on all aspects of paw. Maximum score=12. In FIG. 5B, the sum of the measurement in ankle swelling at each day was given. The * indicates significant differences observed on that day in fullerene compared to non-fullerene-treated mice.

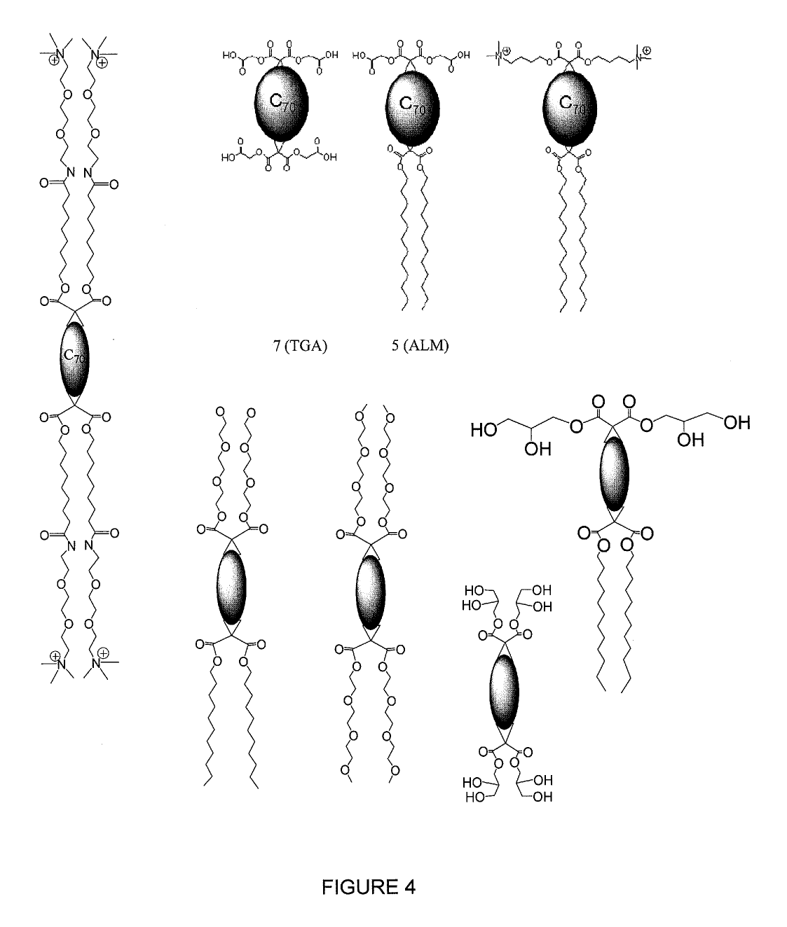

In FIG. 5, LNW0042 refers to compound 5 (see, e.g., FIG. 4), and LNW0048 refers to compound 7 (see, e.g., FIG. 4).

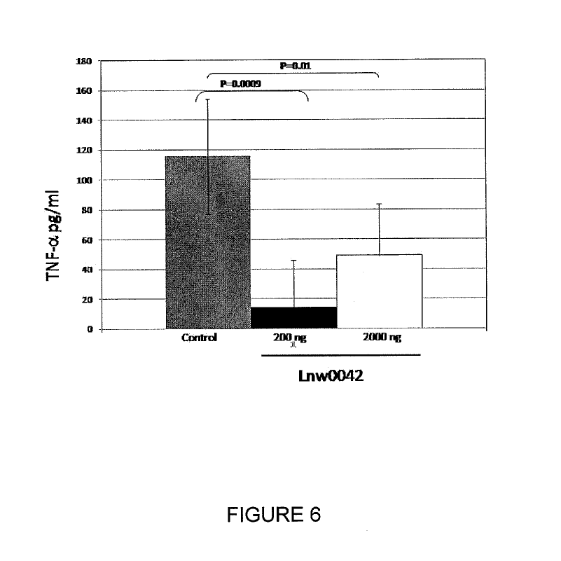

Tumor necrosis factor-alpha (TNF-α) is a major mediator of inflammatory arthritis. Several clinical trials have shown that TNF-α blocking agents, such as etanercept (co-marketed by Amgen and Wyeth under the trade name ENBREL®); infliximab (marketed under the trade name REMICADE® by Centocor); and adalimumab (marketed as HUMIRA® by Abbott Laboratories, Illinois, U.S.A.), significantly reduce the morbidity associated with inflammatory arthritis. As seen in FIG. 6, TNF-α in the serum at day 14 was significantly inhibited in the mice treated with fullerene derivatives. These results demonstrate that fullerene derivatives can inhibit inflammatory arthritis, possibly through the inhibition of TNF-α.

Prior to injection, 5 was incorporated into liposomes with egg phosphatidylcholine (PC) at a ratio of 1:2. 7 was dissolved in PBS buffer at pH 7.4.

The mouse arthritis model is characterized by the development of disease with many of the features of rheumatoid arthritis in humans. As seen in FIGS. 5 and 6, different fullerene formulations inhibited inflammatory arthritis. After arthritogenic serum transfer, PBS-treated mice exhibited typical clinical arthritis as determined using clinical indices and quantitative ankle swelling measurements. In contrast, mice treated with fullerene derivatives demonstrated a significant inhibition in both the clinical indices as well as ankle swelling measurements. Inflammation was significantly inhibited (p<0.04) by 5 from day 6 to 14. At day 14, concentrations of 200 ng/100 μl or 2000 ng/100 μl both had p<0.0001. In addition, at 200 ng/100 μl 7, inflammation was significantly inhibited from days 6 to 12 (p<0.03) and 2000 ng/100 μl 7, inflammation was significantly inhibited from days 10 to 14 (p<0.02).

### Inhibition of FcεRI-Mediated MC/PBB Activation

Fullerene derivatives were tested in different concentrations for their ability to inhibit FcεRI-mediated MC/PBB activation. Early activation events (ROS, Ca²⁺, and phosphorylation of signaling molecules) and later events (gene expression by microarray and quantification of those FcεRI-activated signaling molecules most affected by fullerene derivatives pre-treatment) were examined. In vivo inhibition of MC-dependent anaphylaxis was also analyzed. See, e.g., Sarah K. Norton, Anthony Dellinger, Zhiguo Zhou, Robert Lenk, Darren MacFarland, Bechy Vonakis, Daniel Conrad, and Christopher L. Kepley, A new class of human mast cell and peripheral blood basophil stabilizers that differentially control allergic mediator release, Clin. Transl Sci. 2010 August: 3(4): 158-169.

### Toxicity of Fullerene Derivatives

Fullerene derivatives were tested for cell toxicity by incubation with increasing concentrations up to 100 μg/ml and viability counts taken on days three, six, and nine. No toxicity was observed with any of fullerene derivatives compared to control cells (not shown).

### MC/PBB Fullerene Derivative Culture and FcεRI-Mediated Activation

MC are a component of the inflammatory response. As such, cultures of human Mast Cells can be used for screening the activity of different fullerene derivatives to evaluate their potential activity in animal models. In this example, human skin tissue was received from the Cooperative Human Tissue Network and MC purified and cultured as described in Kepley, C. L. Int. Arch. Allergy Immunol. 138, 29-39 (2005). The MC were cultured in media containing stem cell factor which is removed from the culture 24 hours prior to experimentation.

PBB were obtained from two sources: donors recruited under an IRB-approved protocol after informed consent or from leukopheresis packs obtained from the Johns Hopkins Hemapheresis Center. PBB were purified to ≧99% purity as described in Vonakis, B. M., Gibbons S Jr, Sora, R., Langdon, J. M., and MacDonald, S. M. J. Allergy Clin. Immunol. 108, 822-831 (2001), and Miura, K., Saini, S. S., Gauvreau, G., and MacGlashan, D. W., Jr. J. Immunol. 167, 2282-2291 (2001). Purified PBB were incubated overnight (20 hours) with a fullerene derivative (5 μg/ml) or vehicle control and a minimal (non-stimulatory) concentration of IL-3 (2 pg/ml) to prevent apoptosis. The next day, cells were washed and aliquoted for the histamine release assay by treatment with 0.1 mg/ml of goat polyclonal anti-IgE, buffer alone (spontaneous release) or perchloric acid (total histamine determination). Histamine was quantified in cell free supernatants using automated fluorimetry in duplicate. In a second set of experiments the two fullerene derivatives (at 5 μg/ml) were incubated with PBB for 20 hours, washed cells stimulated with 15 ng/ml anti-IgE for 18 hours in duplicate and supernatants collected for quantification of IL-13 by in-house ELISA. The optimal doses of anti-IgE are chosen for activation of PBB.

For activation, MC were suspended in fresh medium (without cytokines) and incubated for 16 hours with or without fullerene derivatives at 37° C. in a 6% CO₂ incubator. The 16 hour time point was chosen based on preliminary experiments demonstrating this was optimal for inhibition of mediator release (not shown) and uptake within FcεRI cells. The next morning, cells were washed and stimulated with anti-FcεRI Abs (3B4; 1 μg/ml) for 30 minutes (β-hexosaminidase) or overnight (GM-CSF) at 37° C. in a 6% CO₂ incubator and mediator release measured as described in Zhao, W., Kepley, C. L., Morel, P. A., Okumoto, L. M., Fukuoka, Y., and Schwartz, L. B. J Immunol. 177, 694-701 (2006). All MC mediator release studies were performed in triplicate.

### Western Blotting and Phospho-Signaling Quantification

Cell lysate preparation and Western blotting were performed using a protocol optimized for extracting phospho-proteins from human MC. Tkaczyk, C., Metcalfe, D. D., and Gilfillan, A. M. J. Immunol. Methods 268, 239-243 (2002). Following activation, cells were lysed directly in boiling denaturing sample buffer consisting of tris-buffered saline with triton-X-100 (0.5%) and protease and phosphatase inhibitors. The cell suspension was then passed through a 20-gauge needle, boiled, and centrifuged to remove cell debris. Proteins were separated on 8% or 10% NuPage Tris-Bis gels using Licor running buffer. Western blotting was performed using Licor blocking buffer and IR800 and IR700 anti-rabbit F(ab)₂ secondary Abs (1:1000). Primary Abs were from Cell Signaling Technologies or Santa Cruz unless otherwise noted. Band intensities were captured using the Odyssey Imaging System and bands quantified by measuring the number of pixels in each band using a box drawn for the same area of measurement for each separate blot. The band intensity was then normalized for loading by dividing the number of pixels in each band with the housekeeping band intensity (β-actin) performed on the same blot.

### Calcium and Reactive Oxygen Species (ROS) Measurement

MC were pre-treated with or without fullerene derivatives as above, washed with Tyrodes buffer supplemented with BSA, and incubated with Fura-2 AM (2 μM) for 30 minutes at 37° C. Cells were washed, stimulated with anti-FcεRI, and calcium flux measured in real time on a Perkin Elmer LS55 Spectrofluorometer. For ROS production, cells were exposed to fullerene derivatives as above. After washing, cells were re-suspended in X-Vivo medium containing 5 μM dichlorodihydrofluorescein (DCF) at 37° C. for 30 minutes, washed, and activation-induced changes in mean fluorescence was measured with excitation at 502 nM and emission at 523 nM for 15 minutes (19). The data is presented as fluorescence intensity of the 523 nM emission over time. All experiments were performed in triplicate and degranulation was measured in parallel. Separate experiments were performed to ensure that the fullerene derivatives do not interfere with indicator dye binding (not shown).

### Gene Microarray Studies and Validation Using Western Blotting or Flow Cytometry

Mast cells (1×10⁷ cell/condition; each condition performed in triplicate) were incubated with or without fullerene derivatives as above and incubated with or without anti-FcεRI antibodies for 10 minutes, supernatants were removed (to remove pre-formed mediators), and fresh warm medium containing anti-FcεRI antibodies (14 ml) added for 2 hours. Cells were centrifuged, the supernatant and the pellet immediately frozen and microarray performed using the Human Whole Genome OneArray™ gene expression profiling service (Phalanx Biotech Group). Separate samples, assayed in parallel were lysed and protein expression analyzed by Western blotting or flow cytometry (CD45) as described in Kepley, C. L., Pfeiffer, J., Wilson, B. W., Schwartz, L. B., and Oliver, J. M. J. Leukocyte Biol. 64, 474-483 (1998).

For microarray RNA was isolated using the Ambion MessageAmp aRNA kit; all samples passed the internal quality control checks. For hybridization each sample was run in triplicate. Optical density was measured by NanoDrop ND-1000. The ratio of absorbance at 260 nm and 280 nm provides an estimate of RNA purity. Samples were found to have ratios between 1.8 and 2.2 indicating highly pure samples. The reactive amino group of 5-(3-aminoallyl)-UTP/5-(3-aminoallyl)-dUTP was used to conjugate the purified aRNA/cDNA with the NHS-CyDye. Labeling efficiency was calculated by the concentration of CyDye and aRNA/cDNA which was above 10. For hybridization 10 μg Cy5-labeled aRNA was utilized by the Phalanx Hybridization Protocol Array Version HOA 4.3.

Pearson correlation tables (R values) for each technical repeat were calculated from raw log 2 intensity (R) and normalized log 2 intensity (N) values and compared to each other. Only probes with P value (detected) less than 0.05 were included in the calculation. This analysis showed good correlation between platforms when filtered stringently for fold change and loosely for significance (p-value). Greater than 95% of the samples had R values of 0.8 or greater indicating strong correlation between the two parameters.

In order to examine the genes most affected by fullerene derivative preincubation the resulting values were sorted and filtered the following way. First, the mean (±SD) of the six values from each condition for each of the 30,970 genes was calculated. Second, only normalized value intensities of >100 were included so that follow up detection of protein levels would be more likely to be successful. Third, these data were further truncated to include only normalized data in which the average FcεRI activation (without pre-treatment) significantly (P<0.05) increased at least 10% compared to non-FcεRI resting cells. Fourth, the genes with >10% upregulation by FcεRI stimulation alone were compared to those samples pretreated with each fullerene derivative and only those showing at least >20% inhibition or >20% upregulation (non-treated+anti-FcεRI compared to treated+anti-FcεRI) were examined. Data are presented as the average percentage downregulated with fullerene derivative treatment derived from the following equation:

$$\frac{[ \begin{matrix}( Non \text{-} treated + Fc ɛ RI activation ) - \\ ( FD \text{-} treated + Fc ɛ RI activation )\end{matrix} ]}{Non \text{-} treated + Fc ɛ RI activation}$$

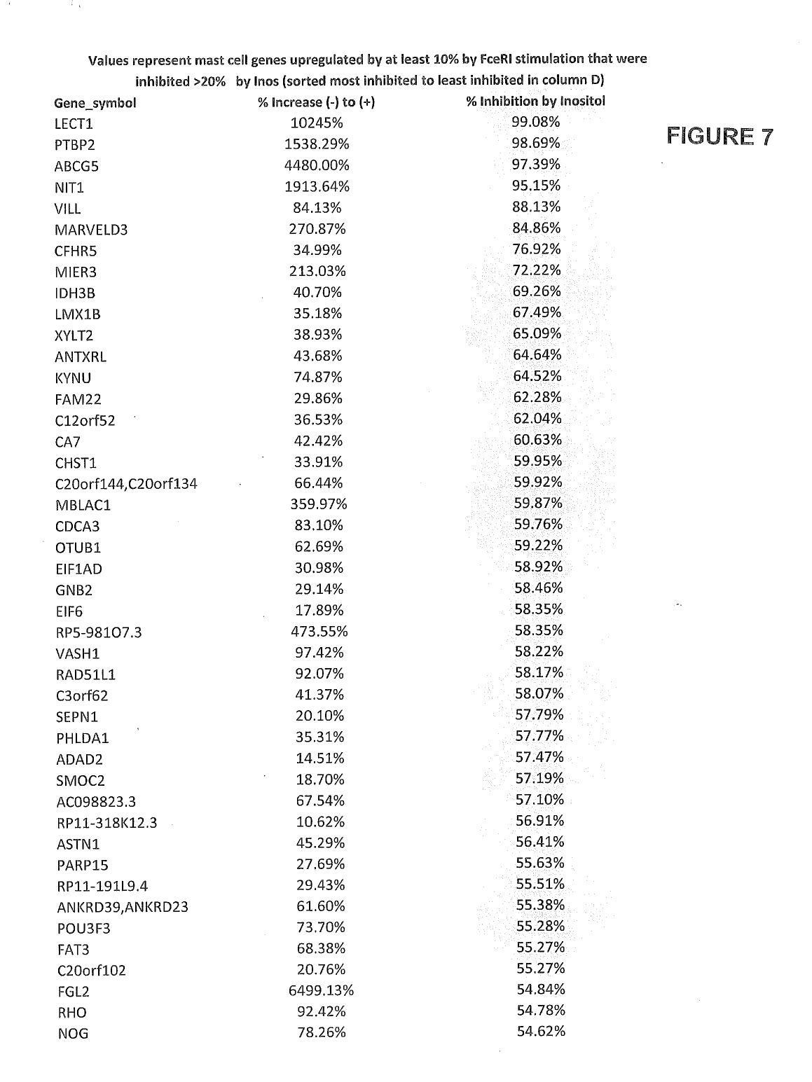

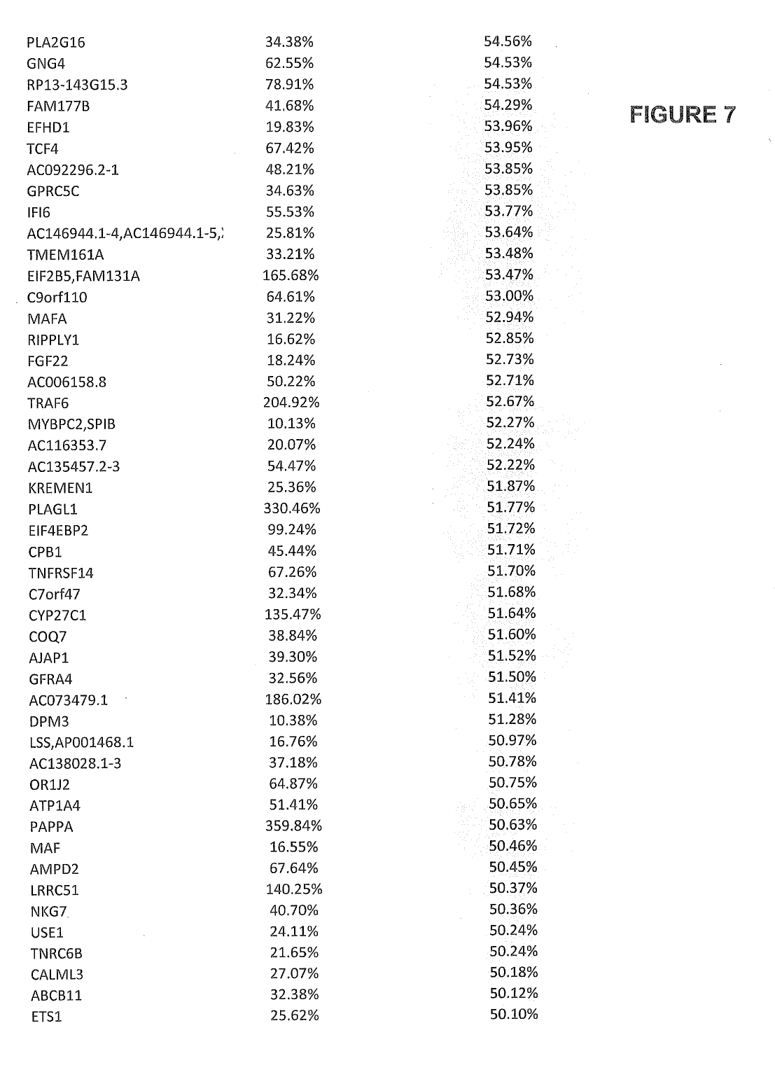

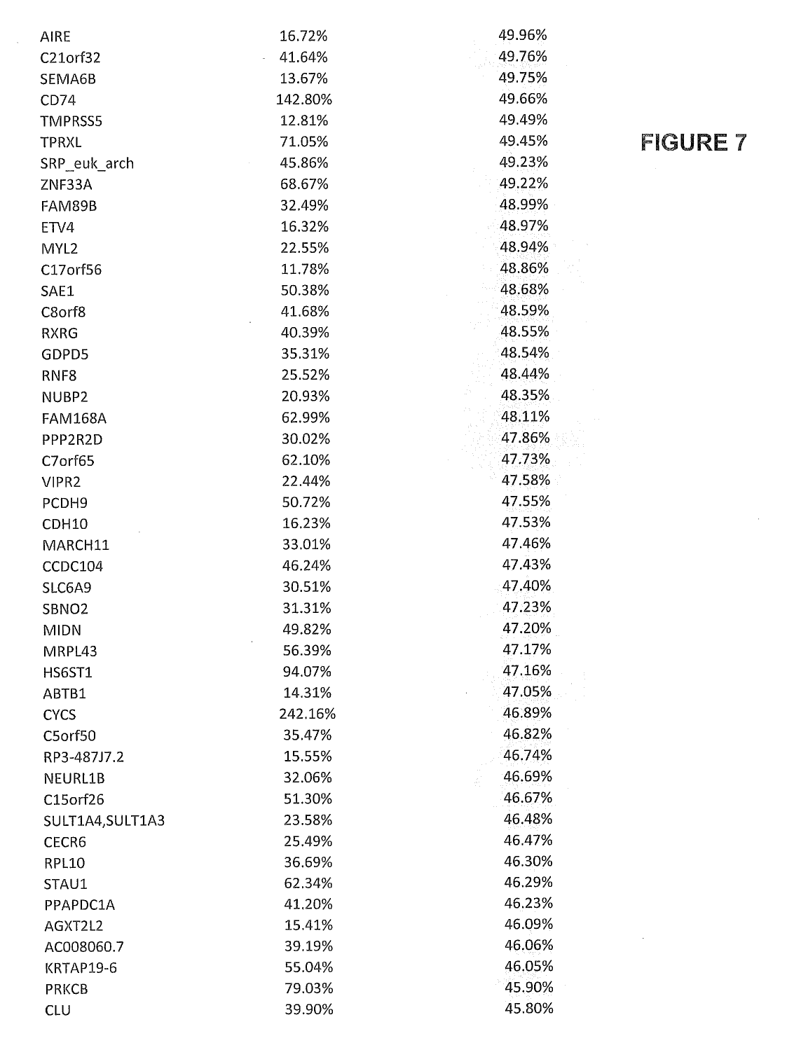

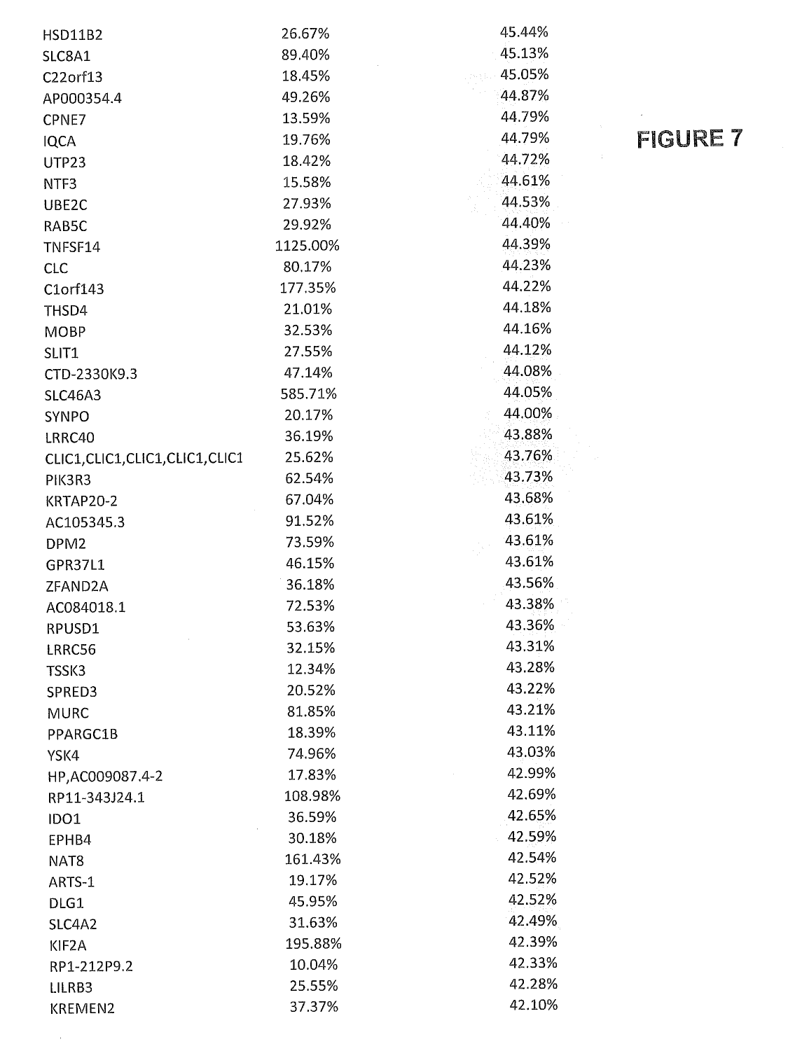

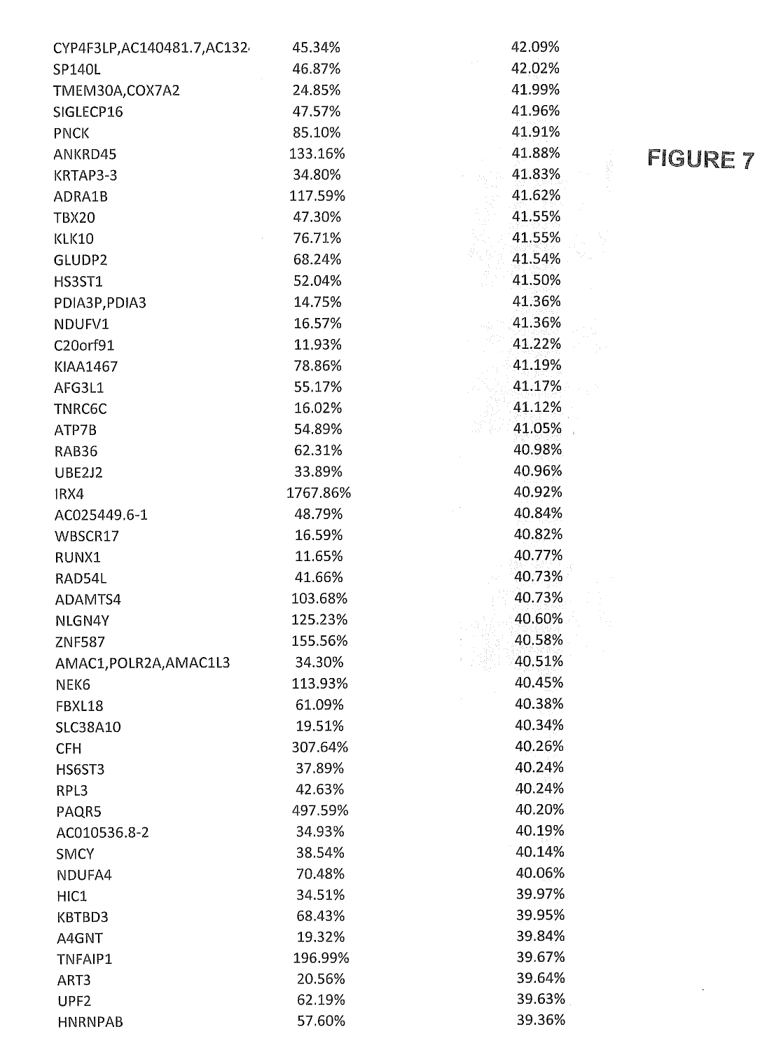

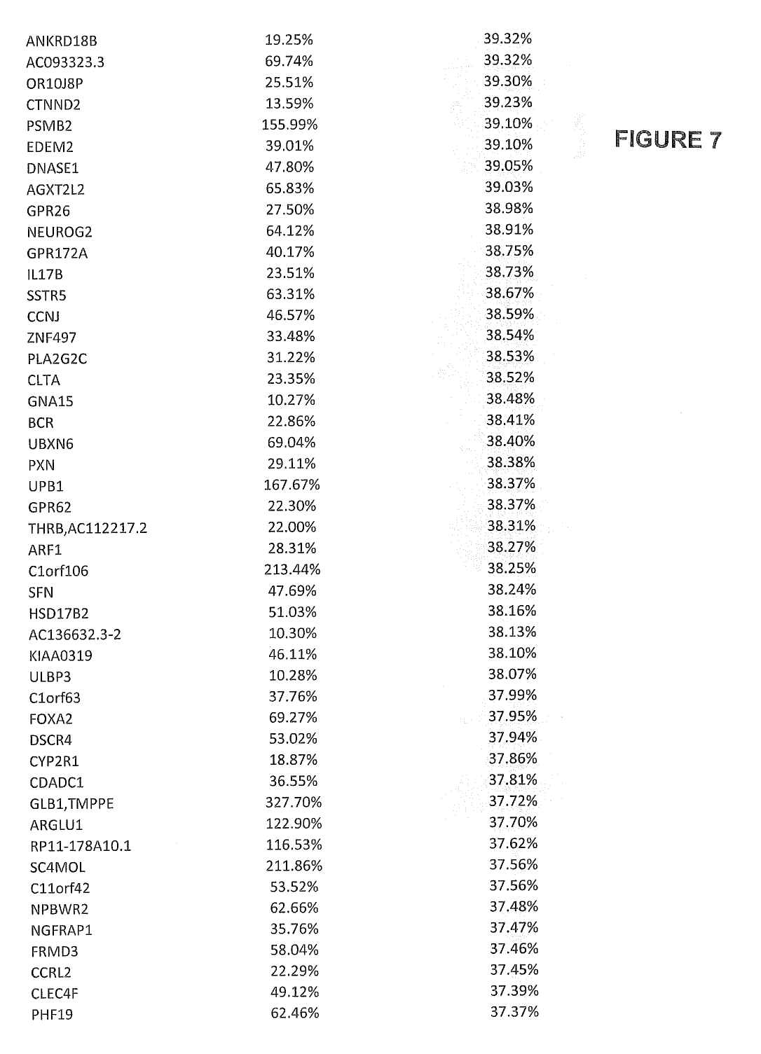

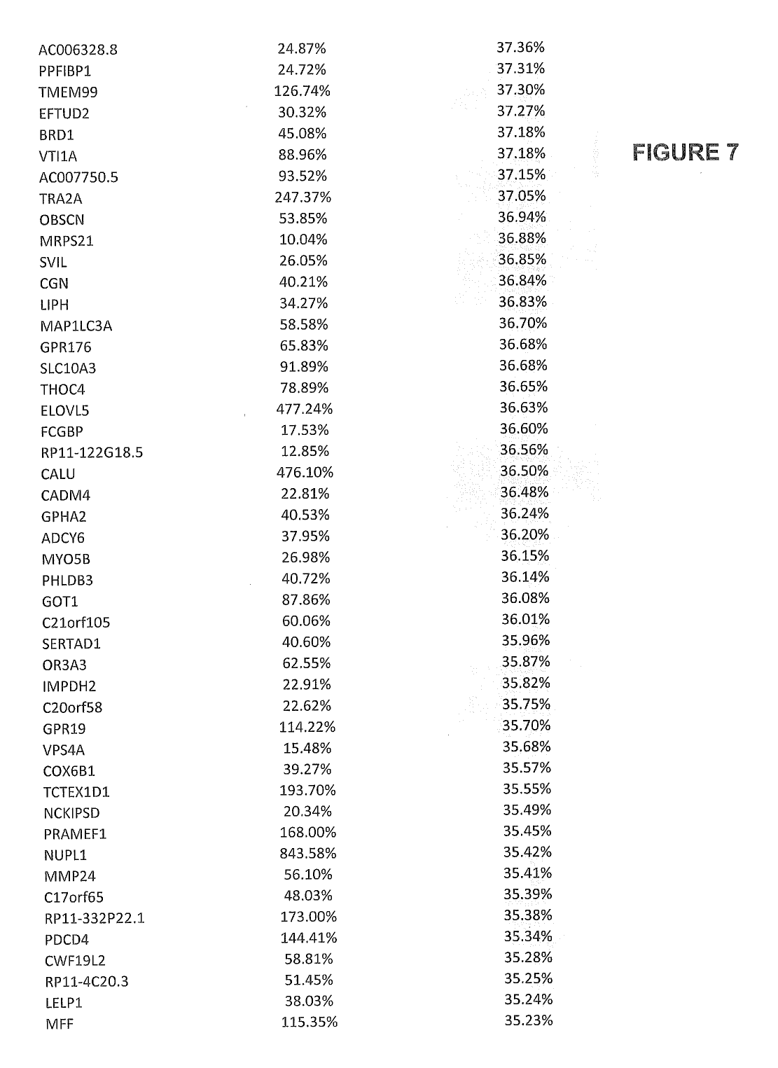

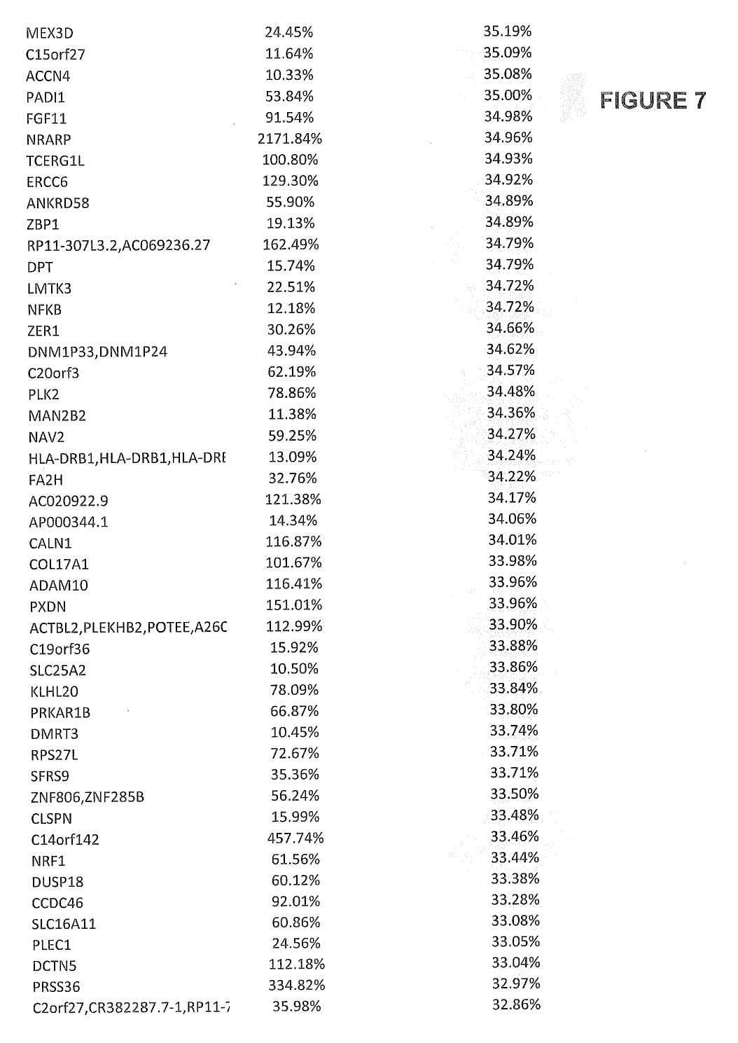

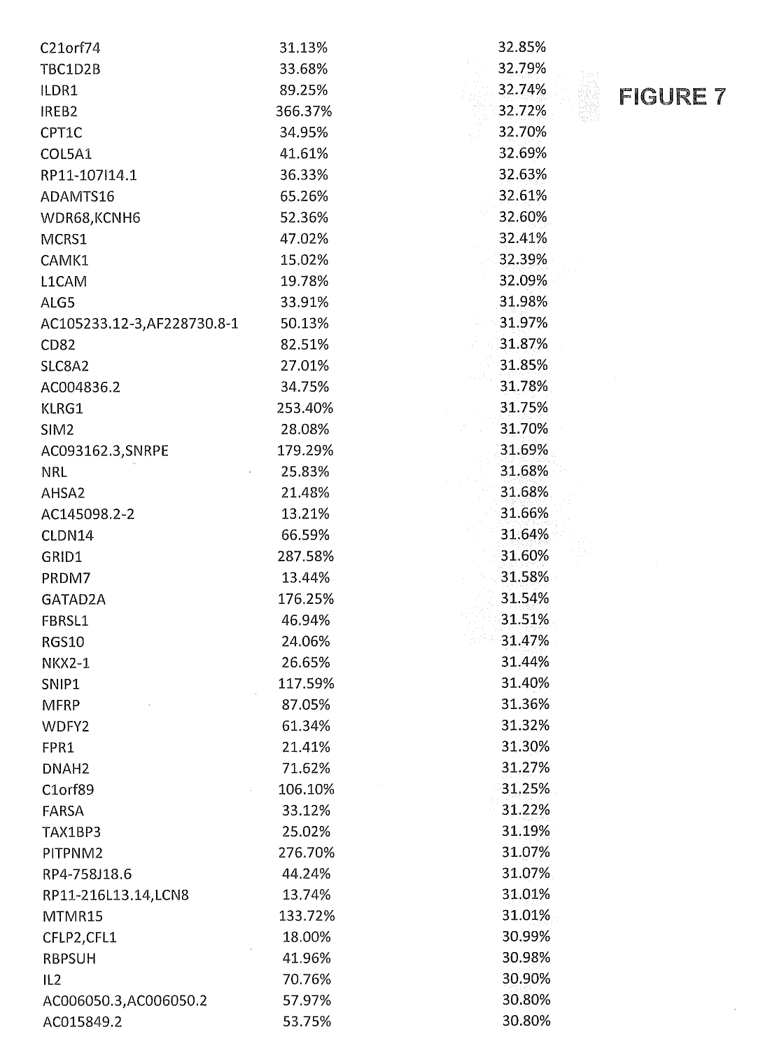

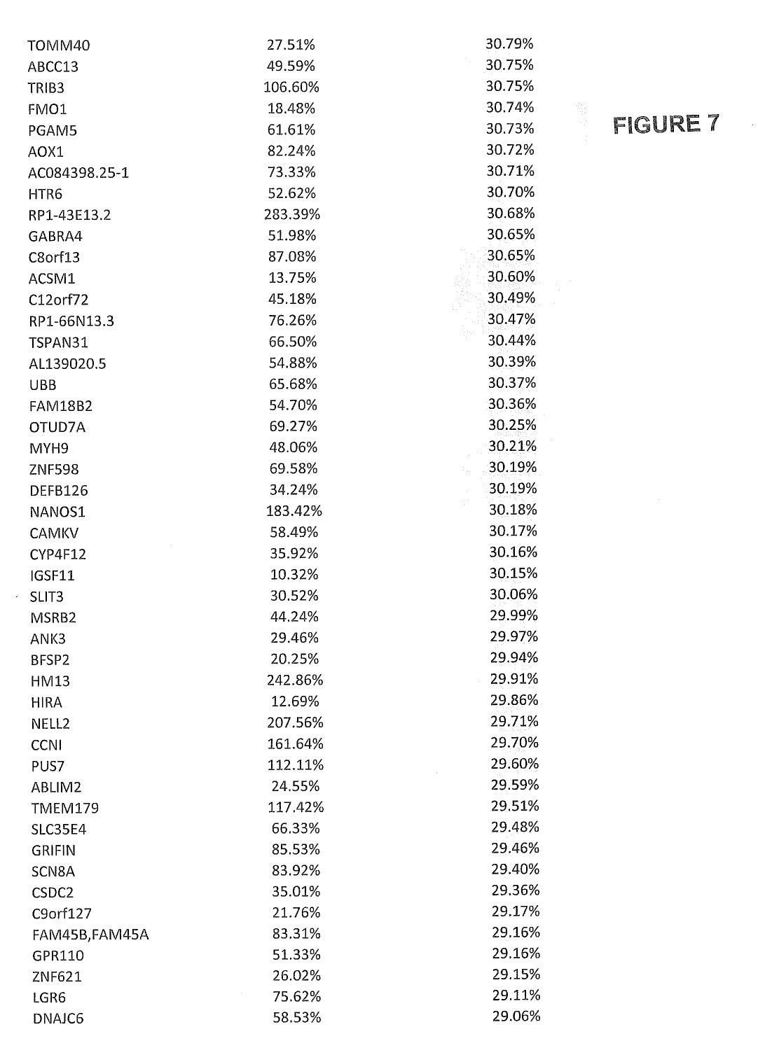

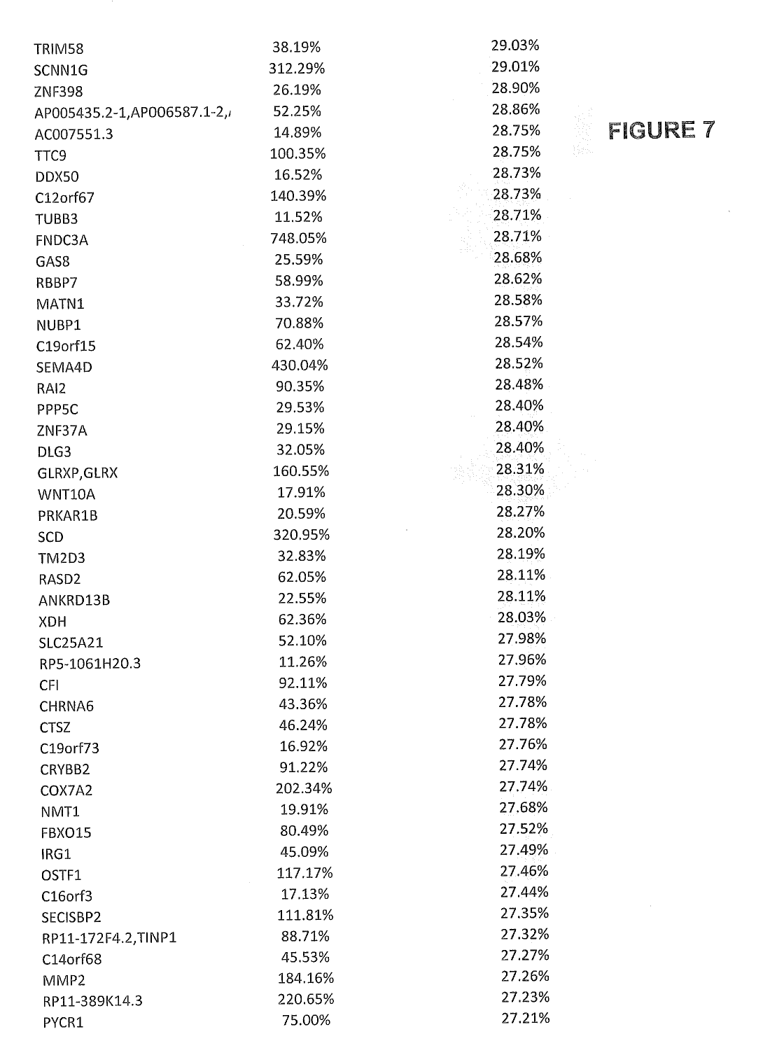

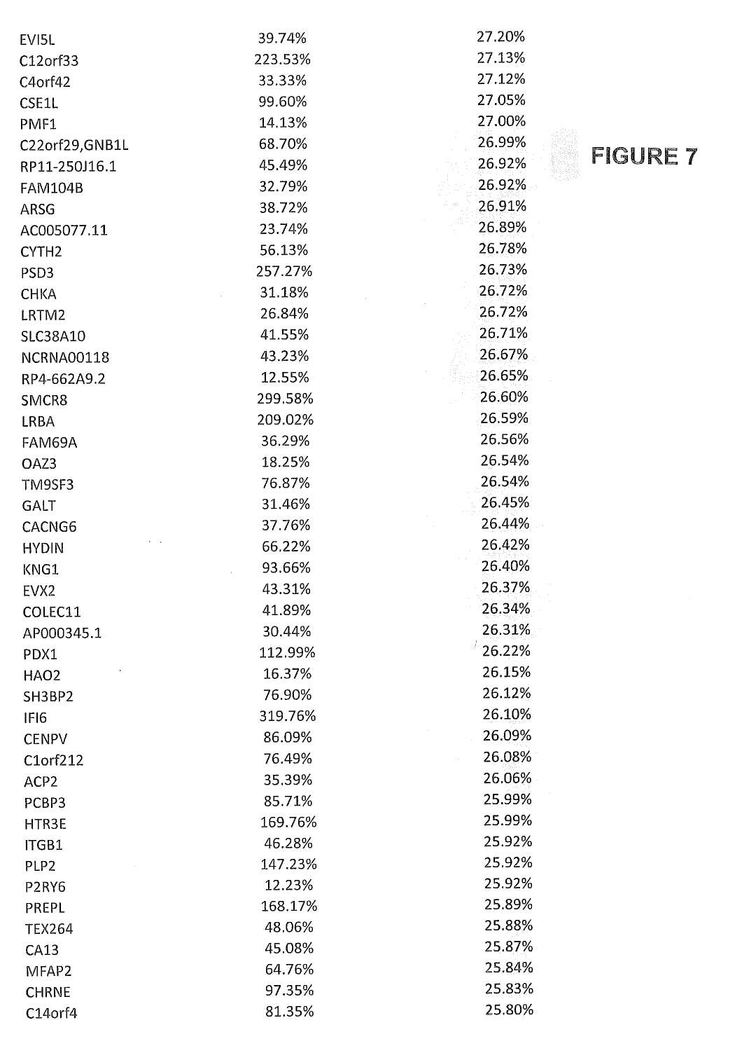

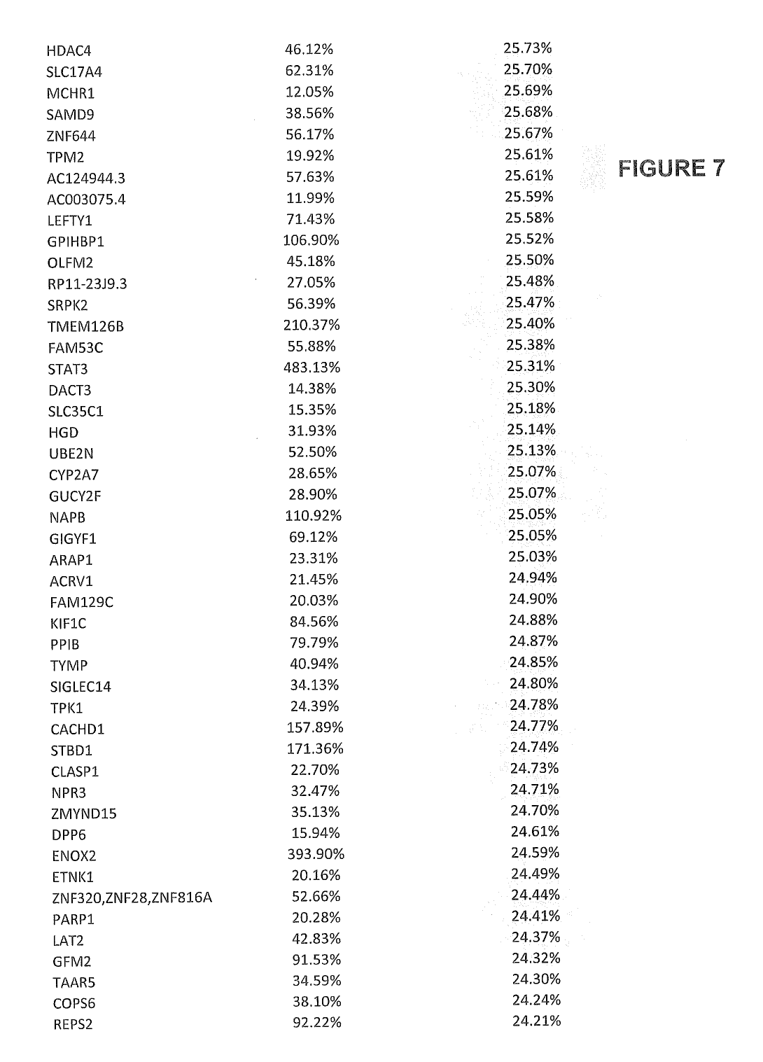

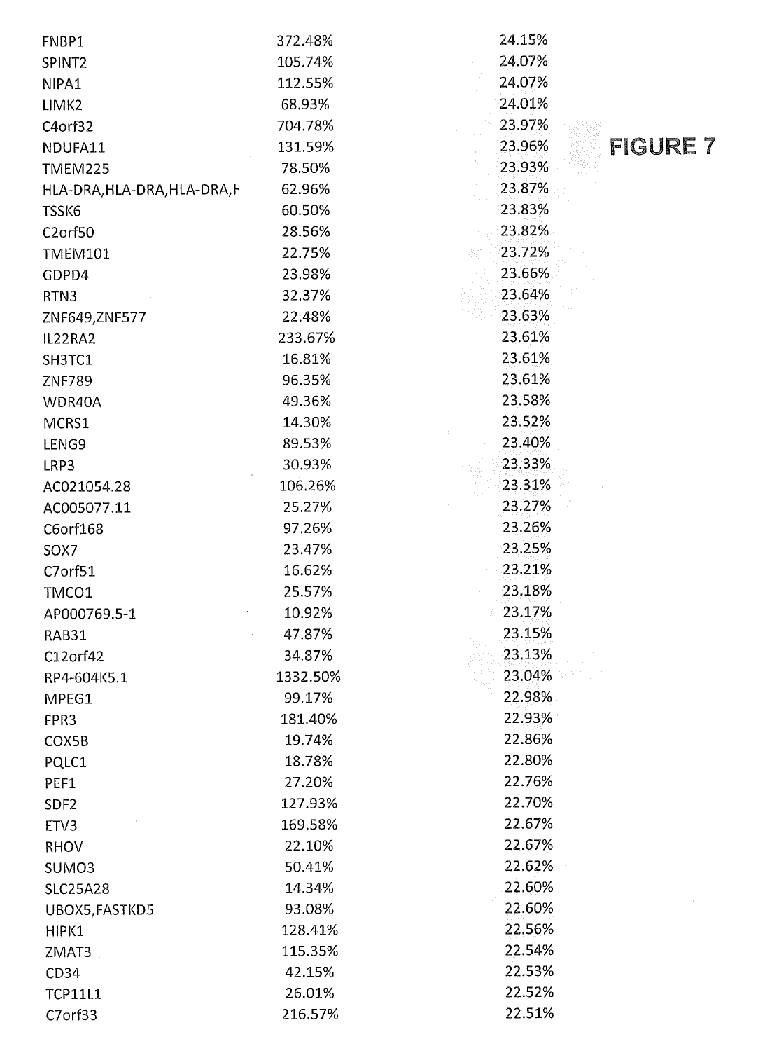

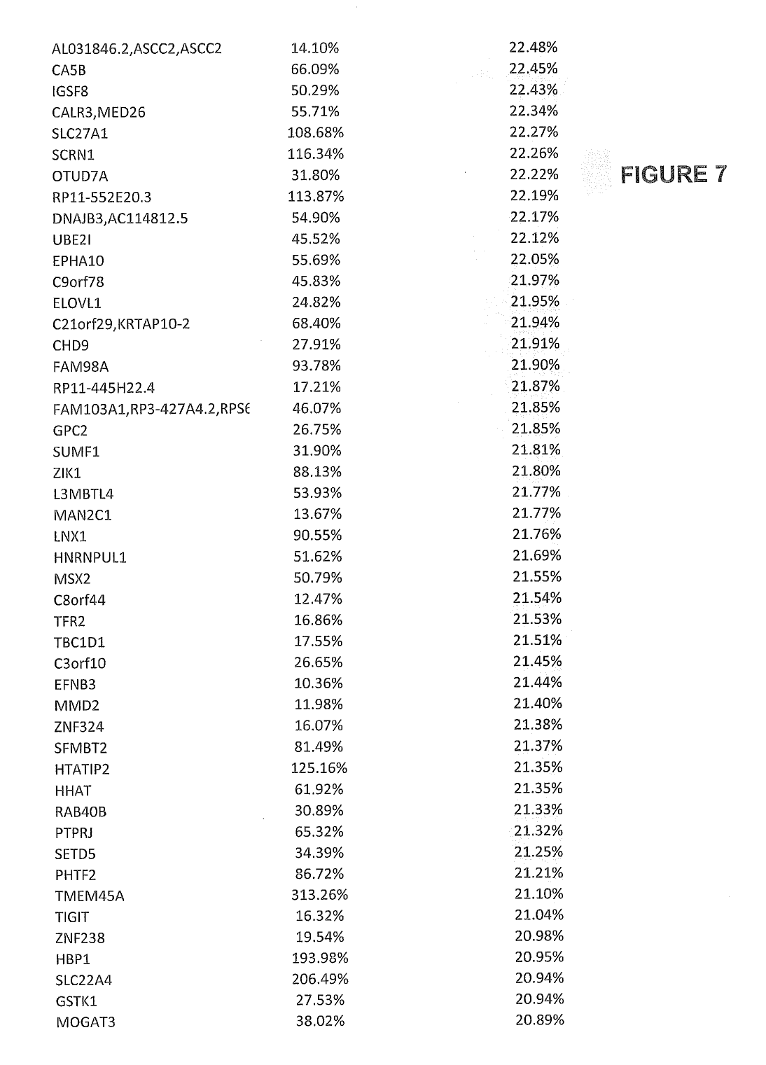

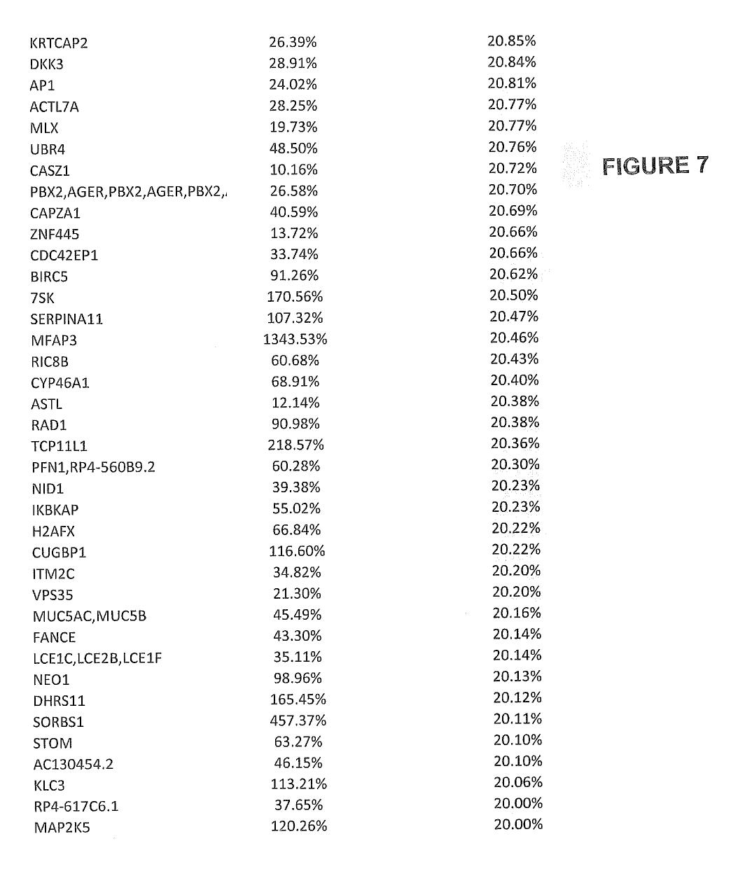

Downregulation observed at the gene level was verified at the protein level for several representative molecules using Western blotting or flow cytometry. A complete list of those genes downregulated or upregulated by each fullerene derivative (>20%) are shown in FIG. 7.

### Mouse Models of Anaphylaxis and Assessment of Toxicity

MC-dependent anaphylaxis and treatments are described in Ryan, J. J., Bateman, H. R., Stover, A., Gomez, G., Norton, S. K., Zhao, W., Schwartz, L. B., Lenk, R., and Kepley, C. L. J Immunol. 179, 665-672 (2007). Female C57BL/6J mice (Jackson Laboratory, Bar Harbor, Me.) aged 10-12 weeks were injected i.p. with 50 μg IgE-DNP. Two hours later mice were injected i.p. with TGA, Inos (100 ng/200 μl in PBS), or 200 μl PBS alone as a vehicle control. After 16 hours mice were challenged i.p. with 100 μg DNP-BSA (Sigma-Aldrich) in 100 μl PBS. Body temperature measurements were recorded with a digital rectal thermometer every 10 minutes for a total of 50 minutes. Peripheral blood was harvested by cardiac puncture 50 minutes after antigen challenge and serum histamine measurements determined by ELISA.

To examine potential toxicity, alanine aminotranferease (ALT) and aspartate aminotransferase (AST) were measured in serum. These enzymes leak out into the general circulation when the liver is injured. Mice were treated with or without 1000 μg/100 μl (50 times more than used for the in vivo study) of TGA or Inos by tail vein injection and i.p. After two and 14 days mice were sacrificed and blood, obtained by cardiac puncture. ALT and AST activity were measured as described in Booth, G., Newham, P., Barlow, R., Raines, S., Zheng, B., and Han, S. Autoimmunity 41, 512-521 (2008). Data is presented as an average of 4 (Untreated) or 4 (Treated) mice±Standard Deviation.

Fullerene Derivatives Reduce Degranulation and Cytokine Production from MC after Anti-FcεRI Activation

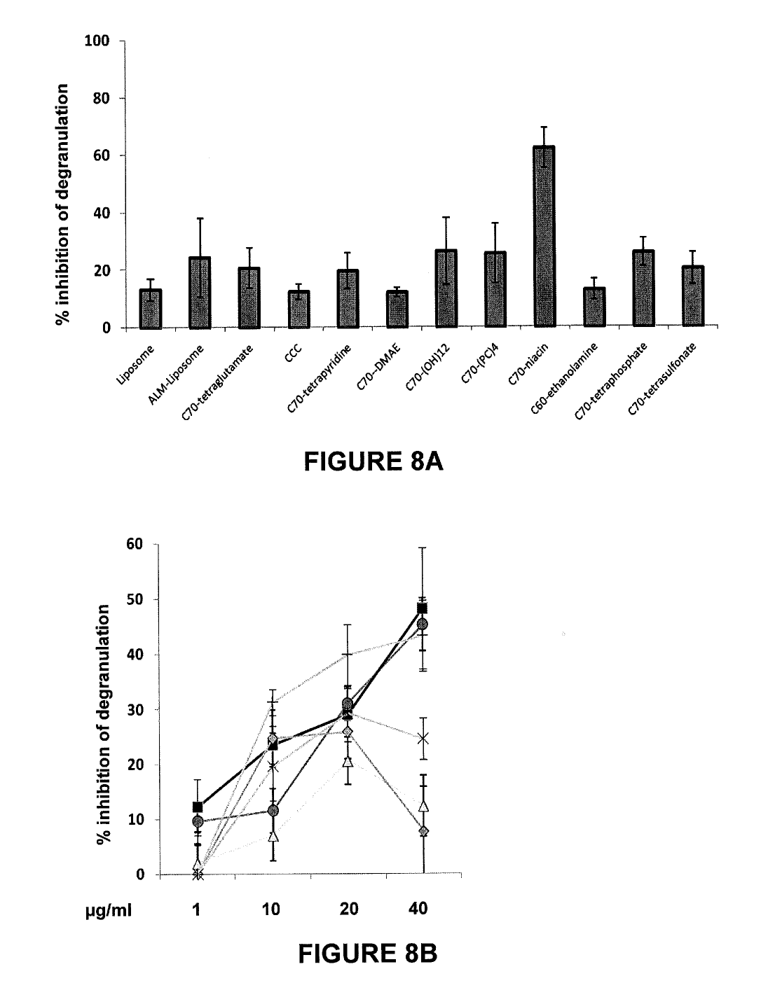

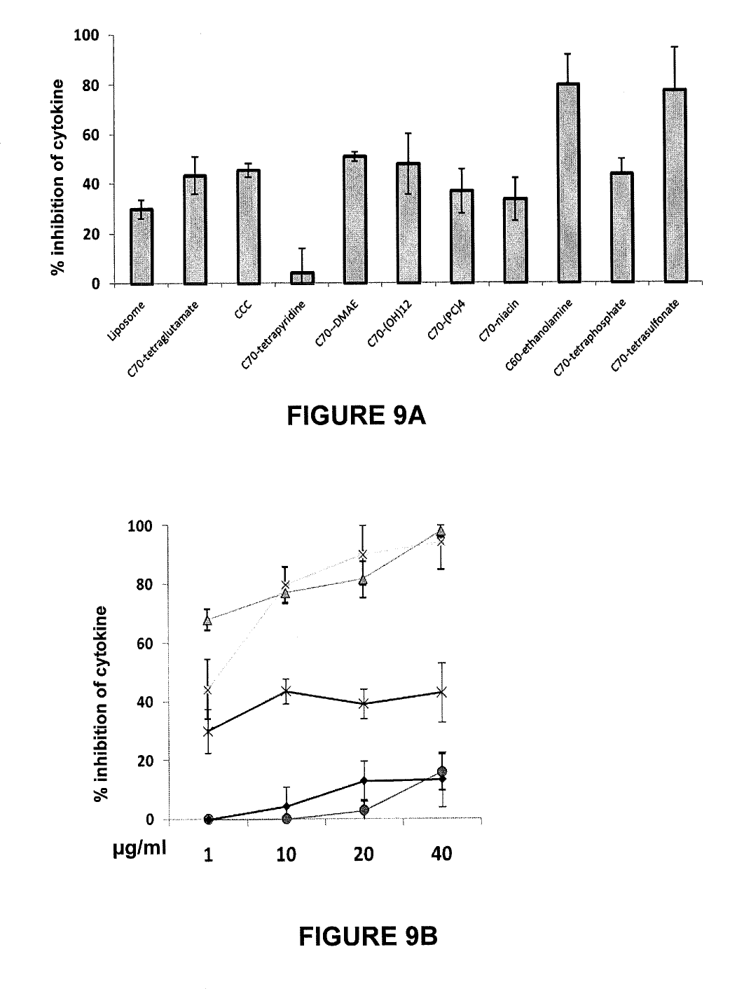

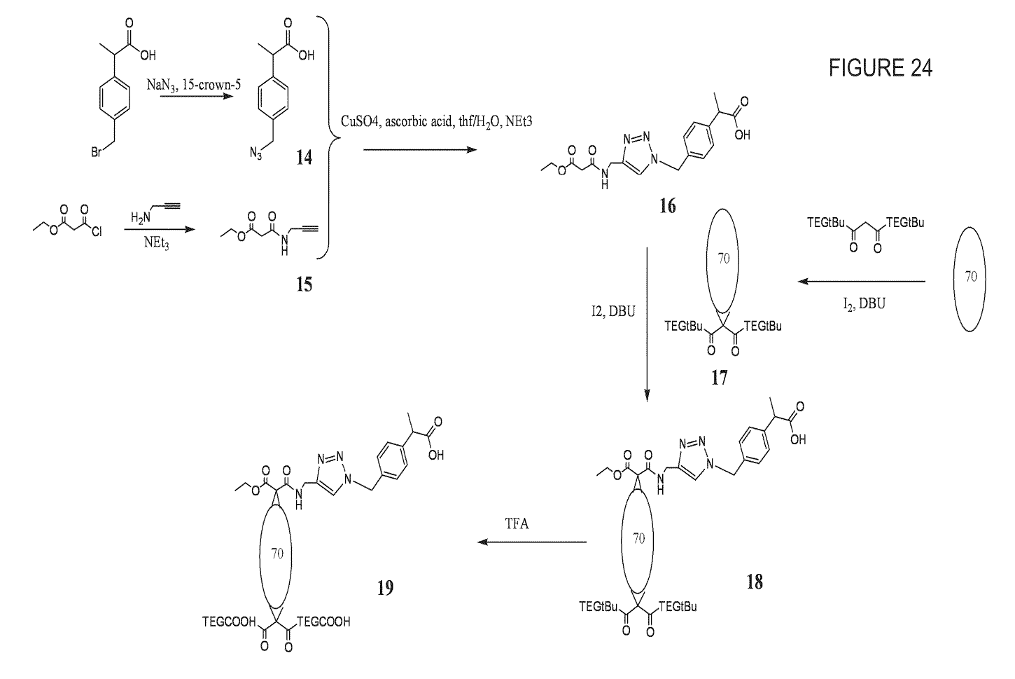

Cells were cultured with fixed concentrations of a fullerene derivative (10 μg/ml; FIGS. 8A and 9A) or different concentrations (FIGS. 8B and 9B) for 16 h, washed and stimulated for 30 min (FIGS. 8A and 8B) or 24 hours (FIGS. 9A and 9B) with optimal concentrations of αanti-FcεRI Abs (3B4; 1 μg/ml). Cells were centrifuged and β-hexosaminidase release and GMCSF levels determined by ELISA.

In FIGS. 8A and 9A, data shown are means±SE of triplicate samples that is representative of at least four separate experiments with separate MC cultures. All values shown demonstrated a significant (P<0.05) inhibition of at least >10% inhibition compared to non-treated samples. In FIG. 8B, C₇₀—(OH)₁₂ (black square), C₇₀-tetraphosphate (grey diamond), C₇₀-tetrapyridine (black X), C₇₀-niacin (grey circle), C₇₀—(PC)₄ (grey triangle), and CCC (black +). In FIG. 9B, C₇₀-tetrapyridine (black diamond), C₇₀-tetraphosphate (black *), C₇₀-tetrasulfonate (grey triangle), C₆₀-ethanolamine (black X), and CCC (grey circle). Fullerene derivatives with no effect (approximately 76% of those tested) are not shown.

Further, C₇₀—(PC)₄, C₇₀-tetrainositol and C₇₀-tetraglucolate (TGA) showed >10% inhibition of histamine release when screened at 5 μg/mL, compared to the relevant vehicle control (PBS or 10% DMSO in PBS) (Table 1). In addition, these fullerene derivatives showed significant inhibition of FcεRI-induced IL-13 release (optimal concentrations of anti-IgE was 15 ng/ml) (Table 2).

TABLE 1
Inhibition of Histamine Release

| | | | | Mean % |
|---|---|---|---|---|
| | Vehicle | | | Inhibition |
| | control + | FD + | P value | of |
| Fullerene | anti-IgE | anti-IgE | (n); | Histamine |
| Derivative | (mean ± SEM) | (mean ± SEM) | T-test | Release |
| C₇₀-(PC)4 | 46.2 ± 5.0 | 42.0 ± 4.7 | 0.098 (6) | 9.18 |
| C₇₀- | 50.8 ± 9.2 | 42.1 ± 9.7 | 0.007 (7) | 22.1 |
| Tetrainositol | | | | |
| C₇₀-TGA | 45.4 ± 6.7 | 39.4 ± 2.3 | 0.001 (9) | 15.8 |

TABLE 2
Inhibition of IL-13 Secretion

| | Vehicle | | | Mean % |
|---|---|---|---|---|
| | control + | FD + | | Inhibition |
| | anti-IgE | anti-IgE | P value | of |
| Fullerene | (mean ± SEM, | (mean ± SEM, | (n); | IL-13 |
| Derivative | ng/ml) | ng/ml) | T-test | Secretion |
| C₇₀-(PC)4 | 101.1 ± 20.3 | 37.6 ± 20.7 | 0.047 (3) | 62.7 |
| C₇₀- | 177.3 ± 61.0 | 6.0 ± 0.8 | 0.05 (3) | 90.9 |
| Tetrainositol | | | | |
| C₇₀-TGA | 197.4 ± 46.2 | 79.3 ± 20.9 | 0.029 (4) | 52.4 |

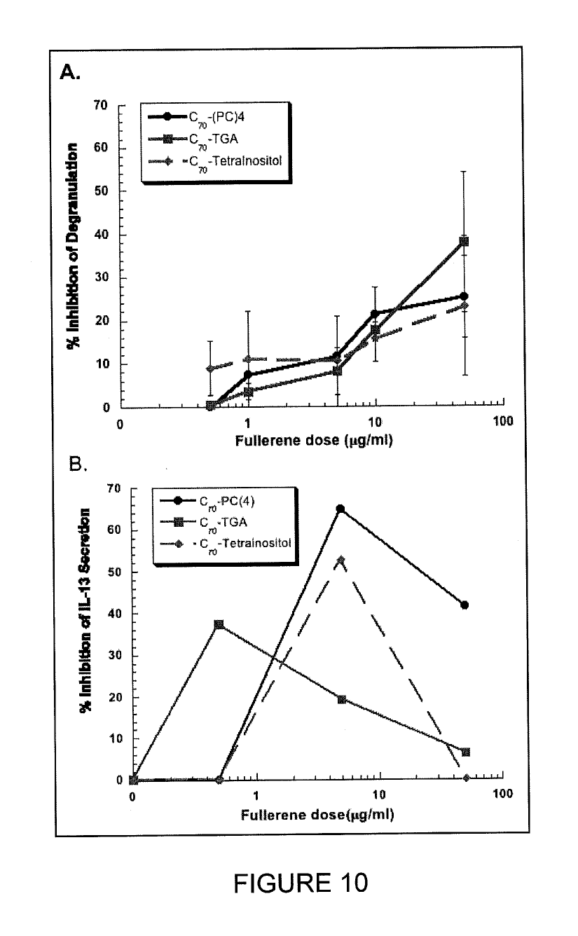

FIGS. 10A and 10B show concentration (dose)-dependent inhibition of histamine release and IL-13 release. As shown in FIG. 10A, the maximal inhibition of anti-IgE-induced histamine release was observed at the 50 mg/ml dose (C₇₀—(PC)₄=25.1±9.5%; C₇₀-tetrainositol=23±16.3%; and C₇₀-TGA=37.8±16.25%; mean±SEM, n=2-3). As shown in FIG. 10B, the basophils were more sensitive to inhibition of anti-IgE stimulated IL-13 secretion with maximal inhibition detected at 5 μg/ml for C₇₀—(PC)₄ (64.8%), 5 μg/ml for C₇₀-tetrainositol (52.5%); and 0.5 μg/ml for C₇₀-TGA (37.4%); mean, n=2).

Following Tables 3-6 show the results obtained with two fullerene derivatives: TGA and Inos. TGA was observed to be one of the most efficient inhibitors and significantly reduced both degranulation and cytokine production in MC and PBB. However, Inos significantly inhibited degranulation and cytokine production in PBB (Tables 5 and 6) but significantly inhibited only cytokine production in MC; degranulation was not affected in MC.

TABLE 3
Mean percent degranulation (±SD) in TGA treated and untreated MC
Mast cells

| | no XL | XL | TGA + XL [symbol] * | % inh | IC50 (μg/ml) |
|---|---|---|---|---|---|
| Exp 1 | 3.4 | 76.7 | 39.1 | 48.9 | |
| Exp 2 | 3.2 | 81.1 | 50.1 | 38.2 | |
| Exp 3 | 5.5 | 74.5 | 53.4 | 28.3 | |
| Exp 4 | 4 | 77.9 | 40.1 | 48.5 | |
| Exp 5 | 8.1 | 78.5 | 52.7 | 32.8 | |
| Average | 4.8 (±2.0) | 77.7 (±2.4) | 47.1 (±6.9) | 39.3 (±9.2) | 10.9 (±3.5) |

[symbol]

: TGA Treatment @ 10 μg/ml
* P value = 0.0001

Mean percent GMCSF-cytokine production (±SD) in TGA treated and untreated MC
Mast cells

| | no XL | XL | TGA + XL [symbol] * | % inh | IC50 (μg/ml) |
|---|---|---|---|---|---|
| Exp 1 | 50.4 | 211.4 | 69.5 | 67.8 | |
| Exp 2 | 135.4 | 893.8 | 121.9 | 84.9 | |
| Exp 3 | 91.6 | 309.5 | 104.5 | 66.2 | |
| Exp 4 | 65.4 | 271.4 | 74.1 | 72.7 | |
| Exp 5 | 105.4 | 741 | 43.4 | 95.2 | |
| Average | 89.6 (±33.4) | 485.4 (±309.8) | 82.7 (±30.8) | 77.4 (±12.4) | 3.3 (±0.6) |

[symbol]

: TGA Treatment @ 10 μg/ml
* P value = 0.02

TABLE 4
Mean percent degranulation (±SD) in Inos treated and untreated MC
Mast cells

| | no XL | XL | Inos + XL [symbol] ⁺ | % inh | IC50 (μg/ml) |
|---|---|---|---|---|---|
| Exp 1 | 3.4 | 76.7 | 78.1 | ⁻1.9 | |
| Exp 2 | 3.2 | 81.1 | 80.3 | 1 | |
| Exp 3 | 5.5 | 74.5 | 70.2 | 5.8 | |
| Exp 4 | 4 | 77.9 | 75.5 | 3.1 | |
| Exp 5 | 8.1 | 78.5 | 91.6 | ⁻16.8 | |
| Average | 4.8 (±2.0) | 77.7 (±2.4) | 79.1 (±7.9) | 3.3 (±2.4) | |

[symbol]

Inos Treatment @ 10 μg/ml
⁺P value = 0.358

Mean percent GMCSF-cytokine production (±SD) in Inos treated and untreated MC
Mast cells

| | no XL | XL | Inos + XLT [symbol] ⁺ | % inh | IC50 (μg/ml) |
|---|---|---|---|---|---|
| Exp 1 | 50.4 | 211.4 | 11.9 | 94.4 | |
| Exp 2 | 135.4 | 893.8 | 20.4 | 97.7 | |
| Exp 3 | 91.6 | 309.5 | 23.5 | 92.4 | |
| Exp 4 | 65.4 | 271.4 | 63.1 | 76.8 | |
| Exp 5 | 105.4 | 741 | 43.4 | 95.2 | |
| Average | 89.6 (±33.4) | 485.4 (±309.8) | 32.5 (±20.6) | 91.3 (±8.3) | 1.9 (±1.1) |

[symbol]

Inos Treatment @ 10 μg/ml
⁺P value = 0.006

TABLE 5
Mean percent degranulation (±SD) in TGA
treated and untreated PBB
Peripheral Blood Basophils

| | | | | | IC50 |
|---|---|---|---|---|---|
| | no XL | XL | TGA + XL [symbol] ¹ | % inh | (μg/ml) |
| Exp 1 | 7.3 | 35 | 31 | 11.4 | |
| Exp 2 | 22.2 | 37 | 34 | 8.1 | |
| Exp 3 | 16.8 | 36 | 24 | 33.3 | |
| Exp 4 | 8.1 | 43 | 40 | 6.9 | |
| Exp 5 | 9.5 | 21 | 17 | 19 | |
| Exp 6 | 17.8 | 62.5 | 51 | 18.4 | |
| Exp 7 | 9.2 | 64 | 63 | 1.56 | |
| Exp 8 | 11 | 83 | 78 | 6 | |
| Exp 9 | 19.2 | 27.5 | 17 | 38.1 | |
| Average | 13.5 | 45.4 | 39.4 | 15.8 | 9.9 |
| | (±5.5) | (±20.1) | (±7.0) | (±4.2) | (±2.9) |

[symbol]

TGA Treatment @ 5 μg/ml
¹P value = 0.001
Mean percent IL-13-cytokine production(±SD) in TGA
treated and untreated PBB
Peripheral Blood Basophils

| | | | | | IC50 |
|---|---|---|---|---|---|
| | no XL | XL | TGA + XL [symbol] ¹ | % inh | (μg/ml) |
| Exp 1 | 42.8 | 25.2 | 119.5 | 52.6 | |
| Exp 2 | 12.6 | 297.5 | 35 | 88.2 | |
| Exp 3 | 16.4 | 102.5 | 52.5 | 48.8 | |
| Exp 4 | 39.4 | 137.5 | 110 | 20 | |
| Average | 27.8 | 197.4 | 79.3 | 52.4 | 10.6 |
| | (±92.4) | (±92.4) | (±41.8) | (±28.0) | (±3.4) |

[symbol]

TGA Treatment @ 5 μg/ml
¹P value = 0.029

TABLE 6
Mean percent degranulation (±SD) in Inos
treated and untreated PBB
Peripheral Blood Basophils

| | | | | | IC50 |
|---|---|---|---|---|---|
| | no XL | XL | Inos + XL [symbol] * | % inh | (μg/ml) |
| Exp 1 | 7.3 | 35 | 23 | 34.2 | |
| Exp 2 | 16.8 | 36 | 21 | 41.6 | |
| Exp 3 | 12.6 | 41 | 36 | 12.1 | |
| Exp 4 | 10.4 | 19 | 11 | 42.1 | |
| Exp 5 | 12.9 | 62.5 | 60 | 4 | |
| Exp 6 | 12.5 | 86 | 68 | 20.9 | |
| Exp 7 | 11.4 | 76 | 76 | 0 | |
| Average | 12 | 50.8 | 42.1 | 22.1 | 17.8 |
| | (±2.9) | (±24.5) | (±25.7) | (±17.5) | (±3.2) |

[symbol]

: Inos Treatment @ 5 μg/ml
* P value = 0.007
Mean percent IL-13-cytokine production (±SD) in Inos
treated and untreated PBB
Peripheral Blood Basophils

| | | L | | | IC50 |
|---|---|---|---|---|---|
| | no XL | X | Inos + XL [symbol] * | % inh | (μg/ml) |
| Exp 1 | 42.8 | 252 | 7 | 97.2 | |
| Exp 2 | 16.4 | 102.5 | 5 | 95.1 | |
| Exp 3 | 1.2 | 51.8 | 10.1 | 80.5 | |
| Average | 29.6 | 177.3 | 6.0 | 90.9 | 2.6 |
| | (±18.7) | (±105.7) | (±1.4) | (±9.0) | (±1.2) |

[symbol]

: Inos Treatment @ 5 μg/ml
* P value = 0.05

IC50 values was calculated by setting the fullerene derivative dose that resulted in maximum inhibition to 100% and then extrapolating the dose at which 50% inhibition was seen. The majority of the compounds tested to date had no significant effect on FcεRI mediator release. These data demonstrate selective inhibition of FcεRI mediator release from primary human MC/PBB using nano-engineered fullerene derivatives which depends on the moieties added to the carbon cage.

Unlike TGA, Inos does not interfere with degranulation in MC but is highly effective at blocking cytokine production in MC, while it does inhibit degranulation in PBB. This suggests that the Inos interacts and/or indirectly inhibits a signaling molecule found in human MC FcεRI signaling and not in PBB.

Mechanistically, the variations in how Inos and TGA differentially inhibit intercellular FcεRI responses between MC and PBB may be explained in how they affect Lyn. In mouse Lyn−/− basophils, FcεRI-mediated degranulation is inhibited. Charles, N., Watford, W. T., Ramos, H. L., Hellman, L., Oettgen, H. C., Gomez, G., Ryan, J. J., O'Shea, J. J., and Rivera, J. Immunity. 30, 533-543 (2009). However, Lyn−/− BMMCs can have the opposite phenotype with degranulation being upregulated or inhibited depending on the Lyn/Fyn ratio. Parravicini, V., Gadina, M., Kovarova, M., Odom, S., Gonzalez-Espinosa, C., Furumoto, Y., Saitoh, S., Samelson, L. E., O'Shea, J. J., and Rivera, J. Nat. Immunol. 3, 741-748 (2002); and Yamashita, Y., Charles, N., Furumoto, Y., Odom, S., Yamashita, T., Gilfillan, A. M., Constant, S., Bower, M. A., Ryan, J. J., and Rivera, J. J. Immunol. 179, 740-743 (2007). In RBL cells degranulation can be inhibited while TNF-α secretion is unaffected by overexpressing Lyn. Vonakis, B. M., Gibbons, S. P., Jr., Rotte, M. J., Brothers, E. A., Kim, S. C., Chichester, K., and MacDonald, S. M. J. Immunol. 175, 4543-4554 (2005).

There is also precedence demonstrating that intracellular FcεRI-signaling pathways diverge subsequent to activation in MC and PBB. Concentrations of FcεRI cross-linking agents leading to optimal cytokine production are consistently lower than concentrations needed for optimal degranulation. MacGlashan, D., Jr. Immunology Series 57, 273-299 (1992). The release of pre-formed mediators through FcεRI-mediated degranulation follows the activation of PKC and calcium mobilization, cytokine and chemokine production requires activation of MAP kinases p38 and JNK, whereas lipid mediator production follows the activation of ERK1/2 pathway. Gilfillan, A. M. and Tkaczyk, C. Nat. Rev. Immunol. 6, 218-230(2006).

A central control point that possibly mediates FcεRI-signals leading to cytokine production and mediator release occurs at LAT, the phospho-activation of this signaling molecule was affected significantly (see below). Rivera, J. and Gilfillan, A. M. J. Allergy Clin. Immunol. 117, 1214-1225 (2006).

In mice, tumor necrosis factor-associated factor 6 (TRAF6) is specifically required for cytokine generating FcεRI-signals of NF-κB, p38 MAP kinase and JNK yet is not required for proximal signaling and subsequent degranulation. Yang, Y. J., Chen, W., Carrigan, S. O., Chen, W. M., Roth, K., Akiyama, T., Inoue, J., Marshall, J. S., Berman, J. N., and Lin, T. J. J. Biol. Chem. 283, 32110-32118 (2008).

### Mechanisms of MC-FcεRI Inhibition

To understand how TGA and Inos differentially influence FcεRI-dependent mediator release, those events that occur immediately (within 30 minutes) upon FcεRI activation were first examined. The activation of MC and PBB FcεRI leading to degranulation is calcium dependent and induces elevated cellular levels of ROS. Swindle, E. J. and Metcalfe, D. D. Immunol. Rev. 217:186-205., 186-205 (2007). It is hypothesized that the underlying mechanism of inhibition involved the blocking of FcεRI-mediated calcium and ROS responses based on previous studies with mixed isomer fullerene derivatives. Ryan, J. J., Bateman, H. R., Stover, A., Gomez, G., Norton, S. K., Zhao, W., Schwartz, L. B., Lenk, R., and Kepley, C. L. J Immunol. 179, 665-672 (2007).

The responses of MC treated with TGA or Inos were compared.

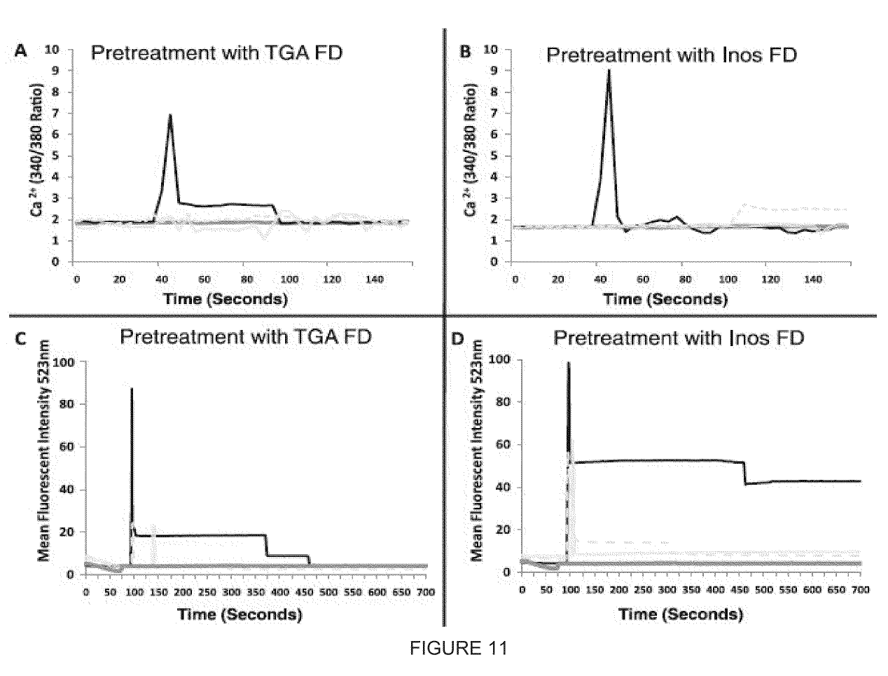

Cells were incubated with or without fullerene derivative (10 μg/mL) overnight. The next day cells were challenged with anti-FcεRI (3B4; 1 μg/mL) and calcium stores release was determined by the 340/380 nm ratio (FIGS. 11A and 11B) and ROS measured by DCF detection at 523 nm (FIGS. 11C and 11D). FIGS. 11A and 11C contain cells incubated with the TGA fullerene derivative, and FIGS. 11B and 11D contain cells incubated with the Inos fullerene derivative (10 μg/mL). The dark gray squares are the unchallenged negative control, dark thick solid black line is the anti-FcεRI challenged positive control cells, and the two light gray lines (hashed and double line) are the anti-FcεRI challenged cells pretreated with respective fullerene derivative. Results are representative of at least three separate experiments.

The increase in FcεRI-induced intracellular calcium stores release (FIG. 11A) and ROS (FIG. 11B) upon FcεRI cross-linking was inhibited with TGA. However, Inos pre-incubation did not affect calcium (FIG. 11C) or ROS (FIG. 11D) levels. Gene microarray data further suggests that TGA and Inos differentially influence FcεRI-associated signaling molecules involved in calcium stores release and oxidative stress. Thus, TGA and Inos differentially affect ROS and calcium responses induced by FcεRI activation.

### Fullerene Derivatives Block Early FcεRI-Activated Signaling Molecules

To further investigate the early signaling events in FcεRI-dependent mediator release that are affected by TGA or Inos, Western blotting analysis using phospho-specific antibodies were performed. The phosphorylation of signaling intermediates is an important early step in FcεRI-induced mediator release. Gilfillan, A. M. and Rivera, J. Immunol. Rev. 228, 149-169 (2009).

MC were pretreated with Inos or TGA (10 μg/mL) overnight at 37° C./6% CO₂. The next day washed cells were activated with or without anti-FcεRI (1 μg/mL) for the indicated times and lysed using protocols described above. Two separate 10% Tris-Glycine gels were used for sodium dodecyl sulfate polyacrylamide gel electrophoresis (SDS-PAGE) and Western blotting performed with the indicated phosphorylation-specific (i.e., phosph-Lyn) antibodies. To ensure equal loading, an antiactin antibody was used in parallel. The intensity of each band was detected and quantified using an Odyssey imaging system. Band intensities are presented as a ratio compared to antiactin band intensities probed in the same lane and presented for each time point in the colored graphs on the right side. Results are representative of three separate experiments. The two gels were run side-by-side, so that each time point could be examined, and are shown next to each other.

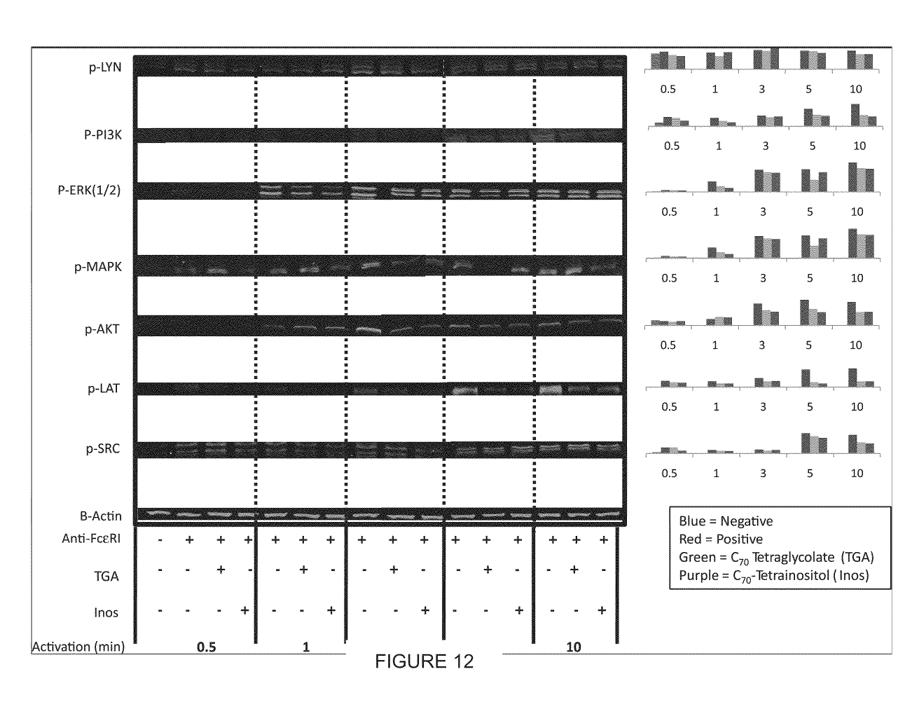

As seen in FIG. 12, several signaling intermediates that were phosphorylated by FcεRI activation were inhibited by pre-incubation with both fullerene derivatives including phosphorylation of extracellular signal regulated kinases 1/2 (ERK1/2), p38-mitogen-activated protein kinase (p38 MAPK), linker of activated T cells (LAT), AKT, phosphoinositide 3-kinase (PI3-K), and SRC. For example, fullerene derivative inhibition of MC signaling was time dependent as pre-incubation with TGA inhibited AKT phosphorylation 10%, 36%, and 43% at 3, 5 and, 10 minutes while Inos inhibited AKT phosphorylation 18%, 48%, and 42% at 3, 5 and, 10 minutes. Both fullerene derivatives dramatically reduced LAT phosphorylation at later time points with 36%, 72%, and 69% for TGA and 33%, 79%, and 70% for Inos at 3, 5 and, 10 minutes, respectively. PI3-kinase, a kinase strongly associated with calcium flux, was dramatically inhibited by both compounds with TGA reducing phosphorylation by 15%, 35%, and 50%, while Inos reduced phosphorylation by 8%, 40%, and 56% at 3, 5, and 10 minutes, respectively. There were also variations in the phosphorylation of other signaling intermediates examined at several time points. Little change was observed with a phospho-Lyn Ab to the negative regulatory tyrosine; a kinase with positive and negative signaling roles in MC. Rivera, J., Fierro, N. A., Olivera, A., and Suzuki, R. Adv. Immunol. 98, 85-120 (2008). Thus, molecules previously implicated in the release of calcium stores and ROS production in response to FcεRI aggregation were inhibited by certain fullerene derivatives. However, differences in how TGA and Inos inhibit FcεRI mediator release were not due to differential phosphorylation of the signaling molecules examined.

It was shown for the first time that fullerene derivatives can inhibit the phosphorylation of signaling intermediates involved with calcium and ROS generation. The Inos and TGA both reduced the phosphorylation of several intermediates; especially LAT and PI3-K. These results are consistent with other findings showing that LAT is critical for calcium mobilization and MC from LAT-deficient mice have inhibited FcεRI-mediated degranulation. Gilfillan, A. M. and Tkaczyk, C. Nat. Rev. Immunol. 6, 218-230 (2006). Similarly, PI3-K is critical for functional responses in MC as the PI3-K inhibitors wortmannin and LY294002 inhibit antigen-induced calcium mobilization, degranulation and cytokine production by murine and human MC. Gilfillan, A. M. and Rivera, J. Immunol. Rev. 228, 149-169 (2009). However, the activation of these signaling molecules is dependent on prior activation of Lyn. In this study, the early (10 minute) phosphorylation of Lyn was not influenced by either fullerene derivative.

### Gene Microarray Analysis of TGA and Inos-Treated MC

Given that optimal inhibition of MC or PBB secretion was observed after overnight pre-incubation with fullerene derivative, it was hypothesized that a transcriptional mechanism of action was involved.

Microarray analysis was used to obtain a broad overview of those FcεRI-associated signaling molecules influenced by TGA and Inos pre-incubation following FcεRI activation at later (2 hours) times. Of the approximately 31,000 genes examined the level of expression of 1,771 increased at least 10% in MCs following cross linking of FcεRI. In cells pretreated with TGA 2,603 of those were decreased at least 20% compared to non TGA treated cells and 515 demonstrated greater than 50% inhibition.

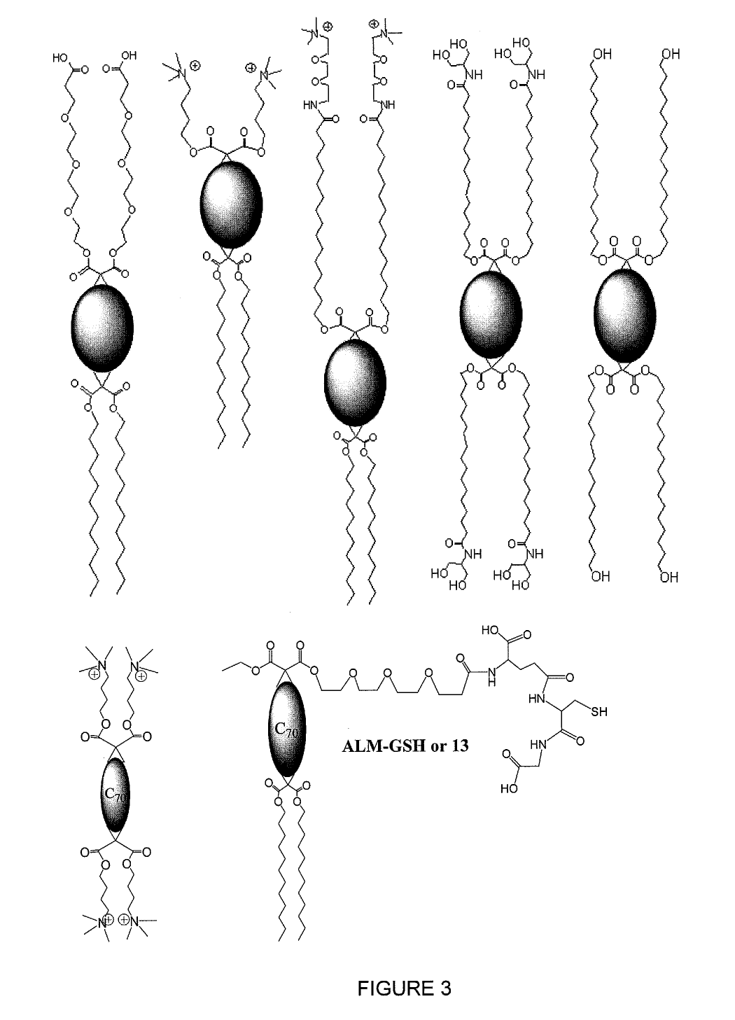

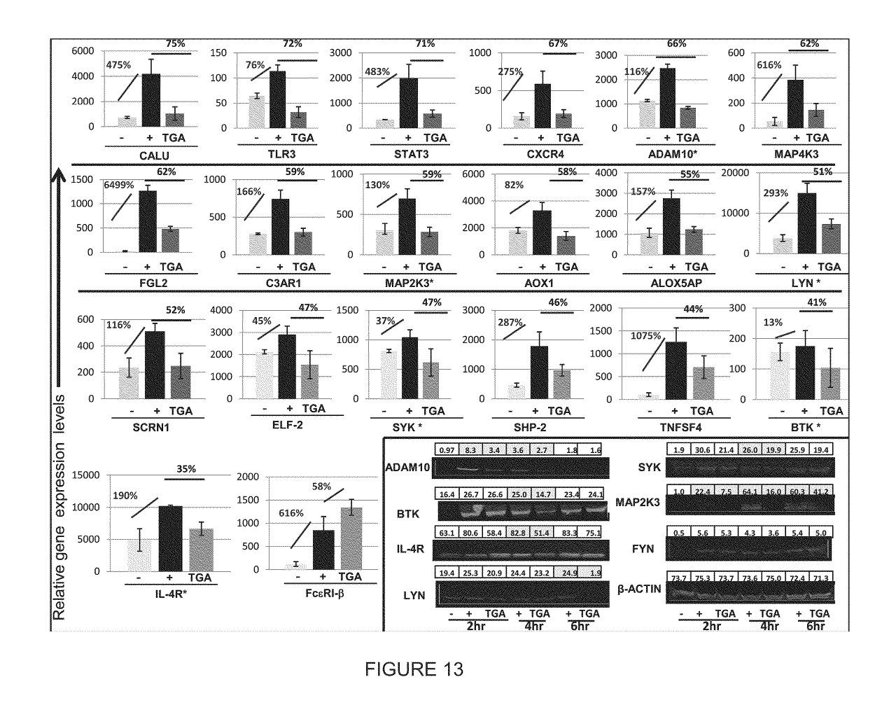

FIG. 13A represents several of those genes that were upregulated after 2 hours of FcεRI activation and significantly inhibited by TGA pre-incubation. The gene microarray data was verified at the protein level using Western blotting. For example, the gene for the tyrosine kinase Lyn was upregulated 293% upon FcεRI stimulation. However, gene expression levels were reduced by 51% at 2 hours which resulted in a 92% inhibition at the protein level observed at 6 hours (FIG. 13). E74-like factor 2 (ELF-2), which transcriptionally regulates Lyn, was also downregulated 47% suggesting Lyn is controlled at the transcription/translation level and protein level after two hours of FcεRI aggregation but not after 10 minutes of activation. Conversely, Fyn, which was not inhibited at the genetic level (not shown) had no change in protein levels when pre-incubated with TGA (FIG. 3-insert). Several other molecules including ADAM10, MAP2K, BTK, and Syk, were inhibited at the gene level which resulted in lower protein levels. The ability to down regulate multiple components of a signaling pathway is novel and may provide a platform for engineering potent anti-inflammatory compounds. These results indicate the ability to block both the activation as well as the expression of distinct signaling molecules within the FcεRI pathway.

There was a dramatic difference in the FcεRI genes affected by Inos compared to TGA. In cells preincubated with this fullerene derivative, only 741 genes were decreased at least 20% compared to non fullerene derivative treated cells and 92 demonstrated greater than 50% inhibition. Instead, there was a dramatic increase in expression of genes associated with inhibitory signaling.

The Inos fullerene derivative had 6 times more (1847) genes upregulated compare to TGA (FIG. 7).

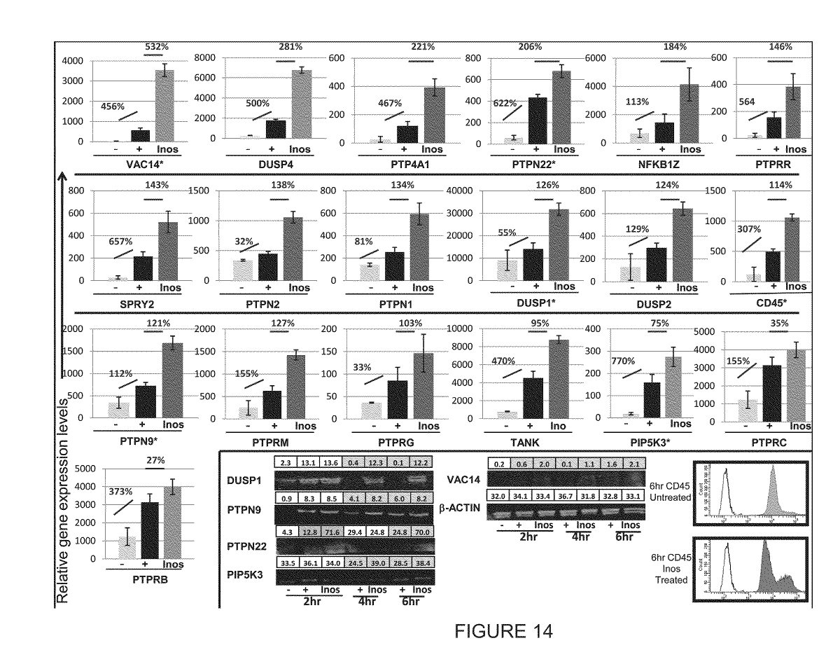

MC were treated and analyzed as in FIG. 13. Given the large size of CD45 (220 kDa) and difficulty in gel-to-membrane transfer FACS analysis was used to monitor upregulation: mouse IgG Isotype control (black line), untreated FITC labeled CD45 (gray filled), and Inos treated Fluorescein isothiocyanate (FITC) labeled CD45 (red filled) (FIG. 14).

Unlike with TGA, Inos induced the upregulation of over 20 protein tyrosine phosphatases (PTP) and dual specificity phosphatases (DUSP). The upregulation of several of these inhibitory molecules is shown in FIG. 14. For example, DUSP1 was upregulated 126% at the gene level at 2 hours and 99% at 6 hours at the protein level (FIG. 14-insert). Therefore, the inhibition of cytokine production by Inos (opposed to TGA) was due in part to increasing protein expression of PTP. Since TGA and Inos may be differentially affecting two groups of enzymes (kinases vs. PTP), the fullerene derivative may be recognizing a common regulatory mechanism or motif in the signaling molecule.

These results revealed that TGA profoundly reduced the FcεRI-induced activation of over 1000 genes which was selectively verified at the protein level. Several important discoveries were made from these experiments.

First, many upregulated genes were identified that were previously not associated with MC/PBB FcεRI signaling. These include tank binding kinase 1 (TBK-1) involved in mediating NFκB activation, Poly [ADP-ribose] polymerase 1 (PARP1) previously shown to be a target for the development of new therapeutic strategies in the treatment of lung disorders such as asthma, calumenin (CALU) which is a calcium-binding protein localized in the endoplasmic reticulum (ER) involved in protein folding and sorting, a disintegrin and metalloproteinase (ADAM10), SOX9, tumor necrosis factor alpha-induced protein 1 (TNFAIP1) an immediate-early response gene of endothelium induced by TNF-α, and SAMD9 involved in the regulation of TNF-α signaling. Pomerantz, J. L. and Baltimore, D. EMBO J. 18, 6694-6704 (1999); Boulares, A. H., Zoltoski, A. J., Sherif, Z. A., Jolly, P., Massaro, D., and Smulson, M. E. Am. J Respir. Cell Mol. Biol. 28, 322-329 (2003); Sarma, V., Wolf, F. W., Marks, R. M., Shows, T. B., and Dixit, V. M. J. Immunol. 148, 3302-3312 (1992); Topaz, 0., Indelman, M., Chefetz, I., Geiger, D., Metzker, A., Altschuler, Y., Choder, M., Bercovich, D., Uitto, J., Bergman, R., Richard, G., and Sprecher, E. Am. J. Hum. Genet. 79, 759-764 (2006); and Chefetz, I., Ben, A. D., Browning, S., Skorecki, K., Adir, N., Thomas, M. G., Kogleck, L., Topaz, O., Indelman, M., Uitto, J., Richard, G., Bradman, N., and Sprecher, E. J. Invest Dermatol. 128, 1423-1429 (2008).

Second, these were the first data to demonstrate that fullerene derivatives can influence gene expression. Given that current dogma suggest their biological activity depends solely on their anti-oxidant properties these results suggest fullerene derivative effects are not entirely due to ROS scavenging capabilities.

Third, it proves that fullerenes as a class cannot be considered to behave the same in vitro, in situ, and in vivo. Two C₇₀-based derivatives with very similar molecular weights had practically no common effects in the microarray studies. This suggests that the biological effects of fullerene derivatives critically depends on the side chains added to the core carbon cage.

It was observed that fullerenes are potent ROS scavengers and there is evidence that ROS is involved in FcεRI signaling. Further, these test data demonstrate that selective inhibition of FcεRI mediator release from primary human MC/PBB using nano-engineered fullerene derivatives depends on the moieties added to the fullerene carbon cage. Moreover, fullerene derivatives have been shown to affect both phosphorylation of signaling molecules as well gene expression. Furthermore, these results show that appropriate fullerene derivatives may be effective treatments for diseases that are influenced by MC activation and may represent a new way to control MC responses before they occur.

Complete inhibition of FcεRI MC/PBB mediator release was not observed with TGA yet it was sufficient to improve disease outcomes. Previous studies showed that MC-targeting and complete inhibition of FcεRI mediator release in vitro is not necessary for in vivo efficacy.

To explore the in vivo effects of fullerene derivatives on MC responses, the degranulation/cytokine-blocking TGA and the cytokine blocking Inos on MC-induced anaphylaxis were tested. DNP-IgE-sensitized animals injected i.p. with DNP-BSA demonstrated a characteristic drop in core body temperature resulting from MC-driven anaphylactic shock.

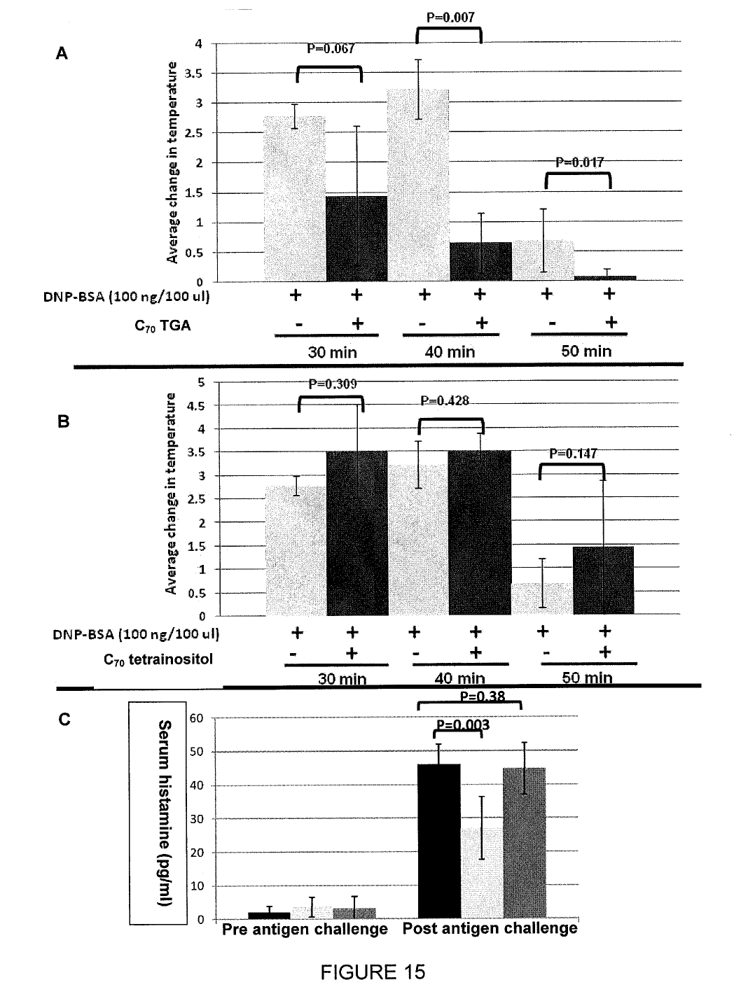

Mice (5 per group) were sensitized with 50 μg DNP-IgE in 100 μL PBS. Two hours later the mice were injected i.p. with PBS alone or 100 ng of fullerene derivative that inhibits degranulation and cytokine production, e.g., TGA (FIG. 15A) or cytokine production only, e.g., Inos (FIG. 15B) in 100 μL PBS. The following day rectal temperatures were recorded before mice were challenged i.p. with 100 μg of DNP-BSA in 100 μL PBS. Temperatures were recorded every 10 minutes (up to 50 minutes) following challenge with DNP-BSA. Increase in antigen-induced serum histamine release is blunted by fullerene derivative (FIG. 15C). Blood was collected from control (black bars), degranulation inhibiting fullerene derivatives (light gray bars) or cytokine inhibiting fullerene derivatives (dark gray bars) at 50 minutes and histamine content determined by ELISA.

When mice were injected with TGA before FcεRI challenge there was a significant reduction in the anaphylactic-induced drop in core body temperature and behavioral responses that accompany anaphylactic shock (FIG. 15A). However, Inos had no effect on MC-induced anaphylaxis (FIG. 15B). This suggests that the Inos interacts and/or indirectly inhibits a signaling molecule found in human MC FcεRI signaling and not in PBB. As expected, serum histamine levels were significantly lower in animals treated with the degranulation/cytokine inhibitor compared to controls (FIG. 15C). There was no significant increase in serum activity of ALT and AST between the untreated and fullerene treated mice injected with fullerene derivative concentrations 50 fold higher than that needed for in vivo efficacy (Table 7; tail vein or i.p. routes gave the same result). The injections were well tolerated and no change in behavior or body weights was noted. These experiments demonstrate that the efficacy of fullerene derivatives in vivo depends on how the carbon cage is derivatized and suggest they can be engineered at the nanoscale level to perform specific cellular functions.

TABLE 7
No liver toxicity is detected following FD injection

| | Day 2 | | | | | | Day 14 | | | | | |
|---|---|---|---|---|---|---|---|---|---|---|---|---|
| | Inositol | | | | | | Inositol | | | | | |
| | Treated | | TGA Treated | | Untreated | | Treated | | TGA Treated | | Untreated | |
| | n | Activity | n | Activity | n | Activity | n | Activity | n | Activity | n | Activity |
| Aspartate | 3 | 27.6 | 3 | 8.5 | 3 | 29.5 | 3 | 33.5 | 3 | 29.8 | 3 | 51.1 |
| aminotransferase (AST) | | (±2.1) | | (±2.3) | | (±9.2) | | (±4.2) | | (±9.9) | | (±4.1) |
| Alanine | 3 | 26.4 | 3 | 29.2 | 3 | 64.3 | 3 | 44.2 | 3 | 36.7 | 3 | 47.0 |
| aminotransferase (ALT) | | (±2.0) | | (±2.5) | | (±11.5) | | (±1.5) | | (±6.6) | | (±2.9) |

n = Number of Mice evaulated in Duplicates
Untreated = Normal Mice with PBS injection
Treated = Tail Vein injection of 100 μl of C₇₀-inositol or C₇₀-itetragylcolate

As discussed herein, no toxicity was observed using up to 100 μg/ml of fullerene derivatives—well above the concentrations in which efficacy was observed in vitro and in vivo. It is noted that thoroughly purified fullerene derivatives were employed in the studies, limiting the likelihood of confounding results due to sample impurities.

In separate experiments the in vivo administration (i.v. daily; 200 ng/three weeks) of TGA to mice showed no notable differences in behavior and there were no abnormalities observed upon gross pathological examination. No mutagenic potential (using the Ames test) was observed (not shown). No adverse reactions were observed in the anaphylaxis model when fullerene derivatives were injected and no liver damage was noted at the concentrations sufficient for in vivo efficacy. Taken together, the derivatives described herein are not cytotoxic to several cell lines tested and appear to have no acute in vivo cytotoxic effects.

Cultured monocytes will adhere to tissue culture flasks and differentiate into macrophages. Such cultured macrophages can be studied as a model system for foam cells.

### Cell Culture

The human monocytic cell line U937 was obtained from the American Type Culture Collection (Manassas, Va., USA). Cells are maintained in RPMI 1640 media enriched with 10% heat-inactivated FBS, 2 mM L-Glutamine, 10 mM HEPES buffer, 0.1 mM non-essential Amino Acids, 1% antibiotic/antimycotic, and 50 uM beta mercaptoethanol at 37° C., 6% CO₂.

### Foam Cell Differentiation

LDL from human plasma was oxidized as described by Kuzuya M, Yamada K, Hayashi T, et al. Oxidation of low-density lipoprotein by copper and iron in phosphate buffer. Biochim. Biophys. Acta 1991; 1084:198-201. To induce foam cell formation, the U937 monocytic cells were seeded at 10⁶ cells/mL in 24 well plates prior to experimental treatments. For differentiation into macrophage cells, the U937 cells were treated with 0.7 ug/mL phorbol myristilic acid (PMA) and incubated for 24 hours at 37° C., 6% CO₂. Oxidized-LDL (10 ug/mL) was added to the PMA-differentiated macrophage cells and incubated for 48 hours at 37° C., 6% CO₂.

### Determination of Foam Cell Formation Using Oil Red-O

Cells were fixed with 4% paraformaldehyde and Oil Red-O (ORO) staining was conducted for foam cell differentiated macrophages. Fixed and stained cell preparations were cytocentrifuged and washed in deionized H₂O for five minutes and then viewed under the microscope for detection of ORO stain as described in Koopman R, Schaart G, Hesselink M K., Optimisation of oil red O staining permits combination with immunofluorescence and automated quantification of lipids. Histochem. Cell Biol. 2001; 116:63-68.

### Flow Cytometry

Cells were recovered by centrifugation at 800×g at 4° C., washed with PBS/1% BSA, and blocked for 30 min at 4° C. with a 1/500 dilution of normal human serum. The cells were washed and incubated with either FITC-labelled anti-CD11b (10 ug/mL) or FITC-labelled Isotype Control (10 ug/mL) for one hour at 4° C. After three washes, cells were resuspended in 400 μl of PBS. The mean intensity of fluorescence was determined for at least 10,000 cells using a FACScan flow cytometer (BD Biosciences). All experiments were performed in duplicates.

Fullerene Effects on TNF-α Release from Macrophage Foam Cells

In order to observe the TNF-α release from foam cells, duplicate samples of cells (10⁷ cells/ml) were treated with or without fullerenes (5.0 μg/mL) for 24 hours. The next day cells were challenged with or without 0.7 μg/mL of PMA and varying concentrations of Ox-LDL (0.5 μg/mL, 1.0 μg/mL, 5.0 μg/mL, 8.0 μg/mL and 10.0 μg/mL) as described above. Controls samples were treated with 5.0 μg/mL ALM only and 0.7 μg/mL PMA only. Cells were placed in a 37° C. incubator and samples were taken at 12 and 24 hours. TNF-α levels were measured as previously described in Kepley C L. Antigen-induced reduction in mast cell and basophil functional responses due to reduced Syk protein levels. Int. Arch. Allergy Immunol. 2005; 138:29-39. When the assay was completed, the plate was read on a Biotek ELx800 plate reader and the resulting data was analyzed.

### Effects of ALM on U937 Monocyte Cell Viability

The effects of fullerenes on cell viability was examined in parallel with Vitamin C, a potent anti-oxidant (Table 8):

TABLE 8
% Viability

| | Day 3 | Day 6 | Day 9 |
|---|---|---|---|
| Untreated | 98.8% ± 0.20 | 93.9% ± 0.81 | 69.9% ± 0.90 |
| ALM (67 uM) | 98.2% ± 0.60 | 91.1% ± 0.33 | 67.0% ± 1.03 |
| ALM (6.7 uM) | 99.4% ± 0.56 | 90.9% ± 0.07 | 66.5% ± 0.03 |
| ALM (0.67 uM) | 98.3% ± 0.52 | 92.6% ± 2.31 | 67.2% ± 0.82 |
| Vitamin C (67 uM) | 98.2% ± 0.67 | 90.9% ± 0.07 | 60.7% ± 0.28 |
| Vitamin C (6.7 uM) | 97.4% ± 1.86 | 91.2% ± 0.33 | 61.8% ± 1.10 |
| Vitamin C (0.67 uM) | 98.8% ± 0.06 | 93.8% ± 1.09 | 60.9% ± 1.13 |

As seen in above Table 8, monocyte cells incubated with Vitamin C or ALM did not have toxic effects on the viability of serum-starved cell. No significant differences in cell viability was observed using up to 100 ug/mL ALM compared to control cells at days six and nine. Similar results were obtained with the monocytic cell line THP-1 and monocytes derived from whole blood (data not shown).

### Fullerenes Prevent Lipid Accumulation in Macrophage Foam Cells

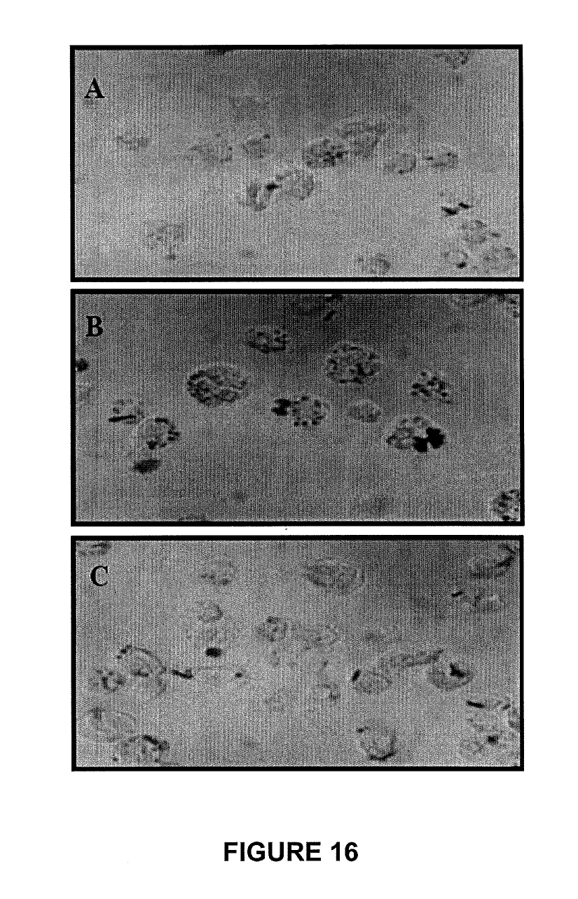

Differentiated monocytes were used to observe the effects that ALM would have on lipid uptake. Untreated U937 monocytes showed very little ORO staining (FIG. 16A). Analysis of lipid uptake in cells incubated with PMA and various concentrations of Ox-LDL showed a significant amount of accumulation in the cytoplasms of cells as seen in FIG. 16B. However, cells pre-incubated with ALM prior to the addition of PMA and Ox-LDL had significantly less staining than those not receiving ALM, indicating less lipid accumulation (FIG. 16C). Dose response studies demonstrated that 5 μg/mL ALM for 24 hours was optimal and this concentration was used in all subsequent experiments (data not shown).

### Fullerenes Inhibit Foam Cell Adhesion Via Mac1 Down Regulation

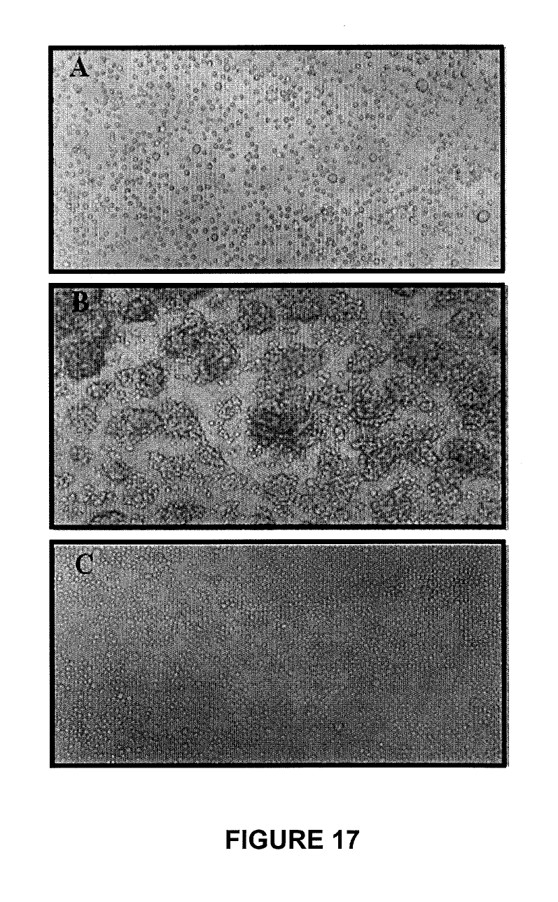

To determine if fullerene derivatives affect the foam cell formation process where activated monocytes initiate cellular clumping as part thereof, equal numbers of U937 monocytes were separated into three different groups. One group of cells was treated with 0.7 μg/mL PMA for 24 hours while another was pre-treated with 5 μg/mL ALM for 24 hours before receiving PMA. The control group did not receive PMA or ALM. After the appropriate time had elapsed, the PMA-treated cells showed significant clumping (FIG. 17). However, the ALM-treated cells showed no significant clumping.

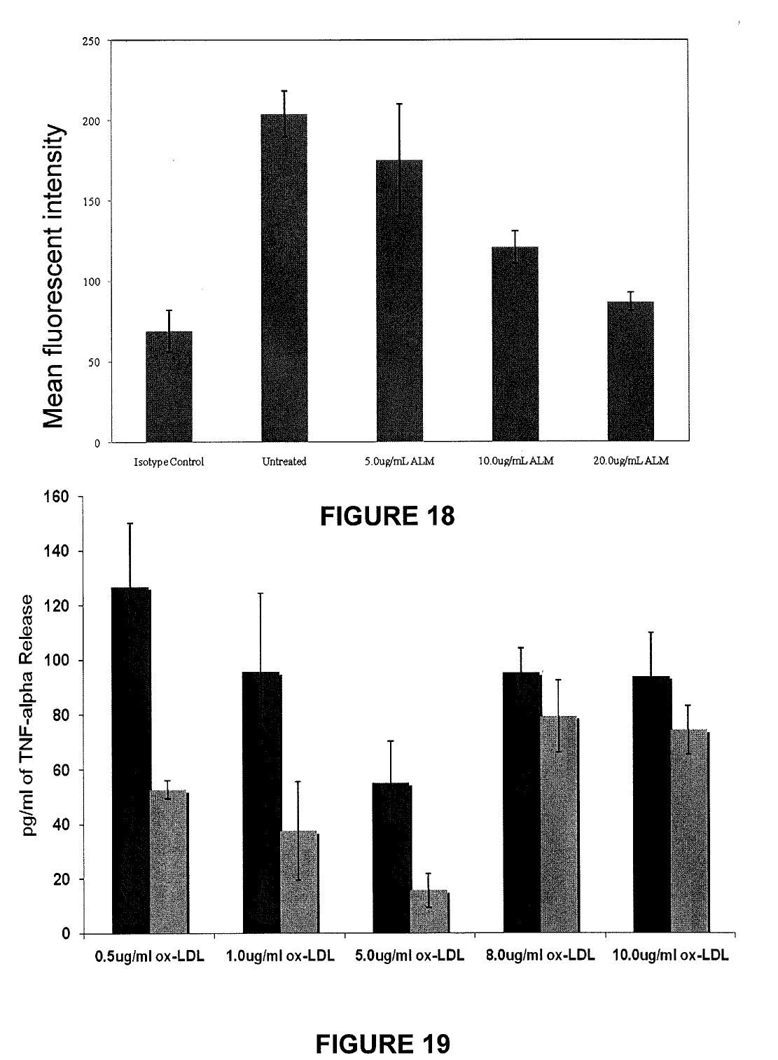

To test whether the fullerene derivatives inhibit the clumping of activated monocytes through Mac1 inhibition, cells were treated as above and examined for the upregulation of Mac1 using FACs analysis. As seen in FIG. 18, the same fullerene treatment conditions that prevented cell clumping also prevented the upregulation of Mac1. Thus, fullerenes prevent activation-induced monocytic cell-cell adhesion possibly through the inhibition of Mac1.

### Preincubation of Monocytes Prevents TNF-α-Induced Foam Cell Formation

To test whether fullerenes inhibited the induction of foam cell formation in part through the inhibition of TNF-α, the effects ALM had on TNF-α release from activated monocytes were examined. As shown in FIG. 19, cells treated for 24 hours with 0.7 μg/mL of PMA and any concentration up to 8 μg/mL of Ox-LDL released a significantly higher amount of TNF-α than untreated monocytes. However, when cells were pretreated with 5.0 μg/mL of ALM, there was a statistically significant decrease in the amount of TNF-α released ranging from 58% to 77% inhibition. Similar statistically significant inhibition of release was seen with ALM when levels were monitored at 12 hours (data not shown). Thus, ALM may inhibit foam cell formation through reductions in TNF-α levels.

The results showed that cells pretreated with the fullerenes had a significant decrease in total ORO staining in the cell as opposed to those that were untreated. Current treatment for atherosclerosis is the management of lipid accumulation and several trials have demonstrated reduced cardiovascular events and mortality with lipid-lowering therapy. Ashen M D, Blumenthal R S. Clinical practice. Low HDL cholesterol levels. N. Engl. J. Med. 353:1252-1260 (2005). It is assumed that the reduction in lipids results in the reduction in foam cells that line arteriole walls. Thus, fullerenes may be a new approach for lowering a patient's lipid burden and subsequent plaque accumulation.

There are several studies which support the importance of the monocyte to macrophage differentiation in the initiation and progression of atherosclerosis. Davis S C, Ricotti C, Cazzaniga A, Welsh E, Eaglstein W H, Mertz P M, Microscopic and physiologic evidence for biofilm-associated wound colonization in vivo. Wound. Repair Regen. 16:23-29 (2008). One of the first events of atherogenesis is invasion of the arterial wall by monocyte derived macrophages. Monocytes are further induced toward foam cell formation through the induction of cellular adhesion molecules that mediate their adhesion to vessel walls. Galkina E, Ley K., Vascular adhesion molecules in atherosclerosis. Arterioscler. Thromb. Vasc. Biol. 27:2292-2301 (2007). While there are several steps involved in leukocyte trafficking into vascular tissues ICAM-1 is particularly involved in atherosclerosis through the regulation of monocyte recruitment (which express Mac1; a ligand for ICAM) into atherosclerosis-prone areas. In atherosclerosis Ox-LDL induces endothelial ICAM-1 upregulation which would provide more opportunities for interactions with monocytic Mac1. Several other studies in humans and mice suggest that ICAM-1-Mac1 interactions participates in the initial adhesion of monocytes/macrophages onto vessels. Martineau L, Davis S C., Controlling methicillin resistant Staphyloccocus aureus and Pseudomonas aeruginosa wound infections with a novel biomaterial. J. Invest Surg. 20:217-227 (2007). The studies here demonstrate that fullerenes reduce cellular adhesion in monocytic cells through reduction of CD11 b expression. These results suggest fullerenes may block the initial steps involved in atherosclerosis—the adhesion of monocytes to blood vessels—by preventing the upregulation of adhesion receptors on activated monocytes.

Inflammation plays a vital role in all phases of atherosclerosis. Hansson G K, Robertson A K, Soderberg-Naucler C., Inflammation and atherosclerosis. Annu. Rev. Pathol. 1:297-329 (2006). This inflammation is a result of the stimulation of monocyte and macrophage cells: both of which release pro-inflammatory cytokines: IL-1β, IL-6, and TNF-α, which has been shown to have a profound influence on the exacerbation of atherosclerosis. These cytokines are often observed in the atherosclerotic lesions. Studies examining the suppression of TNF-α (mostly from rheumatoid arthritis trials) suggest that anti-TNF-α therapy seems to be, at least in part, associated with concomitant reduction of the risk of cardiovascular events. Avouac J, Allanore Y., Cardiovascular risk in rheumatoid arthritis: effects of anti-TNF drugs. Expert. Opin. Pharmacother. 9:1121-1128 (2008). Applicants' findings show that with varying concentrations of fullerenes the production of TNF-α could be reduced in some cases as much as 72%. These differences of TNF-α release may be important in providing a new way to prevent atherosclerosis.

Several studies have demonstrated that the induction of foam cell formation in atherosclerosis is mediated in part through oxidative stress suggesting anti-oxidant therapy may be beneficial for preventing this process. Iuliano L., The oxidant stress hypothesis of atherogenesis. Lipids 36 Suppl:S41-S44 (2001). For example the mechanism for lipid accumulation in monocyte cells is governed by the CD36 and SRA receptors. Furthermore, oxidative stress increases CD36 expression. Fuhrman B, Volkova N, Aviram M., Oxidative stress increases the expression of the CD36 scavenger receptor and the cellular uptake of oxidized low-density lipoprotein in macrophages from atherosclerotic mice: protective role of antioxidants and of paraoxonase. Atherosclerosis 161:307-316 (2002). Previous reports further support the hypothesis and have proposed that membrane expression of CD36 involves redox signaling pathway via NADPH oxidase activation and the administration of antioxidants leads to a reduction in CD36 expression in monocytes derived from humans. Given that fullerenes are extremely potent anti-oxidants (Wilson S R, Schuster D I, Nuber B, Meier M, Prato M, Taylor R., Fullerenes: Chemistry, Physics, and Technology. K. Kadish, and R. Ruoff, eds, John Wiley & Sons, NY, 2000), it is tempting to speculate that the derivatives described here may be exerting their inhibitory effects through the inhibition of oxidative stress.

The results show that fullerenes exert their anti-atherogenic effects by inhibiting the formation of foam cell formation and adhesion through the reduction of inflammatory cytokine release and adhesion molecule membrane expression. These results further extend the utilization of fullerenes and suggest they may represent a novel therapeutic candidate for the treatment of atherosclerosis.

The present disclosure relates to use of any one or more of the fullerenes described herein for the treatment of an inflammatory disease or for inhibiting the build-up of arterial plaque. The present disclosure also relates to the use of any one or more of the fullerenes described herein for manufacture of a medicament, particularly the manufacture of a medicament for treating inflammatory disease or for inhibiting the build-up of arterial plaque.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed. All publications, patents, patent applications and other references cited herein are hereby incorporated by reference.

While the disclosure has been described in detail with reference to certain embodiments thereof, it will be apparent to one skilled in the art that various changes can be made, and equivalents employed, without departing from the scope of the disclosure. In addition, the following examples are illustrative of the methods described herein and should not be considered as limiting the foregoing disclosure in any way.

### EXAMPLES

### Example 1

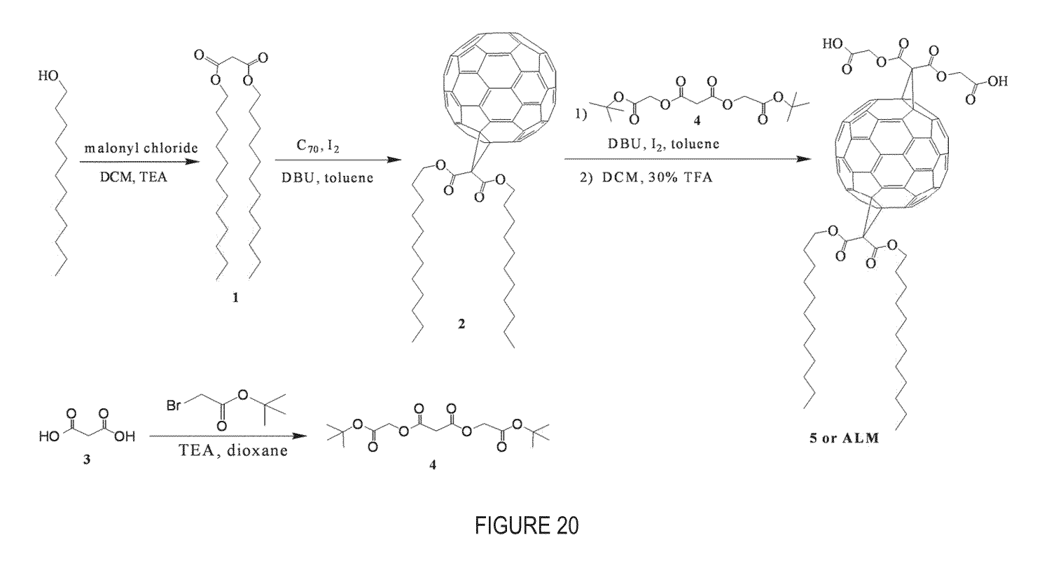

### Preparation of Amphiphilic Fullerene Derivative Depicted in FIG. 20

### Compound 5; Labeled LnW0042 in FIG. 6

### Step 1. Synthesis of Didodecylmalonate (Compound 1 in FIG. 20)

10 mmole dodecyl alcohol was dissolved in 30 mL dry dichloromethane (DCM), to which 12 mmole triethylamine (TEA) was added and stirred under nitrogen atmosphere. Then, 5 mmole malonyl chloride was dissolved in 1 ml dry DCM, and dropwise added to the above solution within a period of 10 minutes. Upon completion of the addition of malonyl chloride, the reaction mixture was stirred for a few hours and monitored by TLC. When reaction was completed, the reaction mixture was washed with brine twice and the combined organic phase was dried over MgSO₄, filtered, and concentrated to 1-2 mL. Flash column with silica gel was used to purify the products with DCM as the solvents. Yield: 85%.

### Step 2. Synthesis of C₇₀ Monoadduct (Compound 2 in FIG. 20)

840 mg (1.0 mmole) of C₇₀ was dissolved in 50 mL o-xylene and sonicated for 3 minutes, and then 400 mL toluene was added. Next, 1.0 mmole of the malonate 1 was added and the whole mixture was stirred, to which 1.0 mmole of iodine (MW=254 Da) was added. After stirred for 10 minutes, a 20 mL toluene solution of DBU (2.5 mmole, MW=151 Da, 1,8-diazabicyclo[5.4.0]undec-7-ene) was added to the mixture over a period of 15 minutes, and stirred for two hour. TLC monitored the reaction progress with 3:7 toluene/hexanes. Upon completion of the reaction, the product was concentrated to 10 mL (not to dryness) and 30 mL hexanes were added to dilute. Next, the mixture was loaded to the top of a silica gel column for purification. A mixture of solvents of 3:7 toluene/hexanes was used to elute unreacted C₇₀ (first band), and then the product (second band), which was then rotavaped and then pumped under vacuum for overnight before NMR and MALDI-MS. Yield: 60%.

### Step 3. Synthesis of Compound 5

127.8 mg (0.1 mmole) of C₇₀ monoadduct 2 (MW=1278) was dissolved in 60 mL toluene. Next, 0.1 mmole of the malonate 4 (see detailed synthesis in Example 2) was added and the whole mixture was stirred, to which 0.1 mmole of iodine (MW=254 Da) was added. After stirred for 15 minutes, a 10 mL toluene solution of DBU (0.25 mmol, MW=151 Da) was added to the mixture over a period of 10 minutes, and stirred for two hour. TLC monitored the reaction progress with DCM or toluene/EA (98:2). When complete, the mixture was concentrated to 10 mL (not to dryness), and loaded to the top of a silica gel column for purification. Toluene was used first to elute unreacted C₇₀ monoadduct (first band), and then the product (second band), which was then rotavaped and then pumped under vacuum for overnight before NMR and MALDI-MS. Yield: 60%. The obtained the tert-butyl ester of ALM was dissolved in DCM and TFA (v:v 3:1) at 20 mg/mL and stirred at RT for 6 hours. Solvents were evaporated and dried under vacuum for overnight to quantitatively yield the final product ALM, which was characterized by MALDI-MS and NMR.

### Example 2

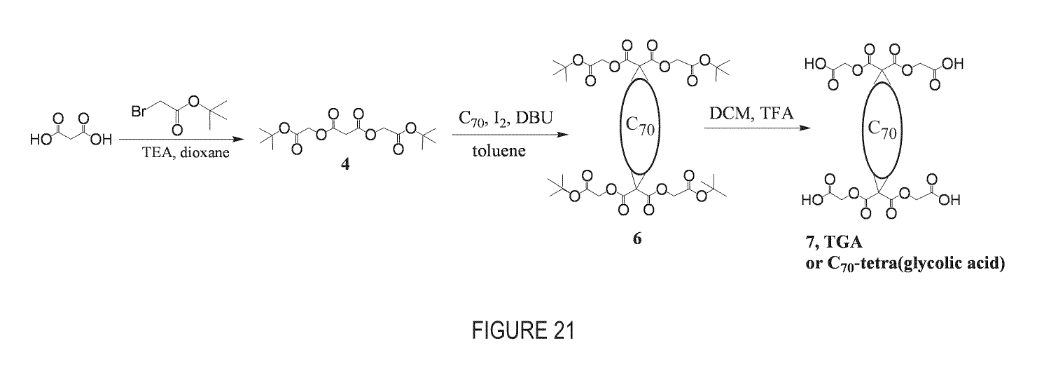

### Preparation of C₇₀-Tetraglycolic Acid Depicted in FIG. 21

### TGA or Compound 7; Labeled “LnW0048” in FIG. 5

### Step 1. Synthesis of di(tert-butylacetoxy)malonate (Compound 4 in FIG. 21)

To a solution of malonic acid (40.0 mmol, 4.16 g) in dioxane was added 11.1 mL TEA (80.0 mmol). The mixture was stirred for 30 minutes, and then 11.8 mL (80.0 mmol) of tert-butyl bromoacetate in 12 mL dioxane was added and stirred over weekend. TLC monitored the reaction progress until completion. The precipitate was filtered and washed with ether. The filtrate was then washed with brine twice, dried over MgSO₄, and concentrated for NMR analysis. Yield: 75%.

### Step 2. Synthesis of C₇₀ Tetraglycolic Acid Tert-Butyl Ester (Compound 6 in FIG. 21)

840 mg (1.0 mmole) of C₇₀ was dissolved in 50 mL o-xylene and sonicated for 3 minutes, and then 200 mL toluene was added. Next, 2.0 mmole of the malonate 4 was added and the whole mixture was stirred, to which 2.0 mmole of iodine was added. After stirred for 10 minutes, a 20 mL toluene solution of DBU (5.0 mmole, MW=151 Da, 1,8-diazabicyclo[5.4.0]undec-7-ene) was added to the mixture over a period of 15 minutes, and stirred for 8 hours. TLC was used to monitor the reaction progress. When complete, it was concentrated to 40 mL and loaded onto the top of a silica gel column for purification. Toluene was used to remove unreacted C₇₀ and then DCM was to remove the monoadduct. Finally a mixture of EA and DCM was to elute the product, which was rotavaped and vacuum pumped for overnight for NMR and MALDI-MS. Yield: 70%.

### Step 3. Synthesis of C₇₀ Tetraglycolic Acid (Compound 7)

The tert-butyl ester precursor 6 was dissolved in DCM at 20 mg/mL, and equal volume of TFA was added, and stirred overnight. TFA was removed by evaporation and water washing. The DCM layer was then dried to give pure TGA product. Yield: quantitative.

### Example 3

### Preparation of C₇₀-Tetrainositol

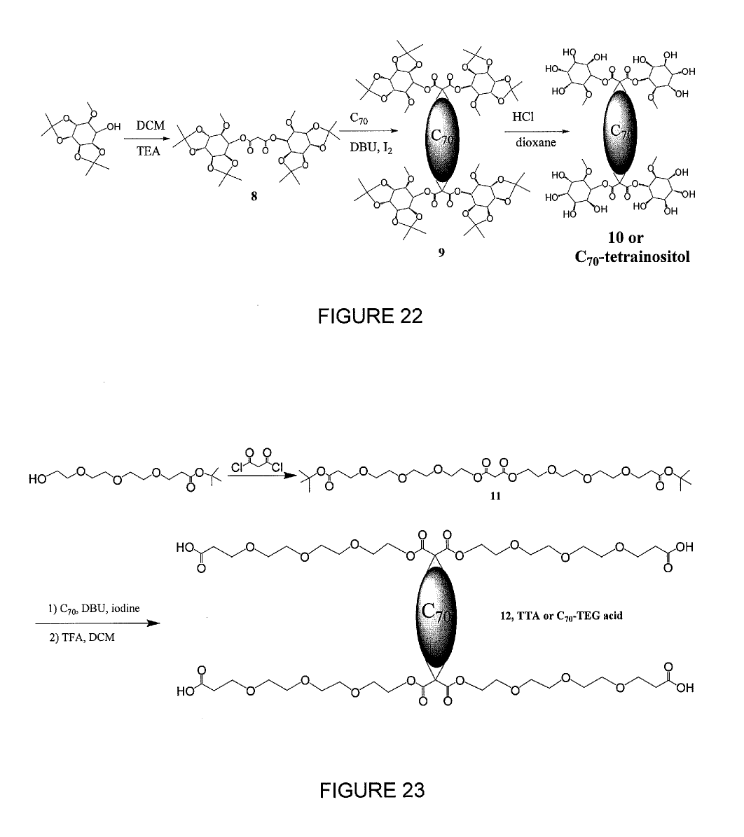

### Compound 10 in FIG. 22

### Step 1. Synthesis of Protected Inositol Malonate (Compound 8 in FIG. 22)

10 mmole 1,2;5,6-bis-O-(1-methylethylene)-3-methyl-1D-chiro-inositol was dissolved in 50 mL dry DCM, to which 12 mmole TEA was added and stirred under nitrogen. Then, 5 mmole malonyl chloride was dissolved in 1 ml dry DCM, and dropwise added to the above solution within a period of 10 minutes. Upon completion of the addition, the reaction mixture was stirred for 6 hours. When completed, the reaction mixture was washed with brine twice and the combined organic phase was dried over MgSO₄, filtered, and concentrated to 1-2 mL. Flash column with silica gel was used to purify the products with 20% EA in DCM as the solvents. The product was colorless viscous liquid. Yield: 55%. Proton and carbon NMR confirmed the structure.

### Step 2. Synthesis of C₇₀-Tetrainositol-Acetal Protected (Compound 9 in FIG. 22)

84 mg (0.1 mmole) of C₇₀ was dissolved in 60 mL toluene. Next, 0.2 mmole of the malonate 8 was added and the mixture was stirred, to which 0.2 mmole of iodine was added. After stirred for 15 minutes, a 10 mL toluene solution of DBU (0.50 mmol) was added to the mixture over a period of 10 minutes, and stirred for 6 hours. When it was complete, the reaction mixture was concentrated to 10 mL and loaded to the top of a silica gel column for purification. DCM was used first to elute unreacted C₇₀ and its monoadduct and then solvent was changed to DCM/EA to elute the product, which was rotavaped and pumped under vacuum for overnight before NMR and MALDI-MS.

### Step 3. Synthesis of C₇₀-Tetrainositol (Compound 10 in FIG. 22)

50 mg of the obtained octakis-acetal protected precursor compound 9 was dissolved in 20 mL 4.0M hydrochloride solution in dioxane. A few drops of water was added and stirred for 30 hours. Complete deprotection was achieved. Solvent were removed completely and dried under vacuum overnight to yield pure final product, with NMR and MALDI-MS data confirmed.

### Example 4

### Preparation of C₇₀-TEG Acid

### Compound 12 or TTA FIG. 23

### Step 1. Synthesis of Di(Tert-Butyl TEG Propionate) Malonate 11

10 mmole tert-butyl 12-hydroxy-4,7,10-trioxadodecanoate was dissolved in 50 mL dry DCM, to which 12 mmole TEA was added and stirred under nitrogen. Then, 5 mmole malonyl chloride was dissolved in 1 ml dry DCM, and dropwise added to the above solution within a period of 10 minutes. Upon completion of the addition, the reaction mixture was stirred for 4 hours. When completed, the reaction mixture was washed with brine twice and the combined organic phase was dried over MgSO₄, filtered, and concentrated to 1-2 mL. Flash column with silica gel was used to purify the products with DCM/EA as the solvents. Yield: 85%. Proton and carbon NMR confirmed the structure.

### Step 2. Synthesis of C₇₀-TEG Acid 12

84 mg (0.1 mmole) of C₇₀ was dissolved in 60 mL toluene. Next, 0.2 mmole of the malonate 11 was added and the mixture was stirred, to which 0.2 mmole of iodine was added. After stirred for 15 minutes, a 10 mL toluene solution of DBU (0.50 mmol) was added to the mixture over a period of 10 minutes, and stirred for 6 hours. When it was complete, the reaction mixture was concentrated to 10 mL and loaded to the top of a silica gel column for purification. DCM was used first to elute unreacted C₇₀ and its monoadduct and then solvent was changed to DCM/EA to elute the product, which was rotavaped and pumped under vacuum for overnight before NMR and MALDI-MS. Yield: 70%. The obtained tert-butyl ester of C₇₀ TEG acid was dissolved in DCM and TFA (v:v 3:1) at 20 mg/mL and stirred at RT for 8 hours. Solvents were evaporated and dried under vacuum for overnight to quantitatively yield the final product C₇₀ TEG acid, which was characterized by MALDI-MS and NMR.

### Example 5

### Preparation of Phenylpropionic Acid-Triazole-Mixed Malonateamide-C₇₀-TEG-COOH

### Compound 19 (FIG. 24)

### Step 1. Synthesis of 2-(4-azidomethylphenyl)-propionic acid (compound 14 in FIG. 24)

To a solution of 2-(4-bromomethylphenyl)-proprionic acid (1.5 g) in 1,4-dioxane was added NaN₃ (5 g) and 15-crown-5 (100 mg). The mixture was heated to 80° C. for 16 h, then cooled. The solvent was removed under reduced pressure, and the residue chromatographed on silica to yield 2-(4-azidomethylphenyl)-proprionic acid as an off white solid.

### Step 2. Preparaton of Acetylene Malonate (Compound 15)

To a separate solution of ethoxy malonyl chloride was added triethylamine and propargyl amine in equal molar equivalents. The mixture was stirred 20 minutes and purified by column chromatography.

### Step 3. Preparation of Triazole Propionic Acid (Compound 16)

A mixture of the acetylene malonate (1 equivalent), 2-(4-azidomethylphenyl)-propionic acid (1 equivalent), CuSO₄ (5 mol %), triethylamine (5 equivalents), and ascorbic acid (50 mol %) was stirred in 1:1 THF:water overnight following a procedure widely used in the literature. Solvents were removed under reduced pressure and the residue was extracted with ethyl acetate and water. The ethyl acetate layer was further purified by column chromatography to yield the triazole product.

### Step 4. Preparation of bis(tert-butyl hydroxy trioxadodecanoate (Compound 17)

bis(tert-Butyl 12-hydroxy-4,7,10-trioxadodecanoate)malonate was synthesized by reaction with I₂, DBU, and C₇₀ in xylene, followed by column chromotography purification.

### Step 5. Preparation of Triazole Propionic Acid Malonamide (Compound 18)

Using the C₇₀ mono-adduct from above, I₂, DBU, and the triazole-containing malonamide, a second adduct were added to the C₇₀ cage. After 20 minutes of reaction under nitrogen at room temperature, the reaction was poured onto a silica column and purified by chromatography.

### Step 6. Synthesis of Compound 19

Finally, deprotection of the t-butyl esters was achieved by reaction with trifluoroacetic acid in CH₂Cl₂ (1:1) overnight. Volatiles were removed under reduced pressure to yield the product, 19.

### Example 6

### Preparation of C₇₀-Tetraniacin

### Step 1. Synthesis of C₇₀-Tetrabromide

C₇₀ (84 mg) was dissolved in anhydrous toluene (50 mL) and the mixture was sonicated for 2 minutes, to which 2-bromoethyl malonate (63.2 mg) and iodine (50.8 mg) was added and stirred for 5 minutes under argon. A toluene solution of DBU (75.5 mg in 10 mL anhydrous and deoxygenated toluene) was added dropwise. The reaction mixture was stirred for additional 5 hours before it was washed with brine. The toluene solution was dried, concentrated and subjected to silica gel column for purification of the c₇₀-tetrabromide using toluene as the eluant. The final compound was dried in vacuum and characterized by MALDI and NMR.

### Step 2. Synthesis of C₇₀-Tetraniacin

C₇₀-tetrabromide (50 mg) was reacted with excessive niacin (2 g) (MW=123.1 g/mol, m.p. 235° C.) at 240° C. for 6 hours in a pre-dried flask under argon protection. After the reaction was done, the mixture was cooled to room temperature and dissolved in a dilute bicarbonate basic water (pH=9.0). The mixture was filtered to remove undissolved materials, and the filtrate was thoroughly dialyzed with a 1000 MWCO membrane to remove unreacted niacin.

The dialyzed products were further purified by crystallization. In brief, the dialysate containing the products was adjusted to neutral pH and then concentrated and isopropyl alcohol was slowly added until precipitation occurs. After the resulting mixture was placed in fridge for 10 hours, the precipitates were collected and dried in a vacuum oven. Alternatively, C₇₀-tetraniacin can be synthesized by reacting C₇₀-tetrabromide (50 mg) with ethyl pyridine-3-carboxylate (ethyl niacin, 5 mL) at 150° C. for overnight. After cooled to room temperature, precipitates were collected and washed with cold ethanol. The precipitates were then dried and hydrolyzed in basic water to remove the four ethyl groups to afford the C₇₀-tetraniacin. The final product was characterized by NMR, IR and UV-Vis.

### Example 7

### Preparation of C₇₀—(OH)₁₂

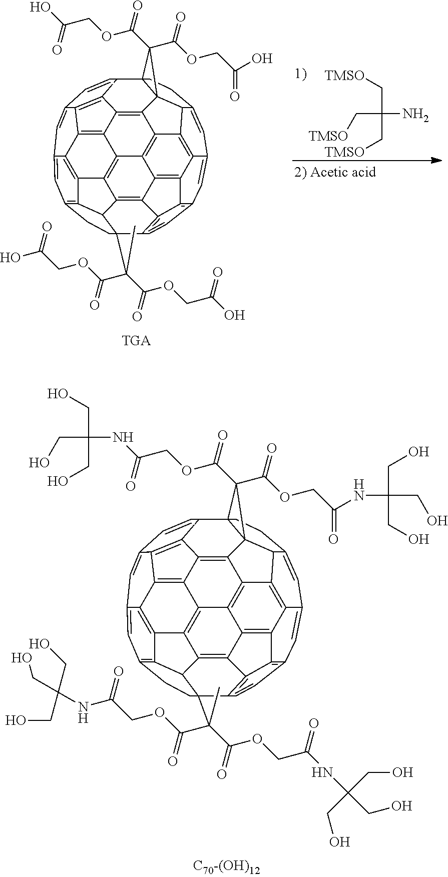

A solution of NHS (23 mg) and DCC (41 mg) in dry DMF was added to a solution of TGA (28 mg) in dry DMF (5 mL), and stirred for 8 hours. TLC shows complete conversion to NHS esters. TMS-protected TRIS in large excess was added to the above mixture and stirred overnight. All solvents were evaporated under vacuo and the residue was reconstituted in ethyl ether and filtered. This step was repeated 3 times until all byproducts were removed. The ether solution was dried and redissolved in a mixture of THF/acetic acid/water and stirred overnight. THF and acetic acid were rotavaped in vacuo, and the product was collected, washed with THF and dried. Yield is 75%. The product was characterized by MALDI and NMR (d6-DMSO as solvent).

### Example 8

### Preparation of C₇₀-(DMAE)₄

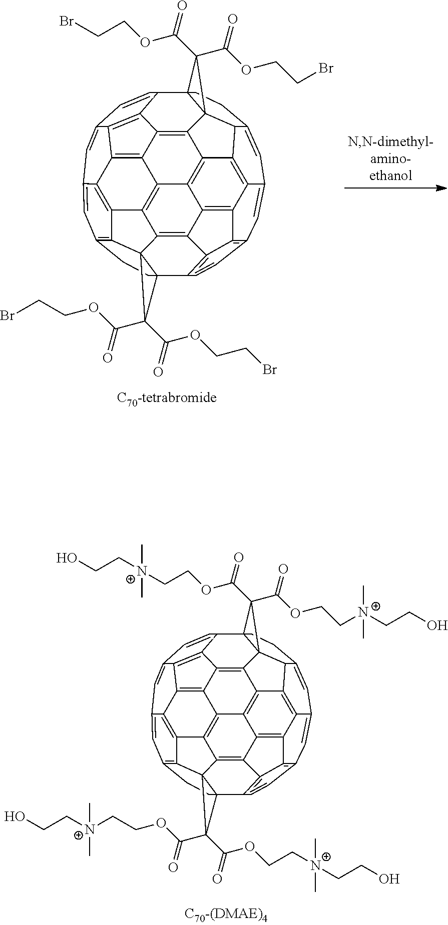

C₇₀-tetrabromide (10 mg) was dissolved in deoxygenated DMF (5 mL) and deoxygenated N,N-dimethylaminoethanol (10 mL) was added. The mixture was completely deoxygenated with argon for 30 minutes and reacted 28 hours at 80° C. After the reaction was complete, the mixture was cooled and dialyzed against DI water with MWCO of 1000 to remove any non-fullerene materials. The dialyzed product was dried and characterized by NMR, UV-Vis and MALDI. UV-Vis shows the characteristic absorption peaks of C₇₀ bisadduct in the range of 400-550 nm, showing no reaction occurred between the C₇₀ fullerene cage and tertiary amines under the employed conditions. Quantitative yield was obtained. The product has moderate solubility in buffer.

### Example 9

### Preparation of C₇₀—(PC)₄

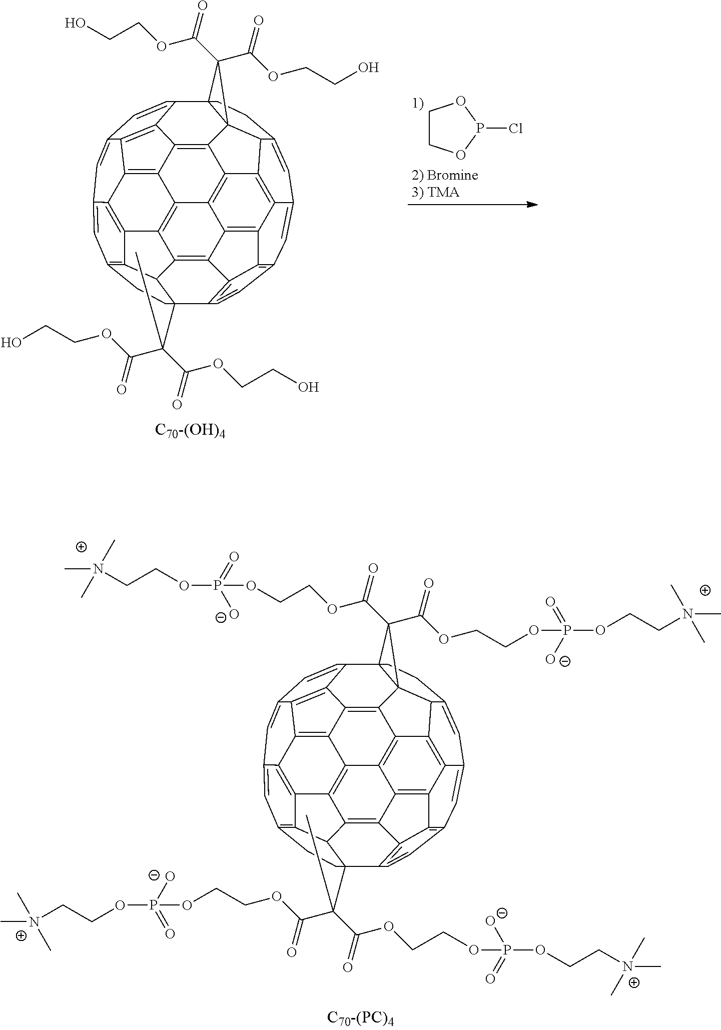

To a solution of C₇₀—(OH)₄ (0.7 mmol) which was made reacting C₇₀ with bis(2-hydroxylethyl)malonate (2 equivalents) under typical Bingel conditions followed by chromatographic purification, in THF (20 mL) cooled with salt-ice bath was added DIPEA (0.5 mL, 2.8 mmol) and ethylene chlorophosphite (0.2 mL, 2.1 mmol). The reaction mixture was stirred for 90 minutes and then neat bromine (0.1 mL, 2.1 mmol) was added. After 30 min, water (5 mL) was added and stirred with the temperature slowly rising to RT after salt-ice bath was removed. Then DCM (20 mL) was added and phase separated. The organic phase was rotavapored to dryness. The residue was then reconstituted in 1:1 DCM and isopropanol. 40% trimethylamine (17 mmol) solution (1 mL) was added and stirred for 2 hours at 0° C. The mixture was stirred at RT for another 12 hours and concentrated. The residue was chromatographed on silica gel with DCM/EA/CH₃OH as the solvent system. The product was eluted as the third band after unreacted and intermediate fullerene compounds were removed. NMR shows singlet peak at 3.1-3.2 ppm corresponding to the three methyl groups on the quaternary amine as well as methylene protons adjacent to the malonate ester and phosphate esters in the region of 4.1-4.5 ppm.

### Example 10

### Preparation of C₇₀-Tetrasulfonate

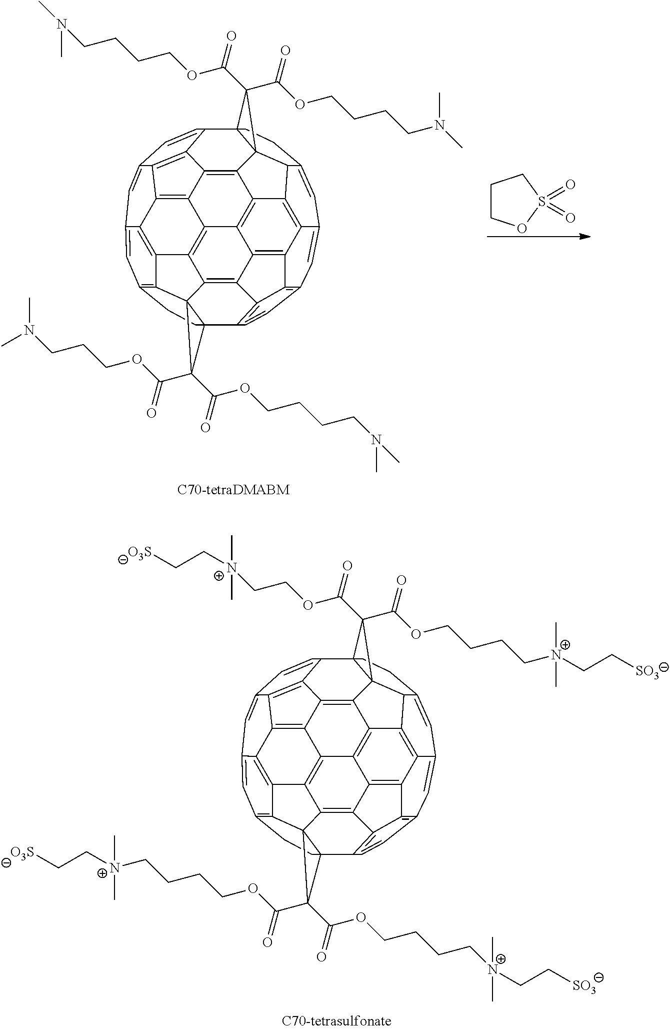

C₇₀-tetraDMABM was made by reacting C₇₀ with N,N-dimethylaminobutyl malonate (DMABM) (2 equivalents), iodine (2 equiv.) and DBU (Diaza(1,3)bicyclo[5.4.0]undecane) (5 equiv.) in toluene under typical Bingel conditions and purified with neutral alumina column. C₇₀-tetraDMABM (0.1 mmol) was subsequently refluxed with excessive 1,3-propane sulfone (0.8 mmol) overnight in a mixture of DMF and water. Excessive sulfone was destroyed by adding more TEA and stirring for 2 hours at RT. The final material were purified by dialysis with 1000 MWCO regenerated cellulose tubes against PBS solution, to remove all small molecule reactants or impurities. The product shows characteristic UV-V is absorption peaks of C₇₀ bisadducts, and NMR spectrum conforms to the Zwitterionic structure.

### Example 11

### Preparation of C₇₀-Tetrapyridine

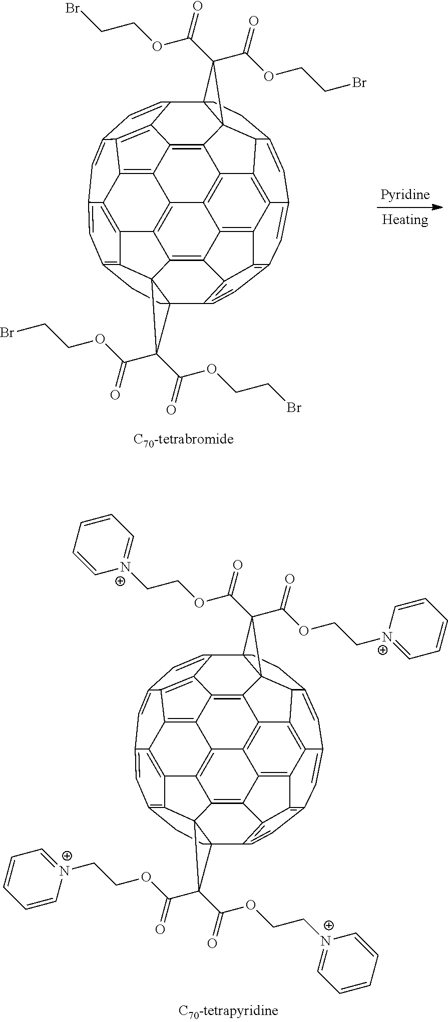

C₇₀-tetrapyridine was synthesized by reacting C₇₀-tetrabromide (50 mg) with neat pyridine (5 mL) at 70° C. for 21 hours in a pressure tube reactor, and the tube remained tightly closed during the reaction period. After the reaction was complete, the majority of fullerene materials precipitate onto the bottom of the tube. The mixture was centrifuged to remove pyridine solution with light reddish colors, and the precipitates were washed 3 times with toluene and ether respectively, and then dried in vacuo for 6 hours. The product was readily dissolved in both DI water and phosphate buffered saline with solubility of >2 mg/mL. NMR spectrum in D₂O revealed the presence of aromatic protons of pyridine with low-field shifts of 0.2-0.6 ppm due to the quaternization of the pyridine nitrogen atom.

### Example 12

### Preparation of C₇₀-Glu

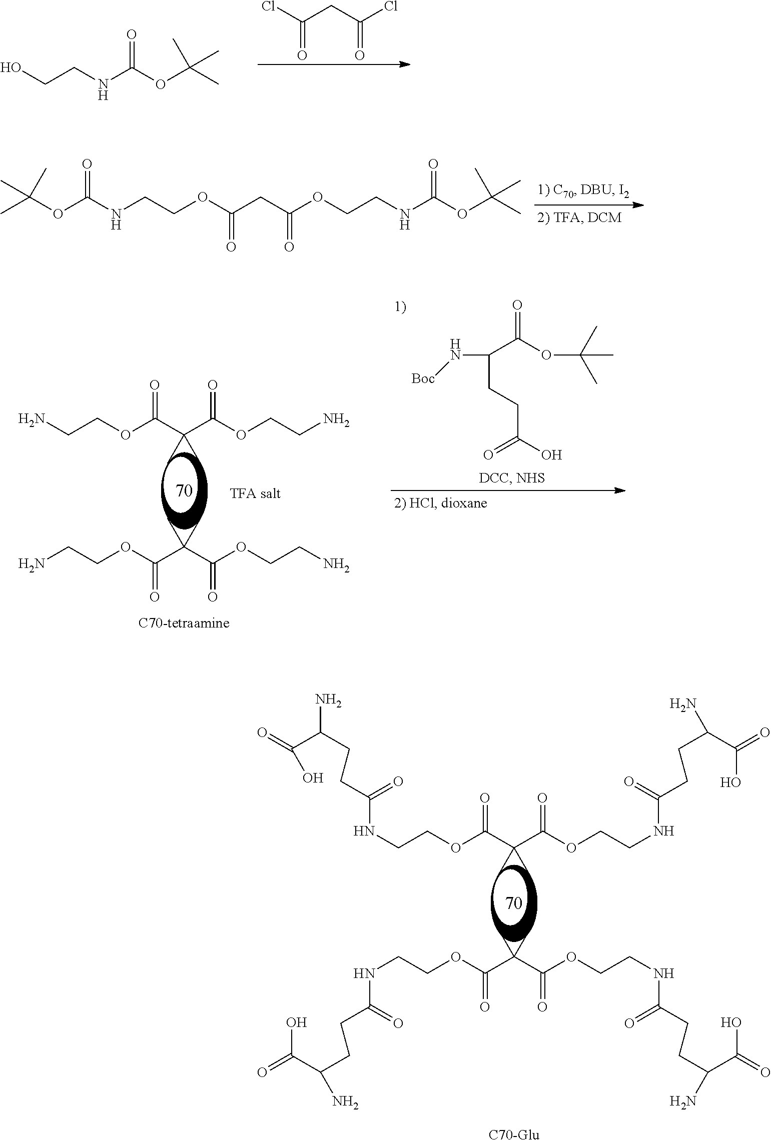

C₇₀-tetraamine building block was synthesized by reacting C₇₀ with Boc-aminoethyl malonate (2 equivalents), iodine (2 equiv.), and DBU (5 equiv.) in toluene at RT for 7 hours. The product was purified on silica gel column with DCM/EA as the eluant, followed by acidic removal of the Boc groups in a mixture of 20% TFA in DCM for 4 hours at RT to quantitatively yield the TFA salt of the C₇₀-tetraamine compound, which (0.1 mmol, neutralized with DIPEA) was subsequently added to and reacted with a premade solution of Boc-Glu-OBut (0.5 mmol), DCC (0.5 mmol and NHS (0.5 mmol) in anhydrous THF. The mixture was stirred overnight and the desired product was purified by silica gel column using DCA/EA/MeOH as the solvent. The product was characterized by MALDI-MS and NMR. It was then re-dissolved in a 4.0M HCl dioxane solution to remove the Boc and tert-butyl protecting groups to generate compound C₇₀-Glu. NMR of the final compound confirmed the complete removal of all Boc and butyl groups. This method can be used to synthesize a number of C₇₀ amino acid derivatives starting with different partially protected natural amino acids which can be obtained from Advanced Chemtech Inc.

### Example 13

### Preparation of C₇₀-Tetraphosphate

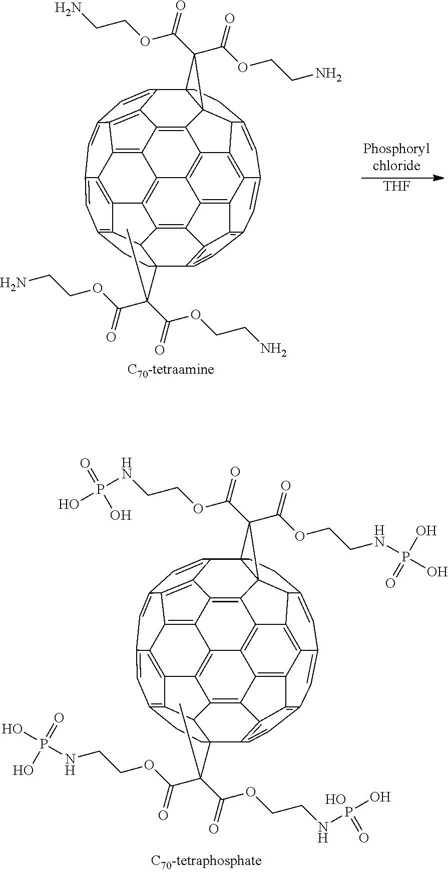

TFA salt of the C₇₀-tetramine compound (0.1 mmol) was dissolved in anhydrous TFA, and neutralized with DIPEA (diisopropylethylamine) (0.4 mmol). The in situ generated amine groups were reacted with phosphorous oxychloride (phosphoryl chloride) (50 mmol) for 4 hours at RT under argon. After the reaction was completed, the reaction mixture was cooled with a salt/ice bath and water was dropwise added to neutralize all unreacted phosphorous chloride and convert the remaining P—Cl bonds in the product to P—OH. The final product was dissolved in basic carbonate solution and dialyzed with MWCO 1000 for 8 hours to remove phosphoric acid salts. The final product can be dissolved in slightly basic aqueous solution and it remained dissolved when the pH was adjusted to 7.4. NMR shows the conversion of amines to phosphamide.

While various embodiments have been particularly shown and described herein, it will be understood by those skilled in the art that various changes in form and details may be made therein without departing from the spirit and scope of these embodiments as further defined by the appended claims.

All of the above-mentioned references are herein incorporated by reference in their entirety to the same extent as if each individual reference was specifically and individually indicated to be incorporated herein by reference in its entirety.

1. A method for treating an inflammatory disease, comprising administering to a subject in need thereof a therapeutically effective amount of a synthetically modified fullerene of the formula
Zₘ—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z and Y are positioned near respective opposite poles of Cₚ;
m is an integer of from 1 to 5 and Z is a hydrophilic, lipophilic, or amphiphilic moiety;
n is an integer of from 1 to 5 and Y is a hydrophilic moiety;
p is an even number between 60 and 200; and
X, if present, represents one or more metal atoms within the fullerene (F), optionally in the form of a trinitride of formula Gᵢ₌₁₋₃Hₖ₌₃₋ᵢN in which G and H are metal atoms.

2. The method of claim 1, wherein p is an even number between 60 and 96.

3. The method of claim 2, wherein p is 60 or 70.

4. The method of claim 1, wherein said synthetically modified fullerene is a prolate ellipsoid shaped fullerene having a major axis such that said poles are located at opposing ends of the major axis of the prolate ellipsoid fullerene.

5. The method of claim 1, wherein said synthetically modified fullerene is spheroid with opposing poles defined by an axis through opposing carbon rings.

6. A method for treating an inflammatory disease, comprising administering to a subject in need thereof a therapeutically effective amount of a synthetically modified fullerene of the formula
Z(Cₚ)Y
wherein p is an even number between 60 and 200; Y is a hydrophilic moiety covalently connected to Cₚ, optionally through a linking group, at or near a pole thereof; and Z is a hydrophilic, lipophilic, or amphiphilic moiety covalently connected to Cₚ, optionally through a linking group, at or near a pole opposite to said Y.

7. The method of claim 6, wherein Cₚ is C₇₀.

8. The method of claim 6, wherein Z comprises at least one —(CH₂)_{q}CH₃ or —(OCH₂CH₂)_{w}OCH₃ moiety, wherein q is an integer of from 5 to 17 and w is an integer of from 1 to 6.

9. The method of claim 6, wherein: (a) Z comprises at least one niacin moiety at a free end thereof; (b) Z comprises at least one —C(O)O— moiety; (c) Y comprises at least one niacin moiety at a free end thereof; or (d) Y comprises at least one —C(O)O— moiety.

10. The method of claim 6, wherein: (a) Z comprises two niacin moieties at two free ends thereof; (b) Z comprises two —C(O)O— moieties; (c) Y comprises two niacin moieties at two free ends thereof; or (d) Y comprises at least two —C(O)O— moieties.

11. The method of claim 6, wherein the synthetically modified fullerene is selected from the group consisting of

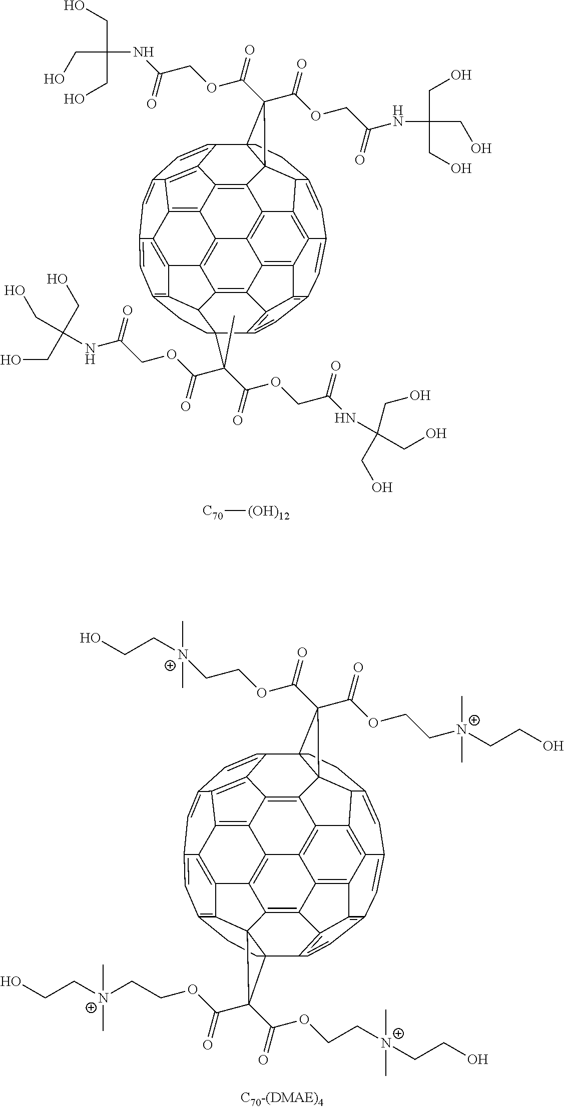

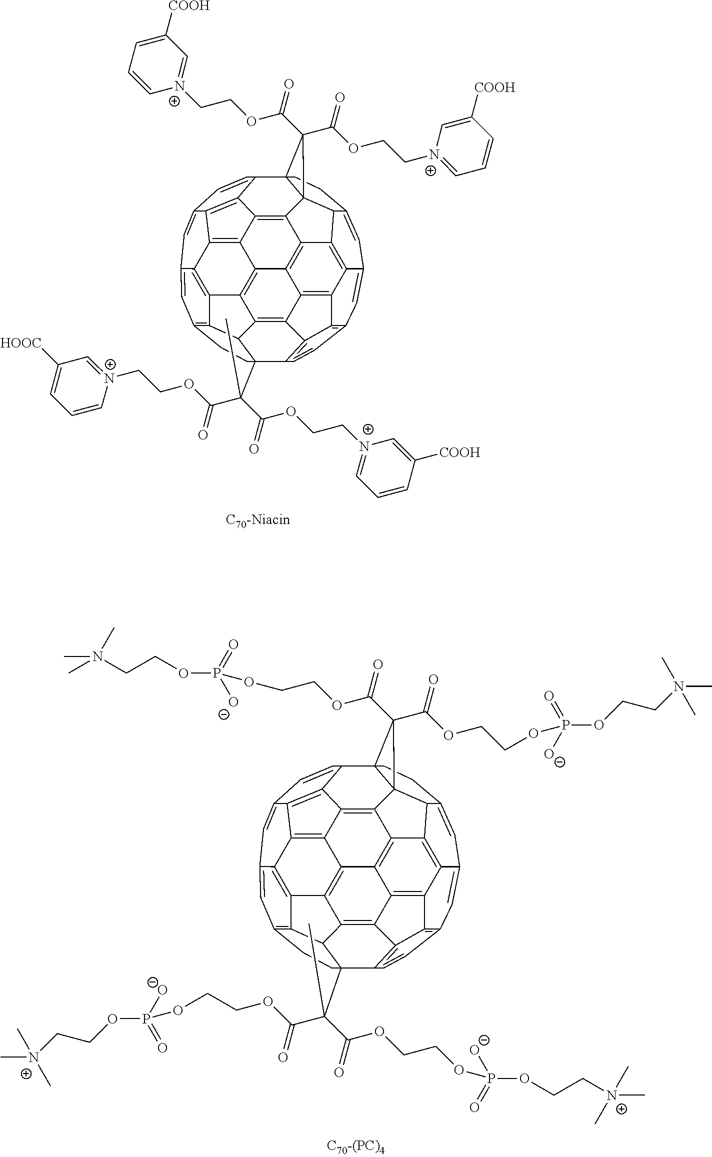

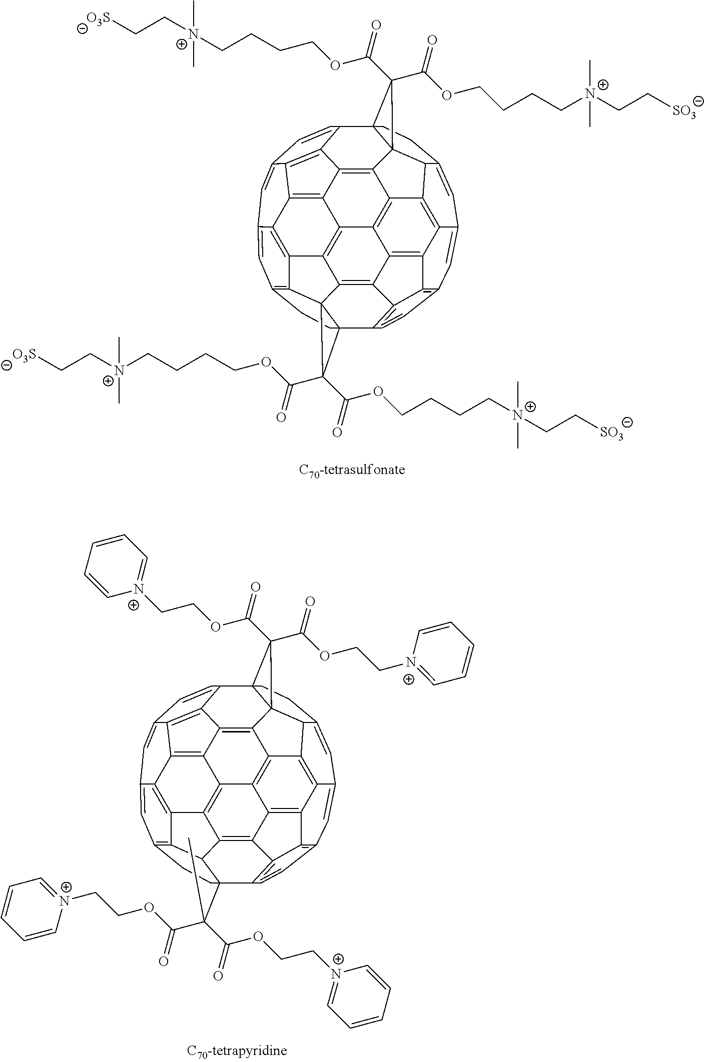

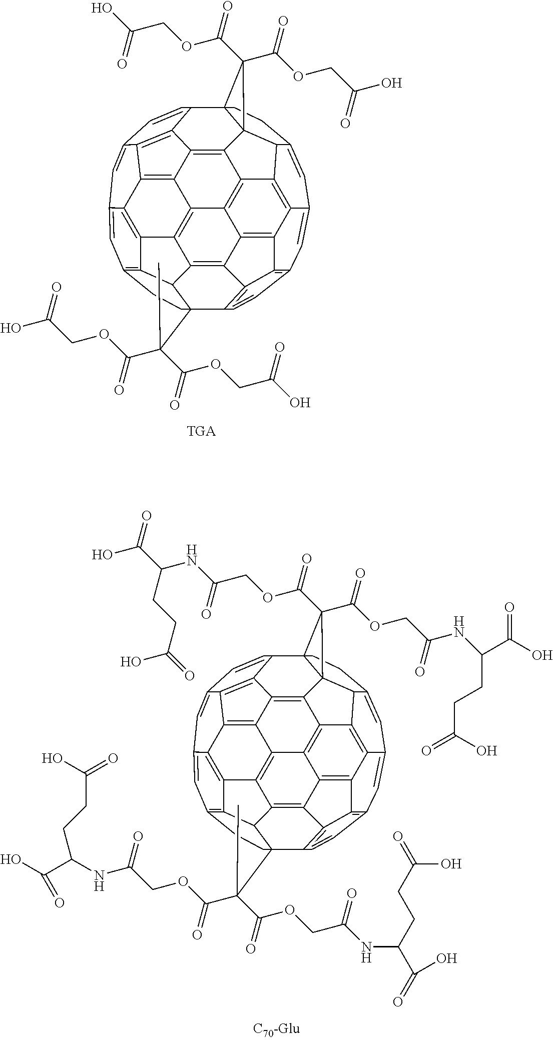

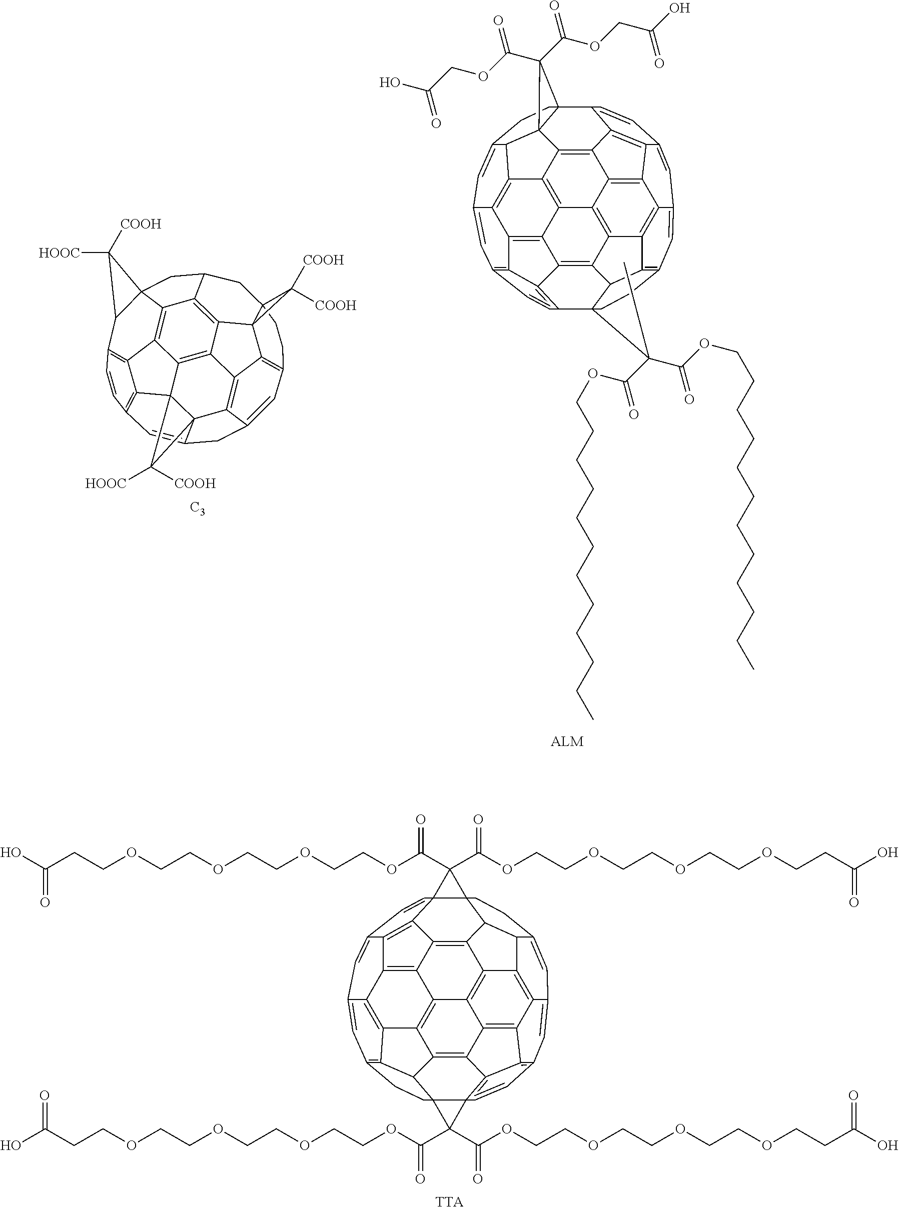

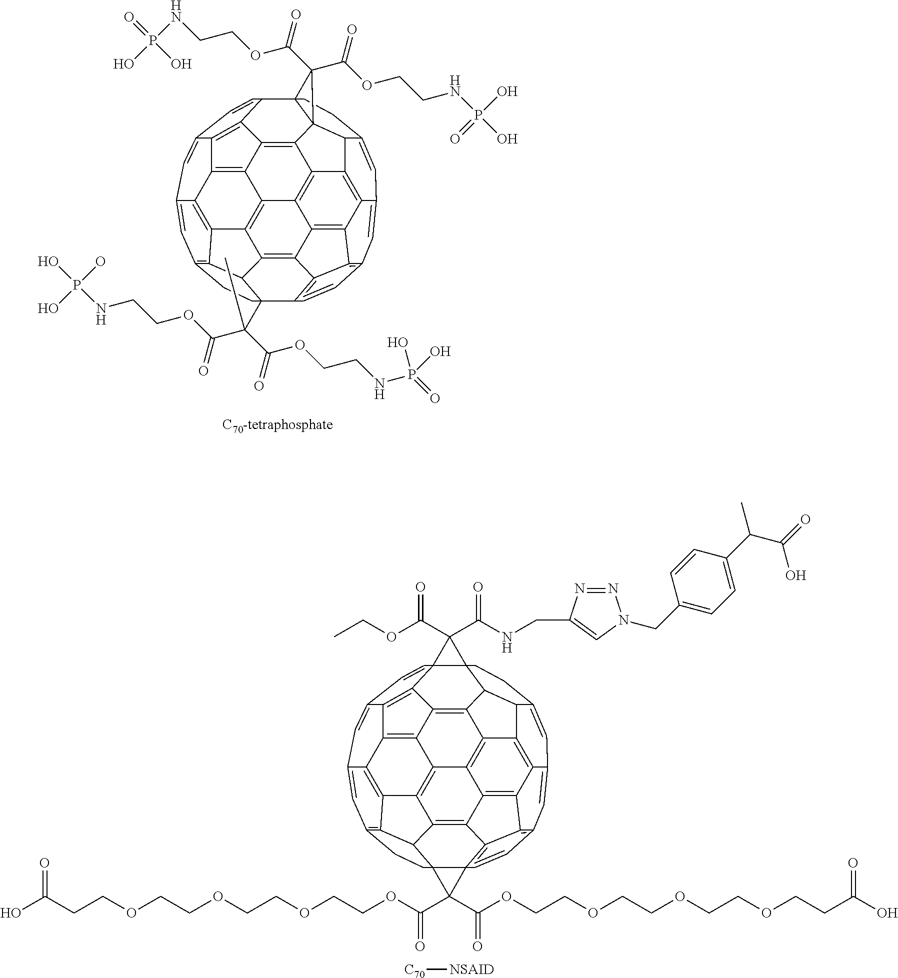

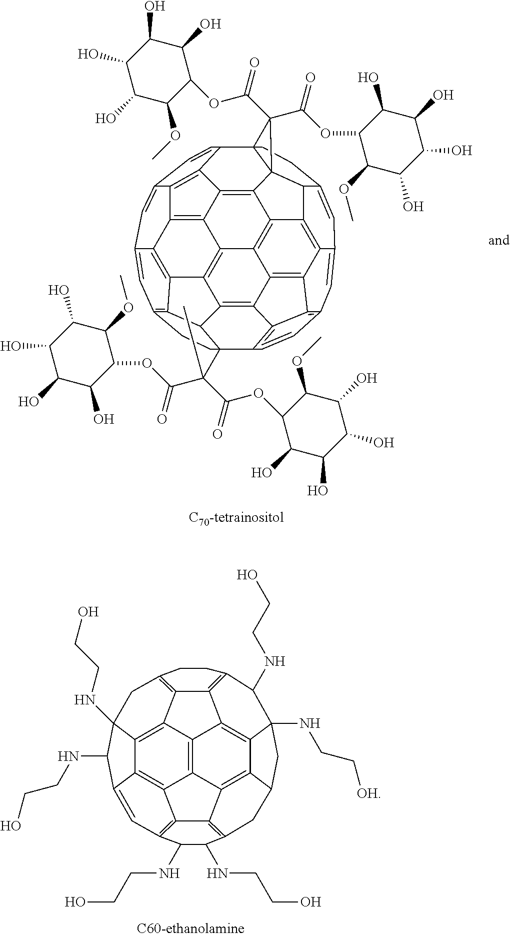

12. The method of claim 6, wherein the inflammatory disease is inflammatory arthritis or an allergic disease.

13. A method of inhibiting build-up of arterial plaque, comprising administering a therapeutically effective amount of one or more fullerenes to a subject in need thereof.

14. The method of claim 13, wherein said fullerenes inhibit accumulation of LDL in foam cells of the subject.

15. The method of claim 13, wherein said fullerenes are delivered directly to the foam cells of the subject.

16. The method of claim 13, wherein said subject is a human.

17. The method of claim 13, wherein at least one of said one or more fullerenes is a synthetically modified fullerene of the formula
Zₘ—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z and Y are positioned near respective opposite poles of Cₚ;
m is an integer of from 1 to 5 and Z is a hydrophilic, lipophilic, or amphiphilic moiety;
n is an integer of from 1 to 5 and Y is a hydrophilic moiety;
p is an even number between 60 and 200; and
X, if present, represents one or more metal atoms within the fullerene (F), optionally in the form of a trinitride of formula Gᵢ₌₁₋₃Hₖ₌₃₋ᵢN in which G and H are metal atoms.

18. The method of claim 17, wherein p is 60 or 70.

19. The method of claim 17, wherein at least one of said one or more fullerenes is a synthetically modified fullerene of the formula
Z(Cₚ)Y
wherein p is an even number between 60 and 200; Y is a hydrophilic moiety covalently connected to Cₚ, optionally through a linking group, at or near a pole thereof, and wherein Z is a hydrophilic, lipophilic, or amphiphilic moiety covalently connected to Cₚ, optionally through a linking group, at or near a pole opposite to said Y.

20. The method of claim 19, wherein Cₚ is C₇₀.

21. The method of claim 19, wherein Z comprises at least one —(CH₂)_{q}CH₃ or —(OCH₂CH₂)_{w}OCH₃ moiety, wherein q is an integer of from 5 to 17 and w is an integer of from 1 to 6.

22. The method of claim 19, wherein: (a) Z comprises at least one niacin moiety at a free end thereof; (b) Z comprises at least one —C(O)O— moiety; (c) Y comprises at least one niacin moiety at a free end thereof; or (d) Y comprises at least one —C(O)O— moiety.

23. The method of claim 19, wherein: (a) Z comprises two niacin moieties at two free ends thereof; (b) Z comprises two —C(O)O— moieties; (c) Y comprises two niacin moieties at two free ends thereof; or (d) Y comprises at least two —C(O)O— moieties.

24. The method of claim 19, wherein the synthetically modified fullerene is selected from the group consisting of

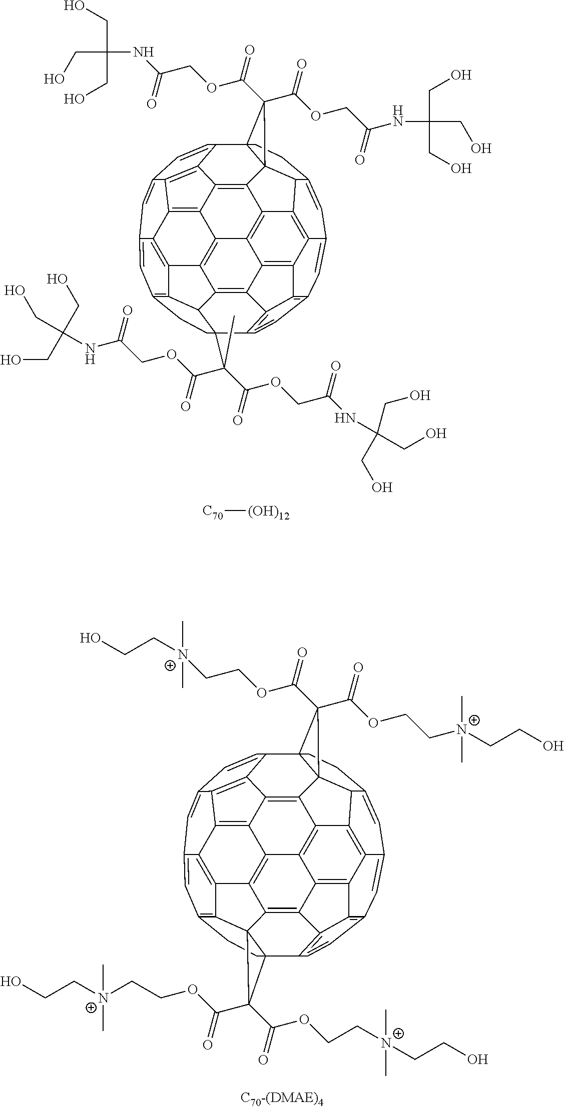

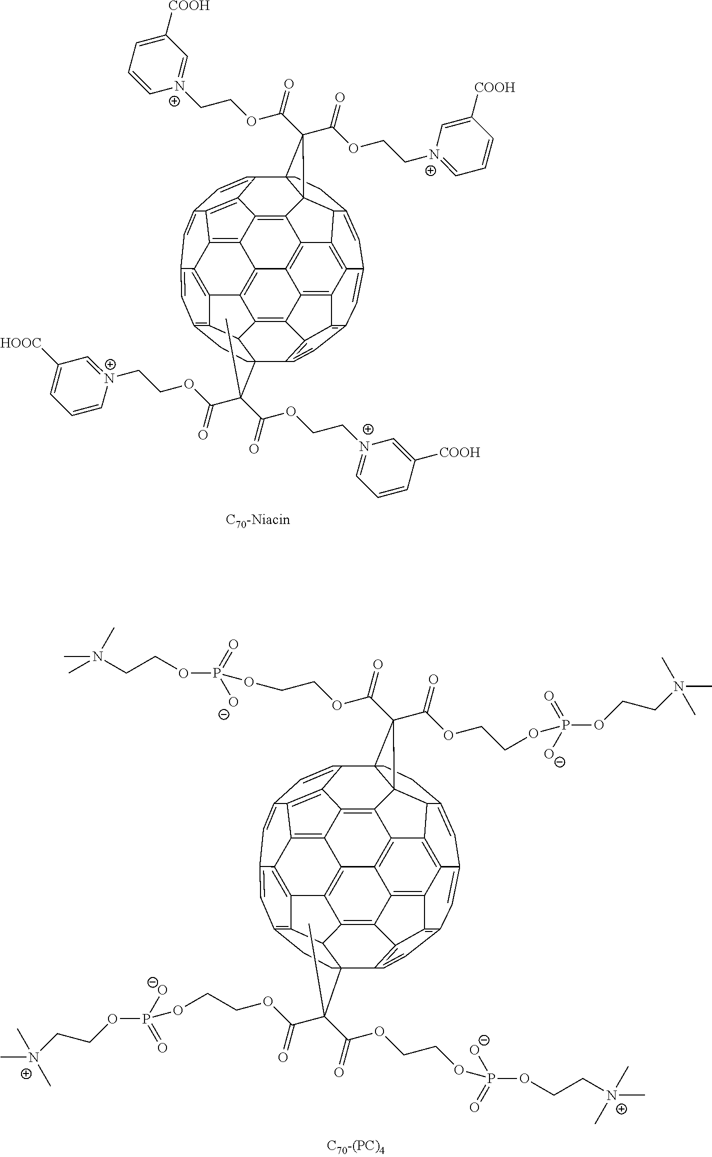

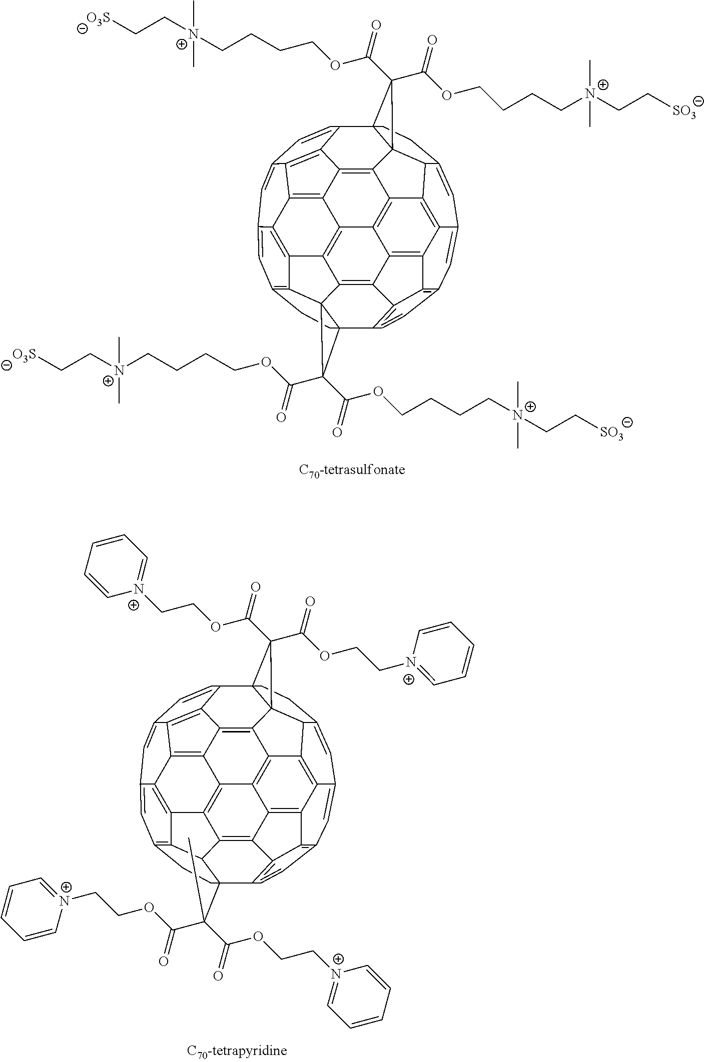

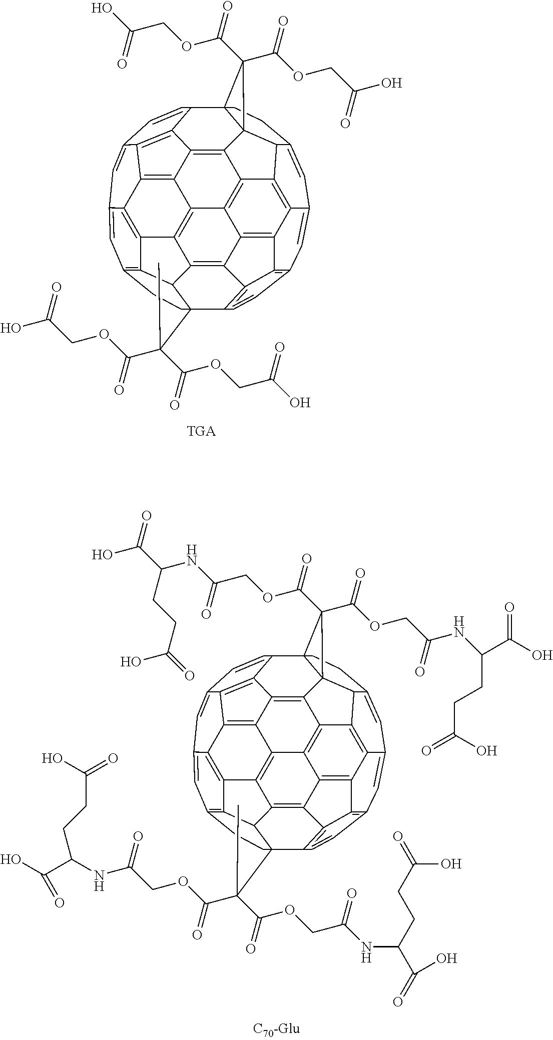

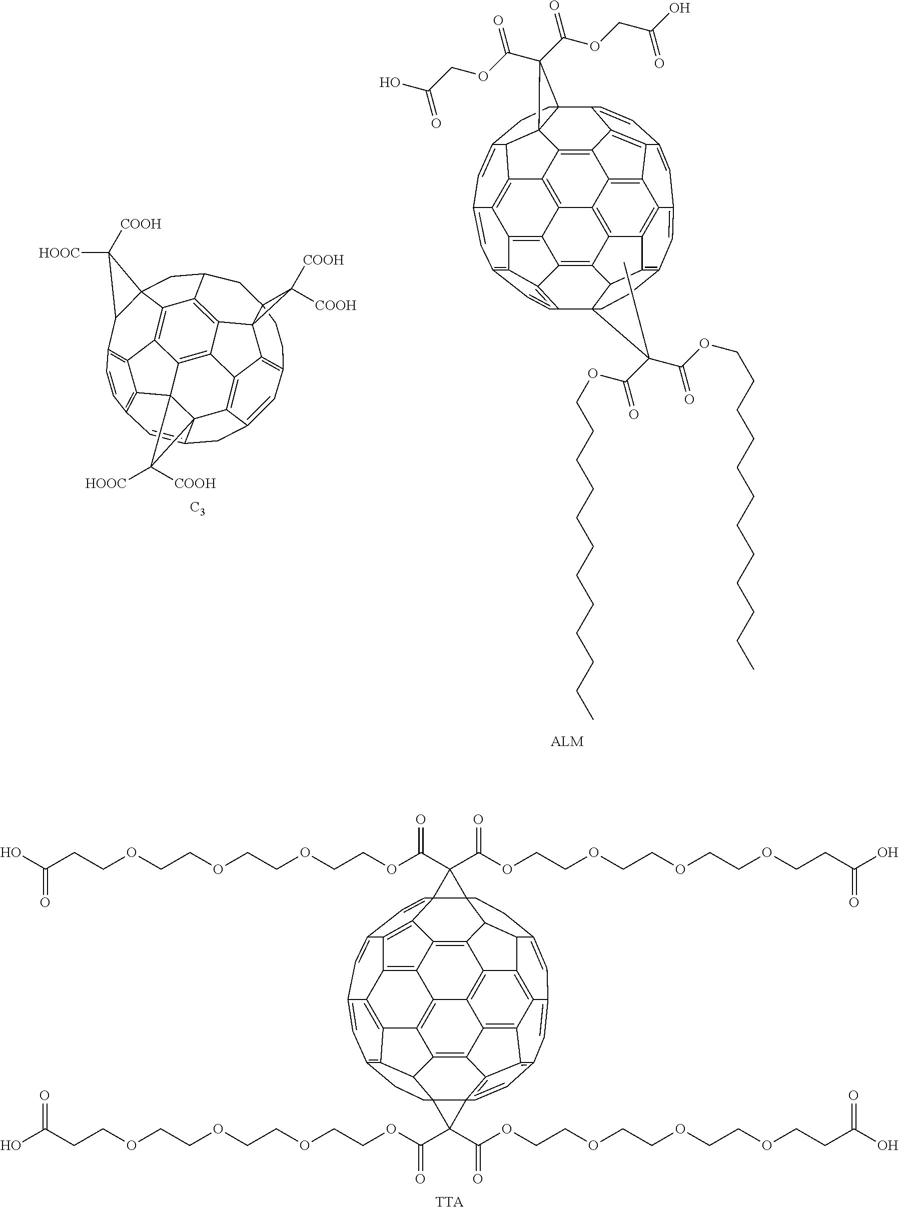

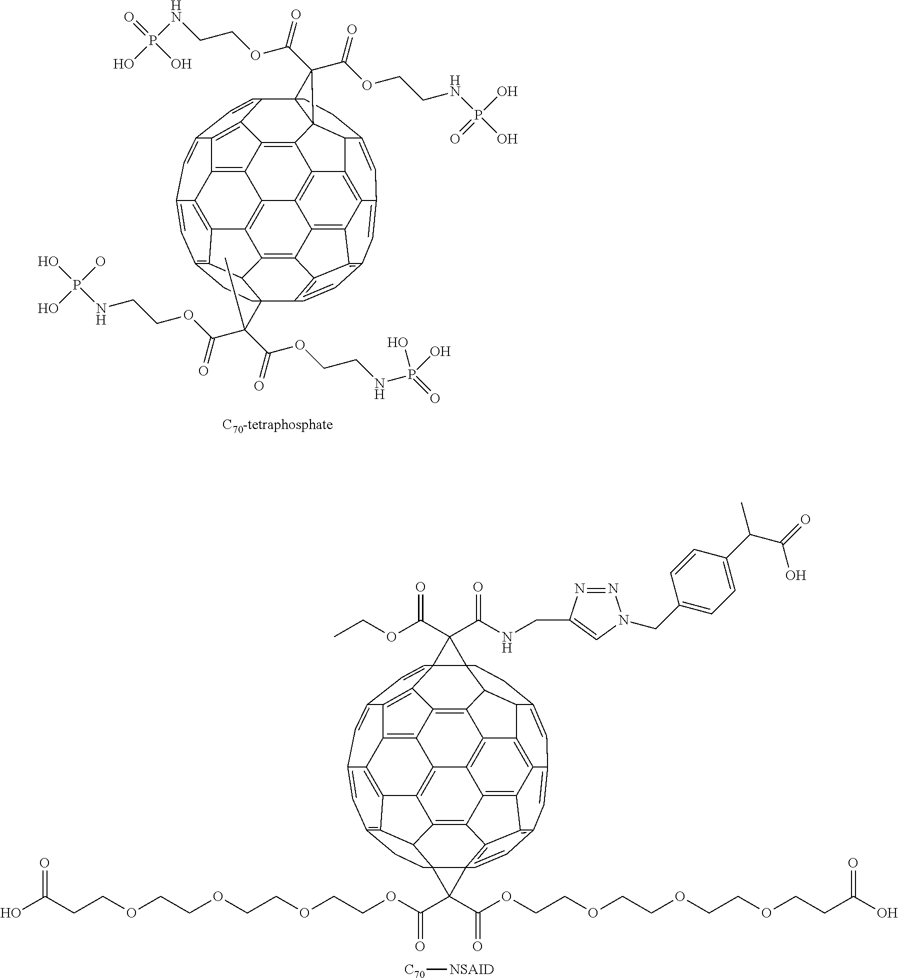

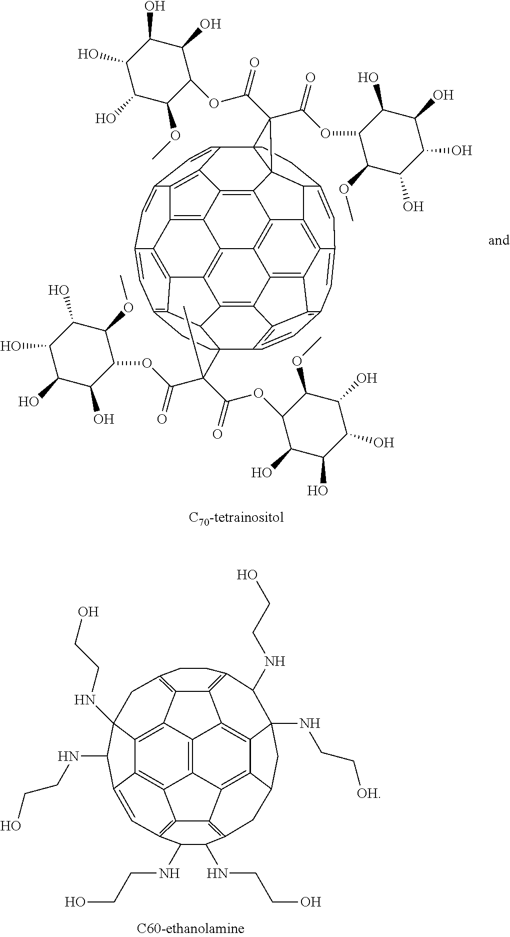

25. A synthetically modified fullerene of the formula
Zₘ—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m is an integer of from 1 to 5 and Z is a hydrophilic, lipophilic, or amphiphilic moiety;
n is an integer of from 1 to 5 and Y is a hydrophilic moiety;
p is an even number between 60 and 200; and
X, if present, represents one or more metal atoms within the fullerene (F), optionally in the form of a trinitride of formula Gᵢ₌₁₋₃Hₖ₌₃₋ᵢN in which G and H are metal atoms,
wherein: (a) Z comprises at least one niacin moiety at a free end thereof; (b) Z comprises at least one —C(O)O— moiety; (c) Y comprises at least one niacin moiety at a free end thereof; or (d) Y comprises at least one —C(O)O— moiety.

26. The method of claim 25, wherein: (a) Z comprises two niacin moieties at two free ends thereof; (b) Z comprises two —C(O)O— moieties; (c) Y comprises two niacin moieties at two free ends thereof; or (d) Y comprises at least two —C(O)O— moieties.

27. A compound selected from the group consisting of is selected from the group consisting of 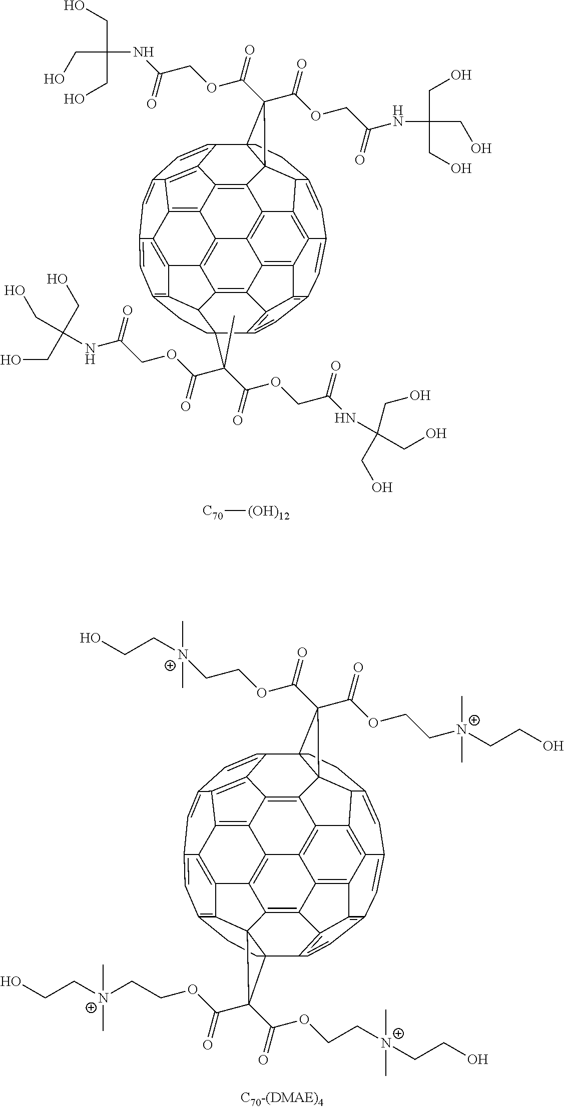 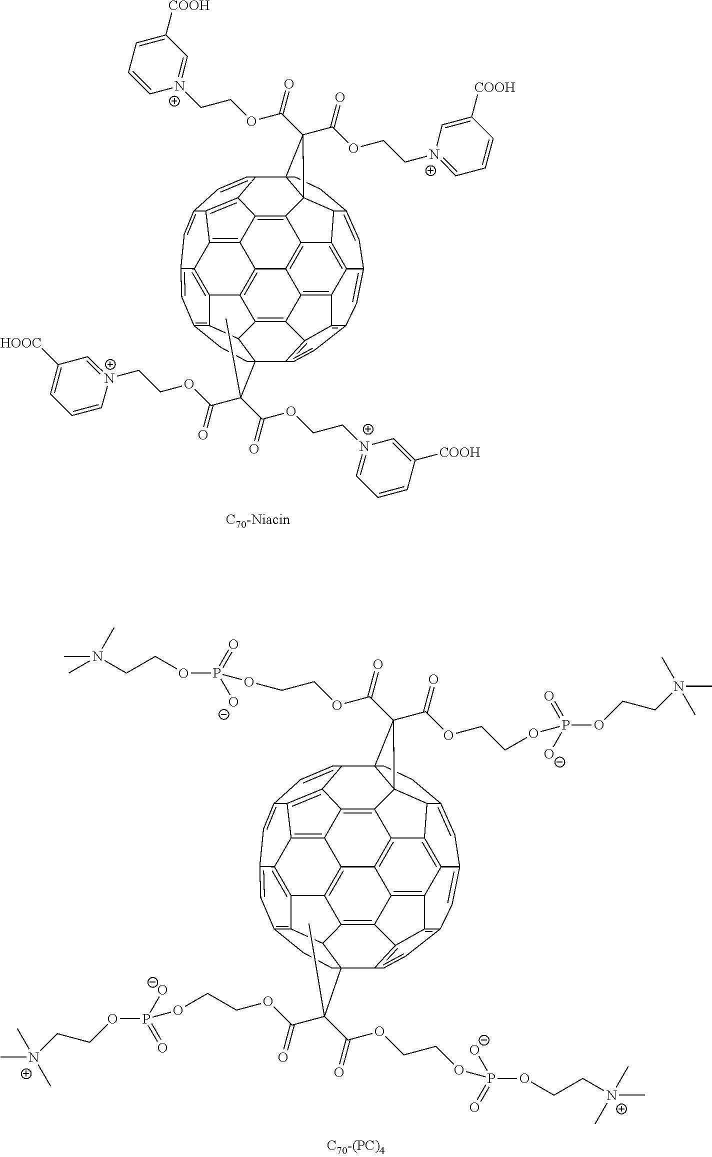 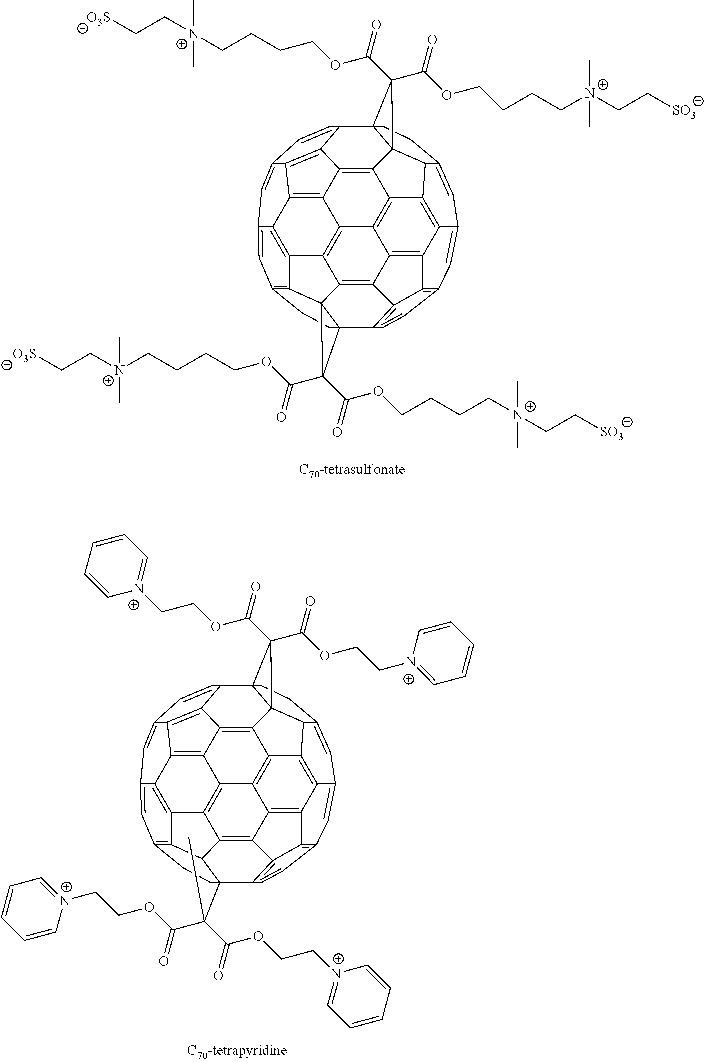 [chemical structure]
